(19) United States
(12) Patent Application Publication
(10) Pub. No.: US 2024/0277858 A1
(43) Pub. Date: Aug. 22, 2024

(54) CD71 BINDING FIBRONECTIN TYPE III DOMAINS

(71) Applicant: Aro Biotherapeutics Company, Philadelphia, PA (US)

(72) Inventors: Russell C. Addis, Philadelphia, PA (US); Zhanna Druzina, Philadelphia, PA (US); Robert Kolakowski, Philadelphia, PA (US); Swapnil Kulkarni, Philadelphia, PA (US); Steven G. Nadler, Philadelphia, PA (US); Karyn O'Neil, Philadelphia, PA (US); Yao Xin, Philadelphia, PA (US)

(21) Appl. No.: 18/174,751

(22) Filed: Feb. 27, 2023

Related U.S. Application Data
(62) Division of application No. 17/070,020, filed on Oct. 14, 2020, now Pat. No. 11,628,222.
(60) Provisional application No. 62/949,020, filed on Dec. 17, 2019.
(60) Provisional application No. 62/914,643, filed on Oct. 14, 2019.

(51) Int. Cl. A61K 47/64; A61K 31/7088; C07K 14/435; C12N 15/113

(52) U.S. Cl. CPC A61K 47/6435; A61K 31/7088; C07K 14/435; C12N 15/113; C12N 2310/11; C12N 2310/14; C12N 2310/3513; C12N 2320/32

(57) ABSTRACT

The present disclosure relates to polypeptides, such as fibronectin type III (FN3) domains that can bind CD71, their conjugates, isolated nucleotides encoding the molecules, vectors, host-cells, as well as methods of making and using the same.

### CROSS-REFERENCE TO RELATED APPLICATIONS

The present application is a continuation of U.S. Non-Provisional application Ser. No. 17/070,020, filed Oct. 14, 2020, which claims priority to U.S. Provisional Application No. 62/914,643, filed Oct. 14, 2019, and U.S. Provisional Application No. 62/949,020, Dec. 17, 2019, each of which is hereby incorporated by reference in its entirety.

### FIELD

The present embodiments relate to fibronectin type III domains (FN3) that specifically bind cluster of differentiation 71 (CD71) and methods of making and using the molecules.

### BACKGROUND

CD71, also known as transferrin receptor 1, is transmembrane that is essential for iron transport into cells. It is highly expressed on many tumor types and at the blood brain barrier, and has thus become an important target for drug delivery. Following binding to iron loaded transferrin, CD71 is rapidly endocytosed and efficiently recycled back to the cell surface. Studies with CD71 antibody drug conjugates suggest that targeting CD71 can improve specificity and selectivity of drug delivery and widen the therapeutic index. In addition, studies using anti-CD71 monoclonal antibodies indicate that binding affinity can play an important role in enabling blood brain barrier transcytosis. Antibodies with high affinity for CD71 are rapidly internalized and alter normal receptor trafficking so that instead of recycling, the receptor is targeted to the lysosome for degradation. In contrast, antibodies with low affinity for CD71 allow for receptor recycling and higher brain exposure.

While antibodies or antibody fragments are the most widely used class of therapeutic proteins when high affinity and specificity for a target molecule are desired, non-antibody proteins can be engineered to also bind such targets. These “alternative scaffold” proteins have advantages over traditional antibodies due to their small size, lack of disulphide bonds, high stability, ability to be expressed in prokaryotic hosts, easy purification, and they are easily conjugated to drugs/toxins, penetrate efficiently into tissues and are readily formatted into multispecific binders.

One such alternative scaffold is the immunoglobulin (Ig) fold. This fold is found in the variable regions of antibodies, as well as thousands of non-antibody proteins. It has been shown that one such Ig protein, the tenth fibronectin type III (FN3) repeat from human fibronectin, can tolerate a number of mutations in surface exposed loops while retaining the overall Ig-fold structure. Thus, what is needed is a FN3 domain that can specifically bind to CD71, and methods of using such molecules for cancer therapy.

### SUMMARY

In some embodiments, FN3 domains (e.g. polypeptides) that specifically bind CD71 protein are provided. In some embodiments, the FN3 domains are isolated. In some embodiments, the FN3 domains are recombinant. In some embodiments, the FN3 domains are non-naturally occurring.

In some embodiments, FN3 domains are provided that comprise the amino acid sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61 or 62. In some embodiments, FN3 domains are provided that comprise the amino acid sequence of SEQ ID NOs: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, or 149. In some embodiments, FN3 domains are provided that comprise the amino acid sequence of SEQ ID NOs 81-309: In some embodiments, the FN3 domains bind to CD71. In some embodiments, the FN3 domain binds to human CD71 at a site on CD71 that does not compete with transferrin binding to CD71. In some embodiments, FN3 domains are provided that comprise the amino acid sequence of SEQ ID NOs: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, or 149. In some embodiments, the FN3 domains specifically bind to CD71. In some embodiments, the polypeptide is provided that comprises more than one FN3 domains connected by a linker, such as a flexible linker. In some embodiments, the polypeptide comprises 2, 3, or 4 FN3 domains that are connected to one another by one or more linkers between the domains.

In some embodiments, isolated polynucleotides encoding the FN3 domains described herein are provided.

In some embodiments, a vector comprising the polynucleotides described herein are provided.

In some embodiments, a host cell comprising the vectors described herein are provided.

In some embodiments, methods of producing the FN3 domains are provided. In some embodiments, the method comprises culturing a host cell comprising a vector encoding or expressing the FN3 domain. In some embodiments, the method further comprises purifying the FN3 domain. In some embodiments, the FN3 domain specifically binds CD71.

In some embodiments, pharmaceutical compositions comprising a FN3 domain that binds to CD71 and a pharmaceutically acceptable carrier are provided.

In some embodiments, anti-idiotypic antibodies that binds a FN3 domain that binds to CD71 are provided.

In some embodiments, kits comprising one or more of the FN3 domains are provided.

In some embodiments, methods of detecting CD71-expressing cancer cells in a tumor tissue are provided. In some embodiments, the method comprises obtaining a sample of the tumor tissue from a subject and detecting whether CD71 protein is expressed in the tumor tissue by contacting the sample of the tumor tissue with the FN3 domain that binds CD71 protein comprising the amino acid sequence of one of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309 and detecting the binding between CD71 protein and the FN3 domain.

In some embodiments, methods of isolating CD71 expressing cells are provided. In some embodiments, the method comprises obtaining a sample from a subject; contacting the sample with the FN3 domain that binds CD71 protein comprising the amino acid sequence of one of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309 and isolating the cells bound to the FN3 domains.

In some embodiments, methods of detecting CD71-expressing cancer cells in a tumor tissue are provided. In some embodiments, the method comprises conjugating the FN3 domain that binds CD71 protein comprising the amino acid sequence of one of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309 to a detectable label to form a conjugate; administering the conjugate to a subject; and visualizing the CD71 expressing cancer cells to which the conjugate is bound.

In some embodiments, methods of treating cancer in a subject in need thereof are provided. In some embodiments, the method comprises administering a polypeptide that binds to CD71. In some embodiments, that the polypeptide is a FN3 domain that binds to CD71. In some embodiments, the polypeptide comprises a sequence such as SEQ ID Nos: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, 149, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309, or a polypeptide as provided herein that is linked to or conjugated to a therapeutic agent.

In some embodiments, methods of treating a neurological condition and/or a brain tumor are provided. In some embodiments, the methods comprise administering to the subject a polypeptide or the pharmaceutical composition as provided herein. In some embodiments, the brain tumor is selected from the group consisting of nonmalignant, benign, and malignant brain tumors.

In some embodiments, methods of delivering an agent of interest to a CD71 positive cell are provided. In some embodiments, the methods comprise contacting a cell with the agent of interest coupled to a FN3 domain that binds to CD71, such as a polypeptide as provided herein. In some embodiments, the agent of interest is a chemotherapeutic agent, a drug, a growth inhibitory agent, a toxin, a radioactive isotope, an anti-tubulin agent, a polynucleotide, a siRNA molecule, an antisense molecule, a RNA molecule, a DNA molecule, DNA minor groove binders, DNA replication inhibitors, alkylating agents, antibiotics, antifolates, antimetabolites, chemotherapy sensitizers, topoisomerase inhibitors, or a vinca alkaloid.

In some embodiments, the polypeptide is a FN3 protein that binds to CD71 at a site that does not compete or inhibit transferrin binding to CD71.

In some embodiments, methods of identifying a FN3 protein that binds to CD71 at a site that does not compete or inhibit transferrin binding to CD71 are provided.

### DETAILED DESCRIPTION OF THE DISCLOSURE

As used in this specification and the appended claims, the singular forms “a,” “an,” and “the” include plural referents unless the content clearly dictates otherwise. Thus, for example, reference to “a cell” includes a combination of two or more cells, and the like.

“Fibronectin type III (FN3) domain” (FN3 domain) refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3^{rd} FN3 domain of tenascin (TN3), or the 10^{th} FN3 domain of fibronectin (FN10).

The term “capture agent” refers to substances that bind to a particular type of cells and enable the isolation of that cell from other cells. Exemplary capture agents are magnetic beads, ferrofluids, encapsulating reagents, molecules that bind the particular cell type and the like.

“Sample” refers to a collection of similar fluids, cells, or tissues isolated from a subject, as well as fluids, cells, or tissues present within a subject. Exemplary samples are tissue biopsies, fine needle aspirations, surgically resected tissue, organ cultures, cell cultures and biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium and lavage fluids and the like.

“Substituting” or “substituted” or “mutating” or “mutated” refers to altering, deleting of inserting one or more amino acids or nucleotides in a polypeptide or polynucleotide sequence to generate a variant of that sequence.

“Variant” refers to a polypeptide or a polynucleotide that differs from a reference polypeptide or a reference polynucleotide by one or more modifications for example, substitutions, insertions or deletions.

“Specifically binds” or “specific binding” refers to the ability of a FN3 domain to bind to its target, such as CD71, with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Alternatively, “specific binding” refers to the ability of a FN3 domain to bind to its target (e.g. CD71) at least 5-fold above a negative control in standard ELISA assay. In some embodiments, a negative control is an FN3 domain that does not bind CD71. In some embodiment, an FN3 domain that specifically binds CD71 may have cross-reactivity to other related antigens, for example to the same predetermined antigen from other species (homologs), such as Macaca Fascicularis (cynomolgous monkey, cyno) or Pan troglodytes (chimpanzee).

“Library” refers to a collection of variants. The library may be composed of polypeptide or polynucleotide variants.

“Stability” refers to the ability of a molecule to maintain a folded state under physiological conditions such that it retains at least one of its normal functional activities, for example, binding to a predetermined antigen such as CD71.

“CD71” refers to human CD71 protein having the amino acid sequence of SEQ ID NOs: 32 or 80. In some embodiments, SEQ ID NO: 32 is full length human CD71 protein. In some embodiments, SEQ ID NO: 80 is the extracellular domain of human CD71.

“Tencon” refers to the synthetic fibronectin type III (FN3) domain having the sequence shown in SEQ ID NO: 1 and described in U.S. Pat. Publ. No. 2010/0216708.

A “cancer cell” or a “tumor cell” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo (Freshney, Culture of Animal Cells: A Manual of Basic Technique (3rd ed. 1994)).

“Vector” refers to a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Expression vector” refers to a vector that can be utilized in a biological system or in a reconstituted biological system to direct the translation of a polypeptide encoded by a polynucleotide sequence present in the expression vector.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than about 50 amino acids may be referred to as “peptides”.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Subject” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. Except when noted, the terms “patient” or “subject” are used interchangeably.

“Isolated” refers to a homogenous population of molecules (such as synthetic polynucleotides or a polypeptide such as FN3 domains) which have been substantially separated and/or purified away from other components of the system the molecules are produced in, such as a recombinant cell, as well as a protein that has been subjected to at least one purification or isolation step. “Isolated FN3 domain” refers to an FN3 domain that is substantially free of other cellular material and/or chemicals and encompasses FN3 domains that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

### Compositions of Matter

In some embodiments, proteins comprising a polypeptide comprising an amino acid sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309 are provided. In some embodiments, proteins comprising a polypeptide comprising an amino acid sequence that is at least 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to a sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309. In some embodiments, the protein is at least 85%, 86%, 87%, 88%, 89%, 90%, 91%, 92%, 93%, 94%, 95%, 96%, 97%, 98% or 99% identical to a sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309. In some embodiments, the protein is at least 90%, 91%, 92%, 93%, 94%, 95%, 96%, 97%, 98% or 99% identical to a sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309. In some embodiments, the protein is at least 95%, 96%, 97%, 98% or 99% identical to a sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309.

The polypeptides provided herein can be part of a larger polypeptide and can be referred to as a domain. The homology or identity between two domains in different polypeptides is based on the domains that are similar as opposed to the overall polypeptide. For example, if a polypeptide comprises a polypeptide comprising a FN3 domain comprising SEQ ID NO: 81 and said domain is conjugated to a scFV antibody, another protein that has a domain that is similar but not identical to SEQ ID NO: 81 can be at least 90% identical even if the scFV shares no homology. Thus, the % identity can be based on the domain or on the entire length of the polypeptide. Methods of determining % identity are provided for herein or are known to one of skill in the art.

In some embodiments, fibronectin type III (FN3) domains that bind or specifically bind human CD71 protein (SEQ ID Nos: 32 or 80) are provided. As provided herein, the FN3 domains can bind to the CD71 protein. Also provided, even if not explicitly stated is that the domains can also specifically bind to the CD71 protein. Thus, for example, a FN3 domain that binds to CD71 would also encompass a FN3 domain protein that specifically binds to CD71. These molecules can be used, for example, in therapeutic and diagnostic applications and in imaging. In some embodiments, polynucleotides encoding the FN3 domains disclosed herein or complementary nucleic acids thereof, vectors, host cells, and methods of making and using them are provided.

In some embodiments, an isolated FN3 domain that binds or specifically binds CD71 is provided.

In some embodiments, the FN3 domain comprises two FN3 domains connected by a linker. The linker can be a flexible linker. The linker can be a short peptide sequence, such as those described herein. For example, the linker can be a G/S linker and the like.

In some embodiments, the FN3 domain may bind CD71 with a dissociation constant (K_{D}) of less than about 1×10⁻⁷ M, for example less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are made with standardized solutions of protein scaffold and antigen, and a standardized buffer, such as the buffers described herein.

In some embodiments, the FN3 domain may bind CD71 at least 5-fold above the signal obtained for a negative control in a standard ELISA assay.

In some embodiments, the FN3 domain that binds or specifically binds CD71 comprises an initiator methionine (Met) linked to the N-terminus of the molecule. In some embodiments, the FN3 domain that binds or specifically binds CD71 comprises a cysteine (Cys) linked to a C-terminus of the FN3 domain. The addition of the N-terminal Met and/or the C-terminal Cys may facilitate expression and/or conjugation of half-life extending molecules.

The FN3 domain can also contain cysteine substitutions, such as those that are described in U.S. Pat. No. 10,196,446, which is hereby incorporated by reference in its entirety. Briefly, in some embodiments, the polypeptides provided herein can comprise at least one cysteine substitution at a position selected from the group consisting of residues 6, 8, 10, 11, 14, 15, 16, 20, 30, 34, 38, 40, 41, 45, 47, 48, 53, 54, 59, 60, 62, 64, 70, 88, 89, 90, 91, and 93 of the FN3 domain based on SEQ ID NO: 6 or SEQ ID NO: 1 of U.S. Pat. No. 10,196,446, and the equivalent positions in related FN3 domains. In some embodiments, the substitution is at residue 6. In some embodiments, the substitution is at residue 8. In some embodiments, the substitution is at residue 10. In some embodiments, the substitution is at residue 11. In some embodiments, the substitution is at residue 14. In some embodiments, the substitution is at residue 15. In some embodiments, the substitution is at residue 16. In some embodiments, the substitution is at residue 20. In some embodiments, the substitution is at residue 30. In some embodiments, the substitution is at residue 34. In some embodiments, the substitution is at residue 38. In some embodiments, the substitution is at residue 40. In some embodiments, the substitution is at residue 41. In some embodiments, the substitution is at residue 45. In some embodiments, the substitution is at residue 47. In some embodiments, the substitution is at residue 48. In some embodiments, the substitution is at residue 53. In some embodiments, the substitution is at residue 54. In some embodiments, the substitution is at residue 59. In some embodiments, the substitution is at residue 60. In some embodiments, the substitution is at residue 62. In some embodiments, the substitution is at residue 64. In some embodiments, the substitution is at residue 70. In some embodiments, the substitution is at residue 88. In some embodiments, the substitution is at residue 89. In some embodiments, the substitution is at residue 90. In some embodiments, the substitution is at residue 91. In some embodiments, the substitution is at residue 93.

A cysteine substitution at a position in the domain or protein comprises a replacement of the existing amino acid residue with a cysteine residue. Other examples of cysteine modifications can be found in, for example, U.S. Patent Application Publication No. 20170362301, which is hereby incorporated by reference in its entirety. The alignment of the sequences can be performed using BlastP using the default parameters at, for example, the NCBI website.

In some embodiments, the FN3 domain that binds CD71 is internalized into a cell. In some embodiments, internalization of the FN3 domain may facilitate delivery of a detectable label or therapeutic into a cell. In some embodiments, internalization of the FN3 domain may facilitate delivery of a cytotoxic agent into a cell. The cytotoxic agent can act as a therapeutic agent. In some embodiments, internalization of the FN3 domain may facilitate the delivery of any detectable label, therapeutic, and/or cytotoxic agent disclosed herein into a cell. In some embodiments, the cell is a tumor cell. In some embodiments, the cell is a liver cell.

In some embodiments, the FN3 domain that binds CD71 is based on Tencon sequence of SEQ ID NO: 1 or Tencon 27 sequence of SEQ ID NO: 4, optionally having substitutions at residues positions 11, 14, 17, 37, 46, 73, or 86 (residue numbering corresponding to SEQ ID NO: 4).

In some embodiments, an isolated FN3 domain that binds CD71 comprises the amino acid sequence of SEQ ID NOs: 33, 34, 35, 36, 37, 38, 39, 40, 41, 42, 43, 44, 45, 46, 47, 48, 49, or 50.

In some embodiments, an isolated FN3 domain that binds CD71 comprises the amino acid sequence of SEQ ID NOs: 51, 52, 53, 54, 55, 56, 57, 58, 59, 60, or 61.

In some embodiments, an isolated FN3 domain that binds CD71 comprises the amino acid sequence of SEQ ID NO: 33.

In some embodiments, an isolated FN3 domain that binds CD71 comprises the amino acid sequence of SEQ ID NO: 34.

In some embodiments, an isolated FN3 domain that binds CD71 comprises the amino acid sequence of SEQ ID NO: 35.

In some embodiments, an isolated FN3 domain that binds CD71 comprises the amino acid sequence of SEQ ID NO: 36.

In some embodiments, an isolated FN3 domain that binds CD71 comprises the amino acid sequence of SEQ ID NO: 37.

In some embodiments, an isolated FN3 domain that binds CD71 comprises the amino acid sequence of SEQ ID NO: 38.

In some embodiments, an isolated FN3 domain that binds CD71 comprises the amino acid sequence of SEQ ID NO: 39.

In some embodiments, an isolated FN3 domain that binds CD71 comprises the amino acid sequence of SEQ ID NO: 40.

In some embodiments, an isolated FN3 domain that binds CD71 comprises the amino acid sequence of SEQ ID NO: 41.

In some embodiments, an isolated FN3 domain that binds CD71 comprises the amino acid sequence of SEQ ID NO: 42.

In some embodiments, an isolated FN3 domain that binds CD71 comprises the amino acid sequence of SEQ ID NO: 43.

In some embodiments, an isolated FN3 domain that binds CD71 comprises the amino acid sequence of SEQ ID NO: 44.

In some embodiments, an isolated FN3 domain that binds CD71 comprises the amino acid sequence of SEQ ID NO: 45.

In some embodiments, an isolated FN3 domain that binds CD71 comprises the amino acid sequence of SEQ ID NO: 46.

In some embodiments, an isolated FN3 domain that binds CD71 comprises the amino acid sequence of SEQ ID NO: 47.

In some embodiments, an isolated FN3 domain that binds CD71 comprises the amino acid sequence of SEQ ID NO: 48.

In some embodiments, an isolated FN3 domain that binds CD71 comprises the amino acid sequence of SEQ ID NO: 49.

In some embodiments, an isolated FN3 domain that binds CD71 comprises the amino acid sequence of SEQ ID NO: 50.

In some embodiments, an isolated FN3 domain that binds CD71 comprises the amino acid sequence of SEQ ID NO: 51.

In some embodiments, an isolated FN3 domain that binds CD71 comprises the amino acid sequence of SEQ ID NO: 52.

In some embodiments, an isolated FN3 domain that binds CD71 comprises the amino acid sequence of SEQ ID NO: 53.

In some embodiments, an isolated FN3 domain that binds CD71 comprises the amino acid sequence of SEQ ID NO: 54.

In some embodiments, an isolated FN3 domain that binds CD71 comprises the amino acid sequence of SEQ ID NO: 55.

In some embodiments, an isolated FN3 domain that binds CD71 comprises the amino acid sequence of SEQ ID NO: 56.

In some embodiments, an isolated FN3 domain that binds CD71 comprises the amino acid sequence of SEQ ID NO: 57.

In some embodiments, an isolated FN3 domain that binds CD71 comprises the amino acid sequence of SEQ ID NO: 58.

In some embodiments, an isolated FN3 domain that binds CD71 comprises the amino acid sequence of SEQ ID NO: 59.

In some embodiments, an isolated FN3 domain that binds CD71 comprises the amino acid sequence of SEQ ID NO: 60.

In some embodiments, an isolated FN3 domain that binds CD71 comprises the amino acid sequence of SEQ ID NO: 61.

In some embodiments, an isolated FN3 domain that binds CD71 comprises the amino acid sequence of SEQ ID NO: 62.

In some embodiments, an isolated FN3 domain that binds CD71 comprises the amino acid sequence of SEQ ID NO: 81. In some embodiments, an isolated FN3 domain that binds CD71 comprises the amino acid sequence of SEQ ID NO: 82. In some embodiments, an isolated FN3 domain that binds CD71 comprises the amino acid sequence of SEQ ID NO: 83. In some embodiments, an isolated FN3 domain that binds CD71 comprises the amino acid sequence of SEQ ID NO: 84. In some embodiments, an isolated FN3 domain that binds CD71 comprises the amino acid sequence of SEQ ID NO: 85. In some embodiments, an isolated FN3 domain that binds CD71 comprises the amino acid sequence of SEQ ID NO: 86. In some embodiments, an isolated FN3 domain that binds CD71 comprises the amino acid sequence of SEQ ID NO: 87. In some embodiments, an isolated FN3 domain that binds CD71 comprises the amino acid sequence of SEQ ID NO: 88. In some embodiments, an isolated FN3 domain that binds CD71 comprises the amino acid sequence of SEQ ID NO: 89. In some embodiments, an isolated FN3 domain that binds CD71 comprises the amino acid sequence of SEQ ID NO: 90. In some embodiments, an isolated FN3 domain that binds CD71 comprises the amino acid sequence of SEQ ID NO: 91. In some embodiments, an isolated FN3 domain that binds CD71 comprises the amino acid sequence of SEQ ID NO: 92. In some embodiments, an isolated FN3 domain that binds CD71 comprises the amino acid sequence of SEQ ID NO: 93. In some embodiments, an isolated FN3 domain that binds CD71 comprises the amino acid sequence of SEQ ID NO: 94. In some embodiments, an isolated FN3 domain that binds CD71 comprises the amino acid sequence of SEQ ID NO: 95. In some embodiments, an isolated FN3 domain that binds CD71 comprises the amino acid sequence of SEQ ID NO: 96. In some embodiments, an isolated FN3 domain that binds CD71 comprises the amino acid sequence of SEQ ID NO: 97. In some embodiments, an isolated FN3 domain that binds CD71 comprises the amino acid sequence of SEQ ID NO: 98. In some embodiments, an isolated FN3 domain that binds CD71 comprises the amino acid sequence of SEQ ID NO: 99. In some embodiments, an isolated FN3 domain that binds CD71 comprises the amino acid sequence of SEQ ID NO: 100. In some embodiments, an isolated FN3 domain that binds CD71 comprises the amino acid sequence of SEQ ID NO: 101. In some embodiments, an isolated FN3 domain that binds CD71 comprises the amino acid sequence of SEQ ID NO: 102. In some embodiments, an isolated FN3 domain that binds CD71 comprises the amino acid sequence of SEQ ID NO: 103. In some embodiments, an isolated FN3 domain that binds CD71 comprises the amino acid sequence of SEQ ID NO: 104. In some embodiments, an isolated FN3 domain that binds CD71 comprises the amino acid sequence of SEQ ID NO: 105. In some embodiments, an isolated FN3 domain that binds CD71 comprises the amino acid sequence of SEQ ID NO: 106. In some embodiments, an isolated FN3 domain that binds CD71 comprises the amino acid sequence of SEQ ID NO: 107. In some embodiments, an isolated FN3 domain that binds CD71 comprises the amino acid sequence of SEQ ID NO: 108. In some embodiments, an isolated FN3 domain that binds CD71 comprises the amino acid sequence of SEQ ID NO: 109. In some embodiments, an isolated FN3 domain that binds CD71 comprises the amino acid sequence of SEQ ID NO: 110. In some embodiments, an isolated FN3 domain that binds CD71 comprises the amino acid sequence of SEQ ID NO: 111. In some embodiments, an isolated FN3 domain that binds CD71 comprises the amino acid sequence of SEQ ID NO: 112. In some embodiments, an isolated FN3 domain that binds CD71 comprises the amino acid sequence of SEQ ID NO: 113. In some embodiments, an isolated FN3 domain that binds CD71 comprises the amino acid sequence of SEQ ID NO: 114. In some embodiments, an isolated FN3 domain that binds CD71 comprises the amino acid sequence of SEQ ID NO: 115. In some embodiments, an isolated FN3 domain that binds CD71 comprises the amino acid sequence of SEQ ID NO: 116. In some embodiments, an isolated FN3 domain that binds CD71 comprises the amino acid sequence of SEQ ID NO: 117. In some embodiments, an isolated FN3 domain that binds CD71 comprises the amino acid sequence of SEQ ID NO: 118. In some embodiments, an isolated FN3 domain that binds CD71 comprises the amino acid sequence of SEQ ID NO: 119. In some embodiments, an isolated FN3 domain that binds CD71 comprises the amino acid sequence of SEQ ID NO: 120. In some embodiments, an isolated FN3 domain that binds CD71 comprises the amino acid sequence of SEQ ID NO: 121. In some embodiments, an isolated FN3 domain that binds CD71 comprises the amino acid sequence of SEQ ID NO: 122. In some embodiments, an isolated FN3 domain that binds CD71 comprises the amino acid sequence of SEQ ID NO: 123. In some embodiments, an isolated FN3 domain that binds CD71 comprises the amino acid sequence of SEQ ID NO: 124. In some embodiments, an isolated FN3 domain that binds CD71 comprises the amino acid sequence of SEQ ID NO: 125. In some embodiments, an isolated FN3 domain that binds CD71 comprises the amino acid sequence of SEQ ID NO: 126. In some embodiments, an isolated FN3 domain that binds CD71 comprises the amino acid sequence of SEQ ID NO: 127. In some embodiments, an isolated FN3 domain that binds CD71 comprises the amino acid sequence of SEQ ID NO: 128. In some embodiments, an isolated FN3 domain that binds CD71 comprises the amino acid sequence of SEQ ID NO: 129. In some embodiments, an isolated FN3 domain that binds CD71 comprises the amino acid sequence of SEQ ID NO: 130. In some embodiments, an isolated FN3 domain that binds CD71 comprises the amino acid sequence of SEQ ID NO: 131. In some embodiments, an isolated FN3 domain that binds CD71 comprises the amino acid sequence of SEQ ID NO: 132. In some embodiments, an isolated FN3 domain that binds CD71 comprises the amino acid sequence of SEQ ID NO: 133. In some embodiments, an isolated FN3 domain that binds CD71 comprises the amino acid sequence of SEQ ID NO: 134. In some embodiments, an isolated FN3 domain that binds CD71 comprises the amino acid sequence of SEQ ID NO: 135. In some embodiments, an isolated FN3 domain that binds CD71 comprises the amino acid sequence of SEQ ID NO: 136. In some embodiments, an isolated FN3 domain that binds CD71 comprises the amino acid sequence of SEQ ID NO: 137. In some embodiments, an isolated FN3 domain that binds CD71 comprises the amino acid sequence of SEQ ID NO: 138. In some embodiments, an isolated FN3 domain that binds CD71 comprises the amino acid sequence of SEQ ID NO: 139. In some embodiments, an isolated FN3 domain that binds CD71 comprises the amino acid sequence of SEQ ID NO: 140. In some embodiments, an isolated FN3 domain that binds CD71 comprises the amino acid sequence of SEQ ID NO: 141. In some embodiments, an isolated FN3 domain that binds CD71 comprises the amino acid sequence of SEQ ID NO: 142. In some embodiments, an isolated FN3 domain that binds CD71 comprises the amino acid sequence of SEQ ID NO: 143. In some embodiments, an isolated FN3 domain that binds CD71 comprises the amino acid sequence of SEQ ID NO: 144. In some embodiments, an isolated FN3 domain that binds CD71 comprises the amino acid sequence of SEQ ID NO: 145. In some embodiments, an isolated FN3 domain that binds CD71 comprises the amino acid sequence of SEQ ID NO: 146. In some embodiments, an isolated FN3 domain that binds CD71 comprises the amino acid sequence of SEQ ID NO: 147. In some embodiments, an isolated FN3 domain that binds CD71 comprises the amino acid sequence of SEQ ID NO: 148. In some embodiments, an isolated FN3 domain that binds CD71 comprises the amino acid sequence of SEQ ID NO: 149. In some embodiments, an isolated FN3 domain that binds CD71 comprises the amino acid sequence of SEQ ID NO: 150. In some embodiments, an isolated FN3 domain that binds CD71 comprises the amino acid sequence of SEQ ID NO: 151. In some embodiments, an isolated FN3 domain that binds CD71 comprises the amino acid sequence of SEQ ID NO: 152. In some embodiments, an isolated FN3 domain that binds CD71 comprises the amino acid sequence of SEQ ID NO: 153. In some embodiments, an isolated FN3 domain that binds CD71 comprises the amino acid sequence of SEQ ID NO: 154. In some embodiments, an isolated FN3 domain that binds CD71 comprises the amino acid sequence of SEQ ID NO: 155. In some embodiments, an isolated FN3 domain that binds CD71 comprises the amino acid sequence of SEQ ID NO: 156. In some embodiments, an isolated FN3 domain that binds CD71 comprises the amino acid sequence of SEQ ID NO: 157. In some embodiments, an isolated FN3 domain that binds CD71 comprises the amino acid sequence of SEQ ID NO: 158. In some embodiments, an isolated FN3 domain that binds CD71 comprises the amino acid sequence of SEQ ID NO: 159. In some embodiments, an isolated FN3 domain that binds CD71 comprises the amino acid sequence of SEQ ID NO: 160. In some embodiments, an isolated FN3 domain that binds CD71 comprises the amino acid sequence of SEQ ID NO: 161. In some embodiments, an isolated FN3 domain that binds CD71 comprises the amino acid sequence of SEQ ID NO: 162. In some embodiments, an isolated FN3 domain that binds CD71 comprises the amino acid sequence of SEQ ID NO: 163. In some embodiments, an isolated FN3 domain that binds CD71 comprises the amino acid sequence of SEQ ID NO: 164. In some embodiments, an isolated FN3 domain that binds CD71 comprises the amino acid sequence of SEQ ID NO: 165. In some embodiments, an isolated FN3 domain that binds CD71 comprises the amino acid sequence of SEQ ID NO: 166. In some embodiments, an isolated FN3 domain that binds CD71 comprises the amino acid sequence of SEQ ID NO: 167. In some embodiments, an isolated FN3 domain that binds CD71 comprises the amino acid sequence of SEQ ID NO: 168. In some embodiments, an isolated FN3 domain that binds CD71 comprises the amino acid sequence of SEQ ID NO: 169. In some embodiments, an isolated FN3 domain that binds CD71 comprises the amino acid sequence of SEQ ID NO: 170. In some embodiments, an isolated FN3 domain that binds CD71 comprises the amino acid sequence of SEQ ID NO: 171. In some embodiments, an isolated FN3 domain that binds CD71 comprises the amino acid sequence of SEQ ID NO: 172. In some embodiments, an isolated FN3 domain that binds CD71 comprises the amino acid sequence of SEQ ID NO: 173. In some embodiments, an isolated FN3 domain that binds CD71 comprises the amino acid sequence of SEQ ID NO: 174. In some embodiments, an isolated FN3 domain that binds CD71 comprises the amino acid sequence of SEQ ID NO: 175. In some embodiments, an isolated FN3 domain that binds CD71 comprises the amino acid sequence of SEQ ID NO: 176. In some embodiments, an isolated FN3 domain that binds CD71 comprises the amino acid sequence of SEQ ID NO: 177. In some embodiments, an isolated FN3 domain that binds CD71 comprises the amino acid sequence of SEQ ID NO: 178. In some embodiments, an isolated FN3 domain that binds CD71 comprises the amino acid sequence of SEQ ID NO: 179. In some embodiments, an isolated FN3 domain that binds CD71 comprises the amino acid sequence of SEQ ID NO: 180. In some embodiments, an isolated FN3 domain that binds CD71 comprises the amino acid sequence of SEQ ID NO: 181. In some embodiments, an isolated FN3 domain that binds CD71 comprises the amino acid sequence of SEQ ID NO: 182. In some embodiments, an isolated FN3 domain that binds CD71 comprises the amino acid sequence of SEQ ID NO: 183. In some embodiments, an isolated FN3 domain that binds CD71 comprises the amino acid sequence of SEQ ID NO: 184. In some embodiments, an isolated FN3 domain that binds CD71 comprises the amino acid sequence of SEQ ID NO: 185. In some embodiments, an isolated FN3 domain that binds CD71 comprises the amino acid sequence of SEQ ID NO: 186. In some embodiments, an isolated FN3 domain that binds CD71 comprises the amino acid sequence of SEQ ID NO: 187. In some embodiments, an isolated FN3 domain that binds CD71 comprises the amino acid sequence of SEQ ID NO: 188. In some embodiments, an isolated FN3 domain that binds CD71 comprises the amino acid sequence of SEQ ID NO: 189. In some embodiments, an isolated FN3 domain that binds CD71 comprises the amino acid sequence of SEQ ID NO: 190. In some embodiments, an isolated FN3 domain that binds CD71 comprises the amino acid sequence of SEQ ID NO: 191. In some embodiments, an isolated FN3 domain that binds CD71 comprises the amino acid sequence of SEQ ID NO: 192. In some embodiments, an isolated FN3 domain that binds CD71 comprises the amino acid sequence of SEQ ID NO: 193. In some embodiments, an isolated FN3 domain that binds CD71 comprises the amino acid sequence of SEQ ID NO: 194. In some embodiments, an isolated FN3 domain that binds CD71 comprises the amino acid sequence of SEQ ID NO: 195. In some embodiments, an isolated FN3 domain that binds CD71 comprises the amino acid sequence of SEQ ID NO: 196. In some embodiments, an isolated FN3 domain that binds CD71 comprises the amino acid sequence of SEQ ID NO: 197. In some embodiments, an isolated FN3 domain that binds CD71 comprises the amino acid sequence of SEQ ID NO: 198. In some embodiments, an isolated FN3 domain that binds CD71 comprises the amino acid sequence of SEQ ID NO: 199. In some embodiments, an isolated FN3 domain that binds CD71 comprises the amino acid sequence of SEQ ID NO: 200. In some embodiments, an isolated FN3 domain that binds CD71 comprises the amino acid sequence of SEQ ID NO: 201. In some embodiments, an isolated FN3 domain that binds CD71 comprises the amino acid sequence of SEQ ID NO: 202. In some embodiments, an isolated FN3 domain that binds CD71 comprises the amino acid sequence of SEQ ID NO: 203. In some embodiments, an isolated FN3 domain that binds CD71 comprises the amino acid sequence of SEQ ID NO: 204. In some embodiments, an isolated FN3 domain that binds CD71 comprises the amino acid sequence of SEQ ID NO: 205. In some embodiments, an isolated FN3 domain that binds CD71 comprises the amino acid sequence of SEQ ID NO: 206. In some embodiments, an isolated FN3 domain that binds CD71 comprises the amino acid sequence of SEQ ID NO: 207. In some embodiments, an isolated FN3 domain that binds CD71 comprises the amino acid sequence of SEQ ID NO: 208. In some embodiments, an isolated FN3 domain that binds CD71 comprises the amino acid sequence of SEQ ID NO: 209. In some embodiments, an isolated FN3 domain that binds CD71 comprises the amino acid sequence of SEQ ID NO: 210. In some embodiments, an isolated FN3 domain that binds CD71 comprises the amino acid sequence of SEQ ID NO: 211. In some embodiments, an isolated FN3 domain that binds CD71 comprises the amino acid sequence of SEQ ID NO: 212. In some embodiments, an isolated FN3 domain that binds CD71 comprises the amino acid sequence of SEQ ID NO: 213. In some embodiments, an isolated FN3 domain that binds CD71 comprises the amino acid sequence of SEQ ID NO: 214. In some embodiments, an isolated FN3 domain that binds CD71 comprises the amino acid sequence of SEQ ID NO: 215. In some embodiments, an isolated FN3 domain that binds CD71 comprises the amino acid sequence of SEQ ID NO: 216. In some embodiments, an isolated FN3 domain that binds CD71 comprises the amino acid sequence of SEQ ID NO: 217. In some embodiments, an isolated FN3 domain that binds CD71 comprises the amino acid sequence of SEQ ID NO: 218. In some embodiments, an isolated FN3 domain that binds CD71 comprises the amino acid sequence of SEQ ID NO: 219. In some embodiments, an isolated FN3 domain that binds CD71 comprises the amino acid sequence of SEQ ID NO: 220. In some embodiments, an isolated FN3 domain that binds CD71 comprises the amino acid sequence of SEQ ID NO: 221. In some embodiments, an isolated FN3 domain that binds CD71 comprises the amino acid sequence of SEQ ID NO: 222. In some embodiments, an isolated FN3 domain that binds CD71 comprises the amino acid sequence of SEQ ID NO: 223. In some embodiments, an isolated FN3 domain that binds CD71 comprises the amino acid sequence of SEQ ID NO: 224. In some embodiments, an isolated FN3 domain that binds CD71 comprises the amino acid sequence of SEQ ID NO: 225. In some embodiments, an isolated FN3 domain that binds CD71 comprises the amino acid sequence of SEQ ID NO: 226. In some embodiments, an isolated FN3 domain that binds CD71 comprises the amino acid sequence of SEQ ID NO: 227. In some embodiments, an isolated FN3 domain that binds CD71 comprises the amino acid sequence of SEQ ID NO: 228. In some embodiments, an isolated FN3 domain that binds CD71 comprises the amino acid sequence of SEQ ID NO: 229. In some embodiments, an isolated FN3 domain that binds CD71 comprises the amino acid sequence of SEQ ID NO: 230. In some embodiments, an isolated FN3 domain that binds CD71 comprises the amino acid sequence of SEQ ID NO: 231. In some embodiments, an isolated FN3 domain that binds CD71 comprises the amino acid sequence of SEQ ID NO: 232. In some embodiments, an isolated FN3 domain that binds CD71 comprises the amino acid sequence of SEQ ID NO: 233. In some embodiments, an isolated FN3 domain that binds CD71 comprises the amino acid sequence of SEQ ID NO: 234. In some embodiments, an isolated FN3 domain that binds CD71 comprises the amino acid sequence of SEQ ID NO: 235. In some embodiments, an isolated FN3 domain that binds CD71 comprises the amino acid sequence of SEQ ID NO: 236. In some embodiments, an isolated FN3 domain that binds CD71 comprises the amino acid sequence of SEQ ID NO: 237. In some embodiments, an isolated FN3 domain that binds CD71 comprises the amino acid sequence of SEQ ID NO: 238. In some embodiments, an isolated FN3 domain that binds CD71 comprises the amino acid sequence of SEQ ID NO: 239. In some embodiments, an isolated FN3 domain that binds CD71 comprises the amino acid sequence of SEQ ID NO: 240. In some embodiments, an isolated FN3 domain that binds CD71 comprises the amino acid sequence of SEQ ID NO: 241. In some embodiments, an isolated FN3 domain that binds CD71 comprises the amino acid sequence of SEQ ID NO: 242. In some embodiments, an isolated FN3 domain that binds CD71 comprises the amino acid sequence of SEQ ID NO: 243. In some embodiments, an isolated FN3 domain that binds CD71 comprises the amino acid sequence of SEQ ID NO: 244. In some embodiments, an isolated FN3 domain that binds CD71 comprises the amino acid sequence of SEQ ID NO: 245. In some embodiments, an isolated FN3 domain that binds CD71 comprises the amino acid sequence of SEQ ID NO: 246. In some embodiments, an isolated FN3 domain that binds CD71 comprises the amino acid sequence of SEQ ID NO: 247. In some embodiments, an isolated FN3 domain that binds CD71 comprises the amino acid sequence of SEQ ID NO: 248. In some embodiments, an isolated FN3 domain that binds CD71 comprises the amino acid sequence of SEQ ID NO: 249. In some embodiments, an isolated FN3 domain that binds CD71 comprises the amino acid sequence of SEQ ID NO: 250. In some embodiments, an isolated FN3 domain that binds CD71 comprises the amino acid sequence of SEQ ID NO: 251. In some embodiments, an isolated FN3 domain that binds CD71 comprises the amino acid sequence of SEQ ID NO: 252. In some embodiments, an isolated FN3 domain that binds CD71 comprises the amino acid sequence of SEQ ID NO: 253. In some embodiments, an isolated FN3 domain that binds CD71 comprises the amino acid sequence of SEQ ID NO: 254. In some embodiments, an isolated FN3 domain that binds CD71 comprises the amino acid sequence of SEQ ID NO: 255. In some embodiments, an isolated FN3 domain that binds CD71 comprises the amino acid sequence of SEQ ID NO: 256. In some embodiments, an isolated FN3 domain that binds CD71 comprises the amino acid sequence of SEQ ID NO: 257. In some embodiments, an isolated FN3 domain that binds CD71 comprises the amino acid sequence of SEQ ID NO: 258. In some embodiments, an isolated FN3 domain that binds CD71 comprises the amino acid sequence of SEQ ID NO: 259. In some embodiments, an isolated FN3 domain that binds CD71 comprises the amino acid sequence of SEQ ID NO: 260. In some embodiments, an isolated FN3 domain that binds CD71 comprises the amino acid sequence of SEQ ID NO: 261. In some embodiments, an isolated FN3 domain that binds CD71 comprises the amino acid sequence of SEQ ID NO: 262. In some embodiments, an isolated FN3 domain that binds CD71 comprises the amino acid sequence of SEQ ID NO: 263. In some embodiments, an isolated FN3 domain that binds CD71 comprises the amino acid sequence of SEQ ID NO: 264. In some embodiments, an isolated FN3 domain that binds CD71 comprises the amino acid sequence of SEQ ID NO: 265. In some embodiments, an isolated FN3 domain that binds CD71 comprises the amino acid sequence of SEQ ID NO: 266. In some embodiments, an isolated FN3 domain that binds CD71 comprises the amino acid sequence of SEQ ID NO: 267. In some embodiments, an isolated FN3 domain that binds CD71 comprises the amino acid sequence of SEQ ID NO: 268. In some embodiments, an isolated FN3 domain that binds CD71 comprises the amino acid sequence of SEQ ID NO: 269. In some embodiments, an isolated FN3 domain that binds CD71 comprises the amino acid sequence of SEQ ID NO: 270. In some embodiments, an isolated FN3 domain that binds CD71 comprises the amino acid sequence of SEQ ID NO: 271. In some embodiments, an isolated FN3 domain that binds CD71 comprises the amino acid sequence of SEQ ID NO: 272. In some embodiments, an isolated FN3 domain that binds CD71 comprises the amino acid sequence of SEQ ID NO: 273. In some embodiments, an isolated FN3 domain that binds CD71 comprises the amino acid sequence of SEQ ID NO: 274. In some embodiments, an isolated FN3 domain that binds CD71 comprises the amino acid sequence of SEQ ID NO: 275. In some embodiments, an isolated FN3 domain that binds CD71 comprises the amino acid sequence of SEQ ID NO: 276. In some embodiments, an isolated FN3 domain that binds CD71 comprises the amino acid sequence of SEQ ID NO: 277. In some embodiments, an isolated FN3 domain that binds CD71 comprises the amino acid sequence of SEQ ID NO: 278. In some embodiments, an isolated FN3 domain that binds CD71 comprises the amino acid sequence of SEQ ID NO: 279. In some embodiments, an isolated FN3 domain that binds CD71 comprises the amino acid sequence of SEQ ID NO: 280. In some embodiments, an isolated FN3 domain that binds CD71 comprises the amino acid sequence of SEQ ID NO: 281. In some embodiments, an isolated FN3 domain that binds CD71 comprises the amino acid sequence of SEQ ID NO: 282. In some embodiments, an isolated FN3 domain that binds CD71 comprises the amino acid sequence of SEQ ID NO: 283. In some embodiments, an isolated FN3 domain that binds CD71 comprises the amino acid sequence of SEQ ID NO: 284. In some embodiments, an isolated FN3 domain that binds CD71 comprises the amino acid sequence of SEQ ID NO: 285. In some embodiments, an isolated FN3 domain that binds CD71 comprises the amino acid sequence of SEQ ID NO: 286. In some embodiments, an isolated FN3 domain that binds CD71 comprises the amino acid sequence of SEQ ID NO: 287. In some embodiments, an isolated FN3 domain that binds CD71 comprises the amino acid sequence of SEQ ID NO: 288. In some embodiments, an isolated FN3 domain that binds CD71 comprises the amino acid sequence of SEQ ID NO: 289. In some embodiments, an isolated FN3 domain that binds CD71 comprises the amino acid sequence of SEQ ID NO: 290. In some embodiments, an isolated FN3 domain that binds CD71 comprises the amino acid sequence of SEQ ID NO: 291. In some embodiments, an isolated FN3 domain that binds CD71 comprises the amino acid sequence of SEQ ID NO: 292. In some embodiments, an isolated FN3 domain that binds CD71 comprises the amino acid sequence of SEQ ID NO: 293. In some embodiments, an isolated FN3 domain that binds CD71 comprises the amino acid sequence of SEQ ID NO: 294. In some embodiments, an isolated FN3 domain that binds CD71 comprises the amino acid sequence of SEQ ID NO: 295. In some embodiments, an isolated FN3 domain that binds CD71 comprises the amino acid sequence of SEQ ID NO: 296. In some embodiments, an isolated FN3 domain that binds CD71 comprises the amino acid sequence of SEQ ID NO: 297. In some embodiments, an isolated FN3 domain that binds CD71 comprises the amino acid sequence of SEQ ID NO: 298. In some embodiments, an isolated FN3 domain that binds CD71 comprises the amino acid sequence of SEQ ID NO: 299. In some embodiments, an isolated FN3 domain that binds CD71 comprises the amino acid sequence of SEQ ID NO: 300. In some embodiments, an isolated FN3 domain that binds CD71 comprises the amino acid sequence of SEQ ID NO: 301. In some embodiments, an isolated FN3 domain that binds CD71 comprises the amino acid sequence of SEQ ID NO: 302. In some embodiments, an isolated FN3 domain that binds CD71 comprises the amino acid sequence of SEQ ID NO: 303. In some embodiments, an isolated FN3 domain that binds CD71 comprises the amino acid sequence of SEQ ID NO: 304. In some embodiments, an isolated FN3 domain that binds CD71 comprises the amino acid sequence of SEQ ID NO: 305. In some embodiments, an isolated FN3 domain that binds CD71 comprises the amino acid sequence of SEQ ID NO: 306. In some embodiments, an isolated FN3 domain that binds CD71 comprises the amino acid sequence of SEQ ID NO: 307. In some embodiments, an isolated FN3 domain that binds CD71 comprises the amino acid sequence of SEQ ID NO: 308. In some embodiments, an isolated FN3 domain that binds CD71 comprises the amino acid sequence of SEQ ID NO: 309.

In some embodiments, the FN3 domain binds to human CD71 at site on CD71 that does not compete with transferrin binding to CD71. In some embodiments, the FN3 domain comprises a sequence of SEQ ID NO: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, or 149.

In some embodiments, the isolated FN3 domain that binds CD71 comprises an initiator methionine (Met) linked to the N-terminus of the molecule.

In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOs: 33-50. In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%9, 90%, 91%, 92%, 93%, 94%, 95%, 96%, 97%, 98% or 99% identical to one of the amino acid sequences of SEQ ID NOs: 51-61 or 62. In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOs: 81-309. In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOs: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, or 149.

Percent identity can be determined using the default parameters to align two sequences using BlastP available through the NCBI website.

Conjugates of the FN3 Domains that Bind CD71 of the Disclosure

In some embodiments, an isolated FN3 domain that binds CD71 conjugated to a heterologous molecule(s) is provided.

In some embodiments, the FN3 domain is conjugated to an oligonucleotide. For example, the oligonucleotide can be used for inhibiting the expression of a gene or mRNA transcript. The oligonucleotide can be a siRNA, miRNA, antisense oligonucleotide, and the like.

In some embodiments, the peptide is conjugated to a lipid nanoparticle, which can be used, for example, for cell-specific targeting.

In some embodiments, the protein is conjugated to a binding moiety that targets CD71 or another protein for protein degradation. For example, the protein can be conjugated to a PROTACS (binding moieties for an E3 ubiquitin ligase) and thus deliver the protein to the E3 ligase. These can linked through a linker, such as a glycine-serine linker and the like.

The FN3 domain that binds to CD71 can also be conjugated or linked to another FN3 domain that binds to a different target, other than CD71. This would enable the peptide to be multi-specific (e.g. bi-specific, tri-specific, etc.), such that it binds to CD71 and another, for example, protein. In some embodiments, the CD71 FN3 binding domain is linked to another FN3 domain that binds to an antigen expressed by a tumor cell (tumor antigen).

In some embodiments, FN3 domains can be linked together by a linker to form a bivalent FN3 domain. The linker can be a flexible linker. In some embodiments, the linker is a G/S linker. In some embodiments the linker has 1, 2, 3, or 4 G/S repeats. A G/S repeat unit is four glycines followed by a serine, e.g. GGGGS.

In some embodiments, the heterologous molecule is a detectable label or a therapeutic agent, such as, but not limited to a cytotoxic agent.

In some embodiments, an FN3 domain that binds CD71 conjugated to a detectable label is provided. Non-limiting examples of detectable labels are provided for herein.

In some embodiments, an FN3 domain that binds CD71 conjugated to a therapeutic agent is provided. Non-limiting examples of therapeutic agents, such as, but not limited to, cytotoxic agents, are provided for herein.

The FN3 domains that bind CD71 conjugated to a detectable label can be used to evaluate expression of CD71 on samples such as tumor tissue in vivo or in vitro.

Detectable labels include compositions that when conjugated to the FN3 domains that bind CD71 renders CD71 detectable, via spectroscopic, photochemical, biochemical, immunochemical, or other chemical methods.

Exemplary detectable labels include, but are not limited to, radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, cintillants, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In some embodiments, the detectable label emits a signal as a result of being stimulated by an external stimulus, such as a magnetic or electric, or electromagnetic field.

Exemplary radioactive isotopes may be γ-emitting, Auger-emitting, O-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e., iodine) to 83 (i.e., bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The FN3 domains that specifically bind CD71 conjugated to a detectable label may be used, for example, as an imaging agent to evaluate tumor distribution, diagnosis for the presence of tumor cells and/or, recurrence of tumor.

In some embodiments, the FN3 domains that specifically bind CD71 are conjugated to a therapeutic agent, such as, but not limited to, a cytotoxic agent.

In some embodiments, the therapeutic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The FN3 domains that bind CD71 conjugated to a therapeutic agent disclosed herein may be used in the targeted delivery of the therapeutic agent to CD71 expressing cells (e.g. tumor cells), and intracellular accumulation therein. Although not bound to any particular theory, this type of delivery can be helpful where systemic administration of these unconjugated agents may result in unacceptable levels of toxicity to normal cells.

In some embodiments, the therapeutic agent can elicit their cytotoxic and/or cytostatic effects by mechanisms such as, but not limited to, tubulin binding, DNA binding, topoisomerase inhibition, DNA cross linking, chelation, spliceosome inhibition, NAMPT inhibition, and HDAC inhibition.

In some embodiments, the therapeutic agent is a spliceosome inhibitor, a NAMPT inhibitor, or a HDAC inhibitor. In some embodiments, the agent is an immune system agonist, for example, TLR7, 8, 9, RIG-I (dsRNA), and STING (CpG) agonists. In some embodiments, the agent is daunomycin, doxorubicin, methotrexate, vindesine, bacterial toxins such as diphtheria toxin, ricin, geldanamycin, maytansinoids or calicheamicin.

In some embodiments, the therapeutic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleuritesfordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, or the tricothecenes.

In some embodiments, the therapeutic agent is a radionuclide, such as ²¹²Bi, ¹³¹I, ¹³¹In, ⁹⁰Y, or ¹⁸⁶Re.

In some embodiments, the therapeutic agent is dolastatin or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancerand antifungal activity. The dolastatin or auristatin drug moiety may be attached to the FN3 domain through the N (amino) terminus or the C (carboxyl) terminus of the peptidic drug moiety (WO 02/088172), or via any cysteine engineered into the FN3 domain.

In some embodiments, therapeutic agent can be, for example, auristatins, camptothecins, duocarmycins, etoposides, maytansines and maytansinoids, taxanes, benzodiazepines or benzodiazepine containing drugs (e.g., pyrrolo[1,4]-benzodiazepines (PBDs), indolinobenzodiazepines, and oxazolidinobenzodiazepines) or vinca alkaloids.

The FN3 domains that specifically bind CD71 may be conjugated to a detectable label using known methods.

In some embodiments, the detectable label is complexed with a chelating agent.

In some embodiments, the detectable label is conjugated to the FN3 domain that binds CD71 via a linker.

The detectable label, therapeutic compound, or the cytotoxic compound may be linked directly, or indirectly, to the FN3 domain that binds CD71 using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10, tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis(p-diazoniumbenzoyl)ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiment, the FN3 domain that binds CD71 is removed from the blood via renal clearance.

Isolation of CD71 binding FN3 domains from a library based on Tencon sequence Tencon (SEQ ID NO: 1) is a non-naturally occurring fibronectin type III (FN3) domain designed from a consensus sequence of fifteen FN3 domains from human tenascin-C (Jacobs et al., Protein Engineering, Design, and Selection, 25:107-117, 2012; U.S. Pat. Publ. No. 2010/0216708). The crystal structure of Tencon shows six surface-exposed loops that connect seven beta-strands as is characteristic to the FN3 domains, the beta-strands referred to as A, B, C, D, E, F, and G, and the loops referred to as AB, BC, CD, DE, EF, and FG loops (Bork and Doolittle, Proc Natl Acad Sci USA 89:8990-8992, 1992; U.S. Pat. No. 6,673,901). These loops, or selected residues within each loop, may be randomized in order to construct libraries of fibronectin type III (FN3) domains that may be used to select novel molecules that bind CD71. Table 1 shows positions and sequences of each loop and beta-strand in Tencon (SEQ ID NO: 1).

Library designed based on Tencon sequence may thus have randomized FG loop, or randomized BC and FG loops, such as libraries TCL1 or TCL2 as described below. The Tencon BC loop is 7 amino acids long, thus 1, 2, 3, 4, 5, 6 or 7 amino acids may be randomized in the library diversified at the BC loop and designed based on Tencon sequence. The Tencon FG loop is 7 amino acids long, thus 1, 2, 3, 4, 5, 6 or 7 amino acids may be randomized in the library diversified at the FG loop and designed based on Tencon sequence. Further diversity at loops in the Tencon libraries may be achieved by insertion and/or deletions of residues at loops. For example, the FG and/or BC loops may be extended by 1-22 amino acids, or decreased by 1-3 amino acids. The FG loop in Tencon is 7 amino acids long, whereas the corresponding loop in antibody heavy chains ranges from 4-28 residues. To provide maximum diversity, the FG loop may be diversified in sequence as well as in length to correspond to the antibody CDR3 length range of 4-28 residues. For example, the FG loop can further be diversified in length by extending the loop by additional 1, 2, 3, 4 or 5 amino acids.

Library designed based on Tencon sequence may also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface). A library design based on Tencon alternative C-CD-F-FG surface is described in U.S. Pat. Publ. No. 2013/0226834. Library designed based on Tencon sequence also includes libraries designed based on Tencon variants, such as Tencon variants having substitutions at residues positions 11, 14, 17, 37, 46, 73, or 86 (residue numbering corresponding to SEQ ID NO: 1), and which variants display improve thermal stability. Exemplary Tencon variants are described in US Pat. Publ. No. 2011/0274623, and include Tencon27 (SEQ ID NO: 4) having substitutions E11R, L17A, N46V and E861 when compared to Tencon of SEQ ID NO: 1.

TABLE 1
Tencon topology

| FN3 | Tencon |
|---|---|
| domain | (SEQ ID NO: 1) |
| A strand | 1-12 |
| AB loop | 13-16 |
| B strand | 17-21 |
| BC loop | 22-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-81 |
| G strand | 82-89 |

Tencon and other FN3 sequence based libraries may be randomized at chosen residue positions using a random or defined set of amino acids. For example, variants in the library having random substitutions may be generated using NNK codons, which encode all 20 naturally occurring amino acids. In other diversification schemes, DVK codons may be used to encode amino acids Ala, Trp, Tyr, Lys, Thr, Asn, Lys, Ser, Arg, Asp, Glu, Gly, and Cys. Alternatively, NNS codons may be used to give rise to all 20 amino acid residues and simultaneously reducing the frequency of stop codons. Libraries of FN3 domains with biased amino acid distribution at positions to be diversified may be synthesized for example using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well-known IUB code.

The FN3 domains that specifically bind CD71 may be isolated by producing the FN3 library such as the Tencon library using cis display to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476; Odegrip et al., Proc Natl Acad Sci USA 101, 2806-2810, 2004), and assaying the library for specific binding to PSMA by any method known in the art and described in the Example. Exemplary well known methods which can be used are ELISA, sandwich immunoassays, and competitive and non-competitive assays (see, e.g., Ausubel et al., eds, 1994, Current Protocols in Molecular Biology, Vol. 1, John Wiley & Sons, Inc., New York). The identified FN3 domains that specifically bind CD71 are further characterized for their binding to CD71, modulation of CD71 activity, internalization, stability, and other desired characteristics.

The FN3 domains that specifically bind CD71 may be generated using any FN3 domain as a template to generate a library and screening the library for molecules specifically binding CD71 using methods provided within. Exemplar FN3 domains that may be used are the 3rd FN3 domain of tenascin C (TN3), Fibcon, and the 10^{th} FN3 domain of fibronectin (FN10). Accordingly, PCT applications WO 2010/051274, WO 2011/137319, and WO 2013/049275 are incorporated herein in their entirety. Standard cloning and expression techniques are used to clone the libraries into a vector or synthesize double stranded cDNA cassettes of the library, to express, or to translate the libraries in vitro. For example ribosome display (Hanes and Pluckthun, Proc Natl Acad Sci USA, 94, 4937-4942, 1997), mRNA display (Roberts and Szostak, Proc Natl Acad Sci USA, 94, 12297-12302, 1997), or other cell-free systems (U.S. Pat. No. 5,643,768) can be used. The libraries of the FN3 domain variants may be expressed as fusion proteins displayed on the surface for example of any suitable bacteriophage. Methods for displaying fusion polypeptides on the surface of a bacteriophage are well known (U.S. Pat. Publ. No. 2011/0118144; Int. Pat. Publ. No. WO2009/085462; U.S. Pat. Nos. 6,969,108; 6,172,197; 5,223,409; 6,582,915; 6,472,147).

In some embodiments, the FN3 domain that binds CD71 is based on Tencon sequence of SEQ ID NO: 1 or Tencon27 sequence of SEQ ID NO: 4, the SEQ ID NO: 1 or the SEQ ID NO: 4, optionally having substitutions at residues positions 11, 14, 17, 37, 46, 73, and/or 86.

In some embodiments, the FN3 protein or polypeptide is one that binds to human CD71 at a site on CD71 that does not compete with transferrin binding to CD71. As used herein, a site on CD71 that does not compete with transferrin binding to CD71 refers to an epitope or part of CD71 where the binding of the FN3 protein does not compete or inhibit the binding of transferrin to CD71. The competition, or lack thereof, can be complete or partial. In some embodiments, the binding also does not inhibit the internalization of transferrin into the cell through its interaction with CD71.

In some embodiments, methods for identifying a FN3 protein that binds to CD71 at a site that does not compete or inhibit transferrin binding to CD71 are provided. In some embodiments, the methods comprise contacting CD71 in the presence of transferrin or an agent that binds to the CD71 transferrin binding site with a test FN3 protein; and identifying a test FN3 protein that binds to CD71 in the presence of transferrin or an agent that binds to the CD71 transferrin binding site. In some embodiments, the method comprises isolating the test FN3 protein that binds to CD71 in the presence of transferrin or an agent that binds to the CD71 transferrin binding site. In some embodiments, the methods comprise sequencing the test FN3 protein that binds to CD71 in the presence of transferrin or an agent that binds to the CD71 transferrin binding site. In some embodiments, the methods comprise preparing or obtaining a nucleic acid sequence encoding the test FN3 protein that binds to CD71 in the presence of transferrin or an agent that binds to the CD71 transferrin binding site. In some embodiments, the methods comprise expressing the test FN3 protein that binds to CD71 in the presence of transferrin or an agent that binds to the CD71 transferrin binding site from a nucleic acid sequence encoding the test FN3 protein that binds to CD71 in the presence of transferrin or an agent that binds to the CD71 transferrin binding site. In some embodiments, the test FN3 protein is expressed in a cell. In some embodiments, the methods comprise isolating and/or purifying the expressed test FN3 protein.

In some embodiments a FN3 protein is provided, wherein the FN3 protein is identified according to any method provided herein.

The FN3 domains that specifically bind CD71 may be modified to improve their properties such as improve thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences (Lehmann and Wyss, Curr. Opin. Biotechnol., 12, 371-375, 2001). High thermal stability may increase the yield of the expressed protein, improve solubility or activity, decrease immunogenicity, and minimize the need of a cold chain in manufacturing. Residues that may be substituted to improve thermal stability of Tencon (SEQ ID NO: 1) are residue positions 11, 14, 17, 37, 46, 73, or 86, and are described in US Pat. Publ. No. 2011/0274623. Substitutions corresponding to these residues may be incorporated to the FN3 domain containing molecules disclosed herein.

Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“Tₘ”) temperature, the temperature in ° Celsius (° C.) at which half of the molecules become unfolded, using standard methods. Typically, the higher the Tₘ, the more stable the molecule. In addition to heat, the chemical environment also changes the ability of the protein to maintain a particular three dimensional structure.

In some embodiments, the FN3 domain that binds CD71 may exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the Tₘ.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate, lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH₃COOH), halogenated acetic acids), hydrophobic molecules (e.g. phospholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

The FN3 domain that binds CD71 may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. Exemplary linker include (GS)₂, (SEQ ID NO: 63), (GGGS)₂ (SEQ ID NO: 64), (GGGGS)₅ (SEQ ID NO: 65), (AP)₂ (SEQ ID NO: 66), (AP)₅ (SEQ ID NO: 67), (AP)₁₀ (SEQ ID NO: 68), (AP)₂₀ (SEQ ID NO: 69) and A(EAAAK)₅AAA (SEQ ID NO: 70). The dimers and multimers may be linked to each other in a N- to C-direction. The use of naturally occurring as well as artificial peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456).

### Half-Life Extending Moieties

The FN3 domains that specifically bind CD71 may incorporate other subunits for example via covalent interaction. In some embodiments, the FN3 domains that specifically bind CD71 further comprise a half-life extending moiety. Exemplary half-life extending moieties are albumin, albumin variants, albumin-binding proteins and/or domains, transferrin and fragments and analogues thereof, and Fc regions. Amino acid sequences of the human Fc regions are well known, and include IgG1, IgG2, IgG3, IgG4, IgM, IgA and IgE Fc regions. In some embodiments, the FN3 domains that specifically bind CD71 may incorporate a second FN3 domain that binds to a molecule that extends the half-life of the entire molecule, such as, but not limited to, any of the half-life extending moieties described herein. In some embodiments, the second FN3 domain binds to albumin, albumin variants, albumin-binding proteins and/or domains, and fragments and analogues thereof.

All or a portion of an antibody constant region may be attached to the FN3 domain that binds CD71 to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor; BCR), and may be further modified by modifying residues in the Fc responsible for these activities (for review; see Strohl, Curr Opin Biotechnol. 20, 685-691, 2009).

Additional moieties may be incorporated into the FN3 domains that specifically bind CD71 such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules disclosed herein.

A pegyl moiety may for example be added to the FN3 domain that binds CD71 by incorporating a cysteine residue to the C-terminus of the molecule, or engineering cysteines into residue positions that face away from the CD71 binding face of the molecule, and attaching a pegyl group to the cysteine using well known methods.

FN3 domains that specifically bind CD71 incorporating additional moieties may be compared for functionality by several well-known assays. For example, altered properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules disclosed herein in in vivo models.

### Polynucleotides, Vectors, Host Cells

In some embodiments, nucleic acids encoding the FN3 domains specifically binding CD71 as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof are provided. Certain exemplary polynucleotides are disclosed herein, however, other polynucleotides which, given the degeneracy of the genetic code or codon preferences in a given expression system, encode the FN3 domains disclosed herein are also within the scope of the disclosure.

In some embodiments, an isolated polynucleotide encodes the FN3 domain specifically binding CD71 comprising the amino acid sequence of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309.

The polynucleotides disclosed herein may be produced by chemical synthesis such as solid phase polynucleotide synthesis on an automated polynucleotide synthesizer and assembled into complete single or double stranded molecules. Alternatively, the polynucleotides disclosed herein may be produced by other techniques such as PCR followed by routine cloning. Techniques for producing or obtaining polynucleotides of a given known sequence are well known in the art.

The polynucleotides disclosed herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

In some embodiments, a vector comprising at least one polynucleotide disclosed herein is provided. Such vectors may be plasmid vectors, viral vectors, vectors for baculovirus expression, transposon based vectors or any other vector suitable for introduction of the polynucleotides disclosed herein into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

In some embodiments, a host cell comprising the vector is provided. The FN3 domain that specifically bind CD71 may be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N Y (1989); Harlow and Lane, Antibodies, a Laboratory Manual, Cold Spring Harbor, N Y (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21 (DE3), BL21-GOLD (DE3), XL1-Blue, JM109, HMS174, HMS174 (DE3), and any of the natural or engineered E. coli spp, Klebsiella spp., or Pseudomonas spp strains.

In some embodiments, a method of producing the isolated FN3 domain that binds CD71, comprising culturing the isolated host cell under conditions such that the isolated FN3 domain that binds CD71 is expressed, and purifying the FN3 domain.

The FN3 domains that bind CD71 may be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography, or high performance liquid chromatography (HPLC).

### Anti-Idiotypic Antibodies

In some embodiments, an anti-idiotypic antibody binds to the FN3 domain.

In some embodiments, an anti-idiotypic antibody that binds the FN3 domain comprises the amino acid sequences of one of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309.

### Kits

In some embodiments, a kit comprising the FN3 domain that binds CD71 is provided.

The kit may be used for therapeutic uses and as a diagnostic kit.

In some embodiments, the kit comprises the FN3 domain that binds CD71 and reagents for detecting the FN3 domain. In some embodiments, the kit comprises a bivalent FN3 domain. The kit can include one or more other elements including: instructions for use; other reagents, e.g., a label, an agent useful for chelating, or otherwise coupling, a radioprotective composition; devices or other materials for preparing the FN3 domain that binds CD71 for administration for imaging, diagnostic or therapeutic purpose; pharmaceutically acceptable carriers; and devices or other materials for administration to a subject.

In some embodiments, the kit comprises the FN3 domain that binds CD71 comprising the amino acid sequences of one of SEQ ID NOs: 33-50.

In some embodiments, the kit comprises the FN3 domain that binds CD71 comprising the amino acid sequences of one of SEQ ID NOs: 51-61.

In some embodiments, the kit comprises the FN3 domain that binds CD71 comprising the amino acid sequences of one of SEQ ID NOs: 81-309.

In some embodiments, the kit comprises the FN3 domain that binds CD71 comprising the amino acid sequences of one of SEQ ID NOs: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, or 149.

### Uses of CD71 Binding FN3 Domains

The FN3 domains that specifically bind CD71 or conjugates thereof may be used to diagnose, monitor, modulate, treat, alleviate, help prevent the incidence of, or reduce the symptoms of human disease or specific pathologies in cells, tissues, organs, fluid, or, generally, a host.

In some embodiments, the FN3 domains that specifically bind CD71 or conjugates thereof may also be used in imaging CD71 positive tumor tissue in a subject.

The methods disclosed herein may be used with an animal patient belonging to any classification. Examples of such animals include mammals such as humans, rodents, dogs, cats and farm animals.

In some embodiments, a method of diagnosing a subject having, or who is likely to develop cancer of a tissue based on the expression of CD71 by cells of the cancer tissue, methods of predicting success of immunotherapy, methods of prognosis, and methods of treatment are provided.

In some embodiments, a method of detecting CD71-expressing cancer cells in a tumor tissue is provided, the method comprising: obtaining a sample of the tumor tissue from a subject; detecting whether CD71 is expressed in the tumor tissue by contacting toe sample of the tumor tissues with the FN3 domain that binds CD71 comprising the amino acid sequence of one of SEQ ID NOs: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309, and detecting the binding between CD71 and the FN3 domain.

In some embodiments, the CD71 cell is a cell involved in a CNS diseases, inflammatory/immune diseases, such as MS & infectious diseases of the brain.

In some embodiments, the tissue can be tissue of any organ or anatomical system, that expresses CD71.

In some embodiments, CD71 expression may be evaluated using known methods, such as immunohistochemistry or ELISA.

In some embodiments, a method of isolating CD71 expressing cells is provided, the method comprising: obtaining a sample from a subject; contacting the sample with the FN3 domain that binds CD71 comprising the amino acid sequence of one of SEQ ID NOs: 33-50, and isolating the cells bound to the FN3 domains.

In some embodiments, a method of isolating CD71 expressing cells is provided, the method comprising: obtaining a sample from a subject; contacting the sample with the FN3 domain that binds CD71 comprising the amino acid sequence of one of SEQ ID NOs: 51-61, and isolating the cells bound to the FN3 domains.

In some embodiments, a method of detecting CD71-expressing cancer cells in a tumor tissue is provided, the method comprising: conjugating the FN3 domain that binds CD71 comprising the amino acid sequence of one of SEQ ID NOs: 33-50 to a detectable label to form a conjugate; administering the conjugate to a subject; and visualizing the CD71 expressing cancer cells to which the conjugate is bound.

In some embodiments, a method of detecting CD71-expressing cancer cells in a tumor tissue is provided, the method comprising: conjugating the FN3 domain that binds CD71 comprising the amino acid sequence of one of SEQ ID NOs: 51-62 or 81-309 to a detectable label to form a conjugate; administering the conjugate to a subject; and visualizing the CD71 expressing cancer cells to which the conjugate is bound.

In some embodiments, a method of treating a subject having cancer is provided, the method comprising administering to the subject a FN3 domain that binds CD71. In some embodiments, the FN3 domain is conjugated to a therapeutic agent (e.g. cytotoxic agent, an oligonucleotide, a FN3 domain that binds to another target, and the like).

In some embodiments, the subject has a solid tumor.

In some embodiments, the solid tumor is a melanoma.

In some embodiments, the solid tumor is a lung cancer. In some embodiments, the solid tumor is a non-small cell lung cancer (NSCLC). In some embodiments, the solid tumor is a squamous non-small cell lung cancer (NSCLC). In some embodiments, the solid tumor is a non-squamous NSCLC. In some embodiments, the solid tumor is a lung adenocarcinoma.

In some embodiments, the solid tumor is a renal cell carcinoma (RCC).

In some embodiments, the solid tumor is a mesothelioma.

In some embodiments, the solid tumor is a nasopharyngeal carcinoma (NPC).

In some embodiments, the solid tumor is a colorectal cancer.

In some embodiments, the solid tumor is a prostate cancer. In some embodiments, the solid tumor is castration-resistant prostate cancer.

In some embodiments, the solid tumor is a stomach cancer.

In some embodiments, the solid tumor is an ovarian cancer.

In some embodiments, the solid tumor is a gastric cancer.

In some embodiments, the solid tumor is a liver cancer.

In some embodiments, the solid tumor is pancreatic cancer.

In some embodiments, the solid tumor is a thyroid cancer.

In some embodiments, the solid tumor is a squamous cell carcinoma of the head and neck.

In some embodiments, the solid tumor is a carcinomas of the esophagus or gastrointestinal tract.

In some embodiments, the solid tumor is a breast cancer.

In some embodiments, the solid tumor is a fallopian tube cancer.

In some embodiments, the solid tumor is a brain cancer.

In some embodiments, the solid tumor is an urethral cancer.

In some embodiments, the solid tumor is a genitourinary cancer.

In some embodiments, the solid tumor is an endometriosis.

In some embodiments, the solid tumor is a cervical cancer.

In some embodiments, the solid tumor is a metastatic lesion of the cancer.

In some embodiments, the subject has a hematological malignancy.

In some embodiments, the hematological malignancy is a lymphoma, a myeloma or a leukemia. In some embodiments, the hematological malignancy is a B cell lymphoma. In some embodiments, the hematological malignancy is Burkitt's lymphoma. In some embodiments, the hematological malignancy is Hodgkin's lymphoma. In some embodiments, the hematological malignancy is a non-Hodgkin's lymphoma.

In some embodiments, the hematological malignancy is a myelodysplastic syndrome.

In some embodiments, the hematological malignancy is an acute myeloid leukemia (AML). In some embodiments, the hematological malignancy is a chronic myeloid leukemia (CML). In some embodiments, the hematological malignancy is a chronic myelomoncytic leukemia (CMML).

In some embodiments, the hematological malignancy is a multiple myeloma (MM).

In some embodiments, the hematological malignancy is a plasmacytoma.

In some embodiments, the compositions or pharmaceutical compositions provided herein may be administered alone or in combination with other therapeutics, that is, simultaneously or sequentially. In some embodiments, the other or additional therapeutics are other anti-tumor agent or therapeutics. Different tumor types and stages of tumors can require the use of various auxiliary compounds useful for treatment of cancer. For example, the compositions provided herein can be used in combination with various chemotherapeutics such as taxol, tyrosine kinase inhibitors, leucovorin, fluorouracil, irinotecan, phosphatase inhibitors, MEK inhibitors, among others. The composition may also be used in combination with drugs which modulate the immune response to the tumor such as anti-PD-1 or anti-CTLA-4, among others. Additional treatments can be agents that modulate the immune system, such antibodies that target PD-1 or PD-L1.

In some embodiments, the FN3 domains that specifically bind CD71 or conjugates thereof that may be used to diagnose, monitor, modulate, treat, alleviate, help prevent the incidence of, or reduce the symptoms of human disease or specific pathologies in cells, tissues, organs, fluid, or, generally, a host, also exhibit the property of being able to cross the blood brain barrier. The blood-brain barrier (BBB) prevents most macromolecules (e.g., DNA, RNA, and polypeptides) and many small molecules from entering the brain. The BBB is principally composed of specialized endothelial cells with highly restrictive tight junctions, consequently, passage of substances, small and large, from the blood into the central nervous system is controlled by the BBB. This structure makes treatment and management of patients with neurological diseases and disorders (e.g., brain cancer) difficult as many therapeutic agents cannot be delivered across the BBB with desirable efficiency. Additional conditions that involve disruptions of the BBB include: stroke, diabetes, seizures, hypertensive encephalopathy, acquired immunodeficiency syndrome, traumatic brain injuries, multiple sclerosis, Parkinson's disease (PD) and Alzheimer disease. This ability is especially useful for treating brain cancers including for example: astrocytoma, medulloblastoma, glioma, ependymoma, germinoma (pinealoma), glioblastoma multiform, oligodendroglioma, schwannoma, retinoblastoma, and congenital tumors; or a cancer of the spinal cord, e.g., neurofibroma, meningioma, glioma, and sarcoma. In certain embodiments, the FN3 domains that specifically bind CD71 comprising the amino acid sequence of one of SEQ ID NOs: 33-50 or conjugates thereof, are useful to deliver a therapeutic or cytotoxic agent, for example, across the blood brain barrier. In certain embodiments, the FN3 domains that specifically bind CD71 comprising the amino acid sequence of one of SEQ ID NOs: 51-61 or conjugates thereof, are useful to deliver a therapeutic or cytotoxic agent, for example, across the blood brain barrier. In some embodiments, the protein comprises a sequence of SEQ ID NOs: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, 149, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 81-309.

In some embodiments, the polypeptide that can facilitates the transport of a therapeutic across the BBB is a protein comprising a sequence of SEQ ID NO: 146, 214, 104, 259, 134, 92, 302, 235, 237, 152, 238, 136, 197, 212, 296, 226, 261, 307, 115, 112, 278, 297, 96, 222, 95, 233, 217, 252, 194, 164, 168, 174, 190, 257, 303, 284, 85, or 149.

“Treat” or “treatment” refers to the therapeutic treatment and prophylactic measures, wherein the object is to prevent or slow down (lessen) an undesired physiological change or disorder, such as the development or spread of cancer. In some embodiments, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the FN3 domains that specifically bind CD71 may vary according to factors such as the disease state, age, sex, and weight of the individual. Exemplary indicators of an effective FN3 domain that binds CD71 is improved well-being of the patient, decrease or shrinkage of the size of a tumor, arrested or slowed growth of a tumor, and/or absence of metastasis of cancer cells to other locations in the body.

### Administration/Pharmaceutical Compositions

In some embodiments, pharmaceutical compositions of the FN3 domains that specifically bind CD71, optionally conjugated to a detectable label, therapeutic, or a cytotoxic agent disclosed herein and a pharmaceutically acceptable carrier, are provided. For therapeutic use, the FN3 domains that specifically bind CD71 may be prepared as pharmaceutical compositions containing an effective amount of the domain or molecule as an active ingredient in a pharmaceutically acceptable carrier. “Carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules disclosed herein in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the FN3 domains disclosed herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intra-articular, intrabronchial, intra-abdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardial, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### EXAMPLES

The following examples are illustrative of the embodiments disclosed herein. These examples are provided for the purpose of illustration only and the embodiments should in no way be construed as being limited to these examples, but rather should be construed to encompass any and all variations which become evidence as a result of the teaching provided herein. Those of skill in the art will readily recognize a variety of non-critical parameters that could be changed or modified to yield essentially similar results.

### Example 1. Construction of Tencon Libraries with Randomized Loops

Tencon (SEQ ID NO: 1) is an immunoglobulin-like scaffold, fibronectin type III (FN3) domain, designed from a consensus sequence of fifteen FN3 domains from human tenascin-C (Jacobs et al., Protein Engineering, Design, and Selection, 25:107-117, 2012; U.S. Pat. No. 8,278,419). The crystal structure of Tencon shows six surface-exposed loops that connect seven beta-strands. These loops, or selected residues within each loop, can be randomized in order to construct libraries of fibronectin type III (FN3) domains that can be used to select novel molecules that bind to specific targets.

### Tencon:

(SEQ ID NO: 1)
LPAPKNLVVSEVTEDSLRLSWTAPDAAFDSFLIQYQESEKVGEAINLTV
PGSERSYDLTGLKPGTEYTVSIYGVKGGHRSNPLSAEFTT

Various libraries were generated using the Tencon scaffold and various design strategies. In general, libraries TCL1 and TCL2 produced good binders. Generation of TCL1 and TCL2 libraries are described in detail in Int. Pat. Publ. No. WO/2014081944A2.

### Construction of TCL1 Library

A library designed to randomize only the FG loop of Tencon (SEQ ID NO: 1), TCL1, was constructed for use with the cis-display system (Jacobs et al., Protein Engineering, Design, and Selection, 25:107-117, 2012). In this system, a single-strand DNA incorporating sequences for a Tac promoter, Tencon library coding sequence, RepA coding sequence, cis-element, and ori element is produced. Upon expression in an in vitro transcription/translation system, a complex is produced of the Tencon-RepA fusion protein bound in cis to the DNA from which it is encoded. Complexes that bind to a target molecule are then isolated and amplified by polymerase chain reaction (PCR), as described below.

Construction of the TCL1 library for use with cis-display was achieved by successive rounds of PCR to produce the final linear, double-stranded DNA molecules in two halves; the 5′ fragment contains the promoter and Tencon sequences, while the 3′ fragment contains the repA gene and the cis- and on elements. These two halves are combined by restriction digest in order to produce the entire construct. The TCL1 library was designed to incorporate random amino acids only in the FG loop of Tencon. NNS codons were used in the construction of this library, resulting in the possible incorporation of all 20 amino acids and one stop codon into the FG loop. The TCL1 library contains six separate sub-libraries, each having a different randomized FG loop length, from 7 to 12 residues, in order to further increase diversity.

TCL1 library
(SEQ ID NO: 2)
LPAPKNLVVSEVTEDSLRLSWTAPDAAFDSFLIQYQESEKVGEAI
NLTVPGSERSYDLTGLKPGTEYTVSIYGVX₇₋₁₂PLSAEFTT;

wherein
X₁, X₂, X₃, X₄, X₅, X₆, X₇ is any amino acid; and
X₈, X₉, X₁₀, X₁₁ and X₁₂ are any amino acid or deleted

### Construction of TCL2 Library

TCL2 library was constructed in which both the BC and the FG loops of Tencon were randomized and the distribution of amino acids at each position was strictly controlled. Table 2 shows the amino acid distribution at desired loop positions in the TCL2 library. The designed amino acid distribution had two aims. First, the library was biased toward residues that were predicted to be structurally important for Tencon folding and stability based on analysis of the Tencon crystal structure and/or from homology modeling. For example, position 29 was fixed to be only a subset of hydrophobic amino acids, as this residue was buried in the hydrophobic core of the Tencon fold. A second layer of design included biasing the amino acid distribution toward that of residues preferentially found in the heavy chain HCDR3 of antibodies, to efficiently produce high-affinity binders (Birtalan et al., J Mol Biol 377:1518-28, 2008; Olson et al., Protein Sci 16:476-84, 2007). Towards this goal, the “designed distribution” in Table 2 refers to the distribution as follows: 6% alanine, 6% arginine, 3.9% asparagine, 7.5% aspartic acid, 2.5% glutamic acid, 1.5% glutamine, 15% glycine, 2.3% histidine, 2.5% isoleucine, 5% leucine, 1.5% lysine, 2.5% phenylalanine, 4% proline, 10% serine, 4.5% threonine, 4% tryptophan, 17.3% tyrosine, and 4% valine. This distribution is devoid of methionine, cysteine, and STOP codons.

TCL2 library
(SEQ ID NO: 3)
LPAPKNLVVSEVTEDSLRLSWX₁X₂X₃X₄X₅X₆X₇X₈SFLIQYQES
EKVGEAINLTVPGSERSYDLTGLKPGTEYTVSIYGVX₉X₁₀X₁₁X₁₂
X₁₃SX₁₄X₁₅LSAEFTT;

wherein
X₁ is Ala, Arg, Asn, Asp, Glu, Gln, Gly, His, Ile, Leu, Lys, Phe, Pro, Ser, T Dr or Val;
X₂ is Ala, Arg, Asn, Asp, Glu, Gln, Gly, His, Ile, Leu, Lys, Phe, Pro, Ser, Thr, Trp, Tyr or Val;
X₃ Ala, Arg, Asn, Asp, Glu, Gln, Gly, His, Ile, Leu, Lys, Phe, Pro, Ser, Thr, Trp, Tyr or Val;
X₄ is Ala, Arg, Asn, Asp, Glu, Gln, Gly, His, Ile, Leu, Lys, Phe, Pro, Ser, Thr, Trp, Tyr or Val;
X₅ is Ala, Arg, Asn, Asp, Glu, Gln, Gly, His, Ile, Leu, Lys, Phe, Pro, Ser, Thr, Trp, Tyr or Val;
X₆ is Ala, Arg, Asn, Asp, Glu, Gln, Gly, His, Ile, Leu, Lys, Phe, Pro, Ser, Thr, Trp, Tyr or Val;
X₇ is Phe, Ile, Leu, Val or Tyr;
X₈ is Asp, Glu or Thr;
X₉ is Ala, Arg, Asn, Asp, Glu, Gln, Gly, His, Ile, Leu, Lys, Phe, Pro, Ser, Thr, Trp, Tyr or Val;
X₁₀ is Ala, Arg, Asn, Asp, Glu, Gln, Gly, His, Ile, Leu, Lys, Phe, Pro, Ser, Thr, Trp, Tyr or Val;
X₁₁ is Ala, Arg, Asn, Asp, Glu, Gln, Gly, His, Ile, Leu, Lys, Phe, Pro, Ser, Thr, Trp, Tyr or Val;
X₁₂ is Ala, Arg, Asn, Asp, Glu, Gln, Gly, His, Ile, Leu, Lys, Phe, Pro, Ser, Thr, Trp, Tyr or Val;
X₁₃ is Ala, Arg, Asn, Asp, Glu, Gln, Gly, His, Ile, Leu, Lys, Phe, Pro, Ser, Thr, Trp, Tyr or Val;
X₁₄ is Ala, Arg, Asn, Asp, Glu, Gln, Gly, His, Ile, Leu, Lys, Phe, Pro, Ser, Thr, Trp, Tyr or Val; and
X₁₅ is Ala, Arg, Asn, Asp, Glu, Gln, Gly, His, Ile, Leu, Lys, Phe, Pro, Ser, Thr, Trp, Tyr or Val.

TABLE 2
Residue distribution in the TCL2 library

| Residue | WT | |
|---|---|---|
| Position* | residues | Distribution in the TCL2 library |
| 22 | T | designed distribution |
| 23 | A | designed distribution |
| 24 | P | 50% P + designed distribution |
| 25 | D | designed distribution |
| 26 | A | 20% A + 20% G + designed distribution |
| 27 | A | designed distribution |
| 28 | F | 20% F, 20% I, 20% L, 20% V, 20% Y |
| 29 | D | 33% D, 33% E, 33% T |
| 75 | K | designed distribution |
| 76 | G | designed distribution |
| 77 | G | designed distribution |
| 78 | H | designed distribution |
| 79 | R | designed distribution |
| 80 | S | 100% S |
| 81 | N | designed distribution |
| 82 | P | 50% P + designed distribution |

*residue numbering is based on Tencon sequence of SEQ ID NO: 1

Subsequently, these libraries were improved by various ways, including building of the libraries on a stabilized Tencon framework (U.S. Pat. No. 8,569,227) that incorporates substitutions E11R/L17A/N46V/E861 (Tencon27; SEQ ID NO: 4) when compared to the wild type tencon as well as altering of the positions randomized in the BC and FG loops. Tencon27 is described in Int. Pat. Appl. No. WO2013049275. From this, new libraries designed to randomize only the FG loop of Tencon (library TCL9), or a combination of the BC and FG loops (library TCL7) were generated. These libraries were constructed for use with the cis-display system (Odegrip et al., Proc. Natl. Acad. Sci. USA 101: 2806-2810, 2004). The details of this design are shown below:

Stabilized Tencon (Tencon27)
(SEQ ID NO: 4)
LPAPKNLVVSRVTEDSARLSWTAPDAAFDSFLIQYQESEKVGEAIVL
TVPGSERSYDLTGLKPGTEYTVSIYGVKGGHRSNPLSAIFTT
TCL7 (randomized FG and BC loops)
(SEQ ID NO: 5)
LPAPKNLVVSRVTEDSARLSWX₁X₂X₃X₄X₅X₆X₇X₈X₉FDSFLIQY
QESEKVGEAIVLTVPGSERSYDLTGLKPGTEYTVSIYGVX₁₀X₁₁X₁₂
X₁₃X₁₄X₁₅X₁₆X₁₇X₁₈X₁₉SNPLSAIFTT;

wherein
X₁, X₂, X₃, X₄, X₅, X₆, X₁₀, X₁₁, X₁₂, X₁₃, X₁₄, X₁₅ and X₁₆ is A, D, E, F, G, H, I, K, L, N, P, Q, R, S, T, V, W or Y; and
X₇, X₈, X₉, X₁₇, X₁₈ and X₁₉, is A, D, E, F, G, H, I, K, L, N, P, Q, R, S, T, V, W, Y or deleted.

TCL9 (randomized FG loop)
(SEQ ID NO: 6)
LPAPKNLVVSRVTEDSARLSWTAPDAAFDSFLIQYQESEKVGEAIVL
TVPGSERSYDLTGLKPGTEYTVSIYGV X₁X₂X₃X₄X₅X₆X₇X₈X₉
X₁₀X₁₁X₁₂SNPLSAIFTT;
X₁, X₂, X₃, X₄, X₅, X₆ and X₇, is A, D, E, F,
G, H, I, K, L, N, P, Q, R, S, T, V, W or Y;
and X₈, X₉, X₁₀, X₁₁ and X₁₂ is A, D, E, F, G,
H, I, K, L, N, P, Q, R, S, T, V, W, Y
or deleted.

For library construction, DNA fragments encoding randomized BC loops (lengths 6-9 positions) or FG loops (lengths 7-12 positions) were synthesized using Slonomics technology (Sloning Biotechnology GmbH) so as to control the amino acid distribution of the library and to eliminate stop codons. Two different sets of DNA molecules randomizing either the BC loop or the FG loops were synthesized independently and later combined using PCR to produce the full library product.

### Construction of FG Loop Libraries (TCL9)

A set of synthetic DNA molecules consisting of a 5′ Tac promoter followed by the complete gene sequence of Tencon with the exception of randomized codons in the FG loop was produced (SEQ ID NOs: 26-31) For FG loop randomization, all amino acids except cysteine and methionine were encoded at equal percentages. The lengths of the diversified portion are such that they encode for 7, 8, 9, 10, 11, or 12 amino acids in the FG loop. Sub-libraries of each length variation were synthesized individually at a scale of 2 ug and then amplified by PCR using oligos Sloning-FOR (SEQ ID NO: 9) and Sloning-Rev (SEQ ID NO: 10).

The 3′ fragment of the library is a constant DNA sequence containing elements for display, including a PspOMI restriction site, the coding region of the repA gene, and the cis- and on elements. PCR reactions were performed to amplify this fragment using a plasmid (pCR4Blunt) (Invitrogen) as a template with M13 Forward and M13 Reverse primers. The resulting PCR products were digested by PspOMI overnight and gel-purified. To ligate the 5′ portion of library DNA to the 3′ DNA containing repA gene, 2 pmol (˜540 ng to 560 ng) of 5′ DNA was ligated to an equal molar (˜1.25 μg) of 3′ repA DNA in the presence of NotI and PspOMI enzyme and T4 ligase at 37° C. overnight. The ligated library product was amplified by using 12 cycles of PCR with oligos POP2250 (SEQ ID NO: 11) and DigLigRev (SEQ ID NO: 12). For each sub-library, the resulting DNA from 12 PCR reactions were combined and purified by Qiagen spin column. The yield for each sub-library of TCL9 ranged from 32-34 μg.

### Construction of FG/BC Loop Libraries (TCL7)

The TCL7 library provides for a library with randomized Tencon BC and FG loops. In this library, BC loops of lengths 6-9 amino acids were mixed combinatorically with randomized FG loops of 7-12 amino acids in length. Synthetic Tencon fragments BC6, BC7, BC8, and BC9 (SEQ ID NOs: 13-16, respectively) were produced to include the Tencon gene encoding for the N-terminal portion of the protein up to and including residue VX such that the BC loop is replaced with either 6, 7, 8, or 9 randomized amino acids, which are represented by the string of “N” in the sequences provided for herein. These fragments were synthesized prior to the discovery of L17A, N46V and E831 mutations (CEN5243) but these mutations were introduced in the molecular biology steps described below. In order to combine this fragment with fragments encoding for randomized FG loops, the following steps were taken.

First, a DNA fragment encoding the Tac promoter and the 5′ sequence of Tencon up to the nucleotide encoding for amino acid A17 (130mer-L17A, SEQ ID NO: 17) was produced by PCR using oligos POP2222ext (SEQ ID NO: 18) and LS1114 (SEQ ID NO: 19). This was done to include the L17A mutation in the library (CEN5243). Next, DNA fragments encoding for Tencon residues R18-V75 including randomized BC loops were amplified by PCR using BC6, BC7, BC8, or BC9 as a templates and oligos LS1115 (SEQ ID NO: 20) and LS1117 (SEQ ID NO: 21). This PCR step introduced a BsaI site at the 3′ end. These DNA fragments were subsequently joined by overlapping PCR using oligos POP2222ext and LS1117 as primers. The resulting PCR product of 240 bp was pooled and purified by Qiagen PCR purification kit. The purified DNA was digested with BsaI-HF and gel purified.

Fragments encoding the FG loop were amplified by PCR using FG7, FG8, FG9, FG10, FG11, and FG12 as templates with oligonucleotides SDG10 (SEQ ID NO: 22) and SDG24 (SEQ ID NO: 23) to incorporate a BsaI restriction site and N46V and E861 variations (CEN5243).

The digested BC fragments and FG fragments were ligated together in a single step using a 3-way ligation. Four ligation reactions in the 16 possible combinations were set up, with each ligation reaction combining two BC loop lengths with 2 FG loop lengths. Each ligation contained ˜300 ng of total BC fragment and 300 ng of the FG fragment. These 4 ligation pools were then amplified by PCR using oligos POP2222 (SEQ ID NO: 24) and SDG28 SEQ ID NO: 25). 7.5 μg of each reaction product were then digested with NotI and cleaned up with a Qiagen PCR purification column. 5.2 μg of this DNA, was ligated to an equal molar amount of RepA DNA fragment (˜14 μg) digested with PspOMI and the product amplified by PCR using oligos POP2222.

### Example 2: Generation of Tencon Libraries Having Alternative Binding Surfaces

The choice of residues to be randomized in a particular library design governs the overall shape of the interaction surface created. X-ray crystallographic analysis of an FN3 domain containing scaffold protein selected to bind maltose binding protein (MBP) from a library in which the BC, DE, and FG loops were randomized was shown to have a largely curved interface that fits into the active site of MBP (Koide et al., Proc. Natl. Acad. Sci. USA 104: 6632-6637, 2007). In contrast, an ankyrin repeat scaffold protein that was selected to bind to MBP was found to have a much more planar interaction surface and to bind to the outer surface of MBP distant from the active (Binz et al., Nat. Biotechnol. 22: 575-582, 2004). These results suggest that the shape of the binding surface of a scaffold molecule (curved vs. flat) may dictate what target proteins or specific epitopes on those target proteins are able to be bound effectively by the scaffold. Published efforts around engineering protein scaffolds containing FN3 domains for protein binding has relied on engineering adjacent loops for target binding, thus producing curved binding surfaces. This approach may limit the number of targets and epitopes accessible by such scaffolds.

Tencon and other FN3 domains contain two sets of CDR-like loops lying on the opposite faces of the molecule, the first set formed by the BC, DE, and FG loops, and the second set formed by the AB, CD, and EF loops. The two sets of loops are separated by the beta-strands that form the center of the FN3 structure. If the image of the Tencon is rotated by 90 degrees, an alternative surface can be visualized. This slightly concave surface is formed by the CD and FG loops and two antiparallel beta-strands, the C and the F beta-strands, and is herein called the C-CD-F-FG surface. The C-CD-F-FG surface can be used as a template to design libraries of protein scaffold interaction surfaces by randomizing a subset of residues that form the surface. Beta-strands have a repeating structure with the side chain of every other residue exposed to the surface of the protein. Thus, a library can be made by randomizing some or all surface exposed residues in the beta strands. By choosing the appropriate residues in the beta-strands, the inherent stability of the Tencon scaffold should be minimally compromised while providing a unique scaffold surface for interaction with other proteins.

Library TCL14 (SEQ ID NO: 7), was designed into Tencon27 scaffold (SEQ ID NO: 4).

A full description of the methods used to construct this library is described in US. Pat. Publ. No. 2013/0226834.

TCL14 library
(SEQ ID NO: 7):
LPAPKNLVVSRVTEDSARLSWTAPDAAFDSFX₁IX₂YX₃EX₄X₅X₆X₇
GEAIVLTVPGSERSYDLTGLKPGTEYX₈VX₉IX₁₀GVKGGX₁₁X₁₂
SX₁₃PLSAIFTT;

wherein
X₁, X₂, X₃, X₄, X₅, X₆, X₇, X₈, X₉, X₁₀, X₁₁, X₁₂ and X₁₃ are A, D, E, F, G, H, I, K, L, N, P, Q, R, S, T, V, W, Y, C or M.

The two beta strands forming the C-CD-F-FG surface in Tencon27 have a total of 9 surface exposed residues that could be randomized; C-strand: S30, L32, Q34, Q36; F-strand: E66, T68, 570, Y72, and V74, while the CD loop has 6 potential residues: S38, E39, K40, V41, G42, and E43 and the FG loop has 7 potential residues: K75, G76, G77, H78, R79, 580, and N81. Select residues were chosen for inclusion in the TCL14 design due to the larger theoretical size of the library if all 22 residues were randomized.

Thirteen positions in Tencon were chosen for randomizing: L32, Q34 and Q36 in C-strand, S38, E39, K40 and V41 in CD-loop, T68, S70 and Y72 in F-strand, H78, R79, and N81 in FG-loop. In the C and F strands S30 and E66 were not randomized as they lie just beyond the CD and FG loops and do not appear to be as apparently a part of the C-CD-F-FG surface. For the CD loop, G42 and E43 were not randomized as glycine, providing flexibility, can be valuable in loop regions, and E43 lies at the junction of the surface. The FG loop had K75, G76, G77, and S80 excluded. The glycines were excluded for the reasons above while careful inspection of the crystal structures revealed S80 making key contacts with the core to help form the stable FG loop. K75 faces away from the surface of the C-CD-F-FG surface and was a less appealing candidate for randomization. Although the above mentioned residues were not randomized in the original TCL14 design, they could be included in subsequent library designs to provide additional diversity for de novo selection or for example for an affinity maturation library on a select TCL14 target specific hit.

Subsequent to the production of TCL14, 3 additional Tencon libraries of similar design were produced. These two libraries, TCL19, TCL21 and TCL23, are randomized at the same positions as TCL14 (see above) however the distribution of amino acids occurring at these positions is altered (Table 3). TCL19 and TCL21 were designed to include an equal distribution of 18 natural amino acids at every position (5.55% of each), excluding only cysteine and methionine. TCL23 was designed such that each randomized position approximates the amino acid distribution found in the HCDR3 loops of functional antibodies (Birtalan et al., J. Mol. Biol. 377: 1518-1528, 2008) as described in Table 3. As with the TCL21 library, cysteine and methionine were excluded.

A third additional library was built to expand potential target binding surface of the other libraries library. In this library, TCL24, 4 additional Tencon positions were randomized as compared to libraries TCL14, TCL19, TCL21, and TCL23. These positions include N46 and T48 from the D strand and S84 and 186 from the G strand. Positions 46, 48, 84, and 86 were chosen in particular as the side chains of these residues are surface exposed from beta-strands D and G and lie structurally adjacent to the randomized portions of the C and F strand, thus increasing the surface area accessible for binding to target proteins. The amino acid distribution used at each position for TCL24 is identical to that described for TCL19 and TCL21 in Table 3.

TCL24 Library
(SEQ ID NO: 8)
LPAPKNLVVSRVTEDSARLSWTAPDAAFDSFX₁IX₂YX₃EX₄X₅X₆X₇
GEAIX₈LX₉VPGSERSYDLTGLKPGTEYX₁₀VX₁₁IX₁₂GVKGGX₁₃
X₁₄SX₁₅PLX₁₆AX₁₇FTT;

wherein
X₁, X₂, X₃, X₄, X₅, X₆, X₁₀, X₁₁, X₁₂, X₁₃, X₁₄, X₁₅, X₁₆ and X₁₇ are A, D, E, F, G, H, I, K, L, N, P, Q, R, S, T, V, Y or W.

TABLE 3
Amino acid frequency (%) at each randomized position for
TCL21, TCL23, and TCL24.

| Amino Acid | TCL19 | TCL21 | TCL23 | TCL24 |
|---|---|---|---|---|
| Ala | 5.6 | 5.6 | 6.0 | 5.6 |
| Arg | 5.6 | 5.6 | 6.0 | 5.6 |
| Asn | 5.6 | 5.6 | 3.9 | 5.6 |
| Asp | 5.6 | 5.6 | 7.5 | 5.6 |
| Cys | 0.0 | 0.0 | 0.0 | 0.0 |
| Gln | 5.6 | 5.6 | 1.5 | 5.6 |
| Glu | 5.6 | 5.6 | 2.5 | 5.6 |
| Gly | 5.6 | 5.6 | 15.0 | 5.6 |
| His | 5.6 | 5.6 | 2.3 | 5.6 |
| Ile | 5.6 | 5.6 | 2.5 | 5.6 |
| Leu | 5.6 | 5.6 | 5.0 | 5.6 |
| Lys | 5.6 | 5.6 | 1.5 | 5.6 |
| Met | 0.0 | 0.0 | 0.0 | 0.0 |
| Phe | 5.6 | 5.6 | 2.5 | 5.6 |
| Pro | 5.6 | 5.6 | 4.0 | 5.6 |
| Ser | 5.6 | 5.6 | 10.0 | 5.6 |
| Thr | 5.6 | 5.6 | 4.5 | 5.6 |
| Trp | 5.6 | 5.6 | 4.0 | 5.6 |
| Tyr | 5.6 | 5.6 | 17.3 | 5.6 |
| Val | 5.6 | 5.6 | 4.0 | 5.6 |

### Generation of TCL21, TCL23, and TCL24 Libraries

The TCL21 library was generated using Colibra library technology (Isogenica) in order to control amino acid distributions. TCL19, TCL23, and TCL24 gene fragments were generated using Slonomics technology (Morphosys) to control amino acid distributions. PCR was used to amplify each library following initial synthesis followed by ligation to the gene for RepA in order to be used in selections using the CIS-display system (Odegrip et al., Proc. Natl. Acad. Sci. USA 101: 2806-2810, 2004) as described above for the loop libraries.

### Example 3: Selection of Fibronectin Type III (FN3) Domains that Bind CD71 Panning and Biochemical Screening

FN3 domains specific for human CD71 were selected via CIS-Display (Odegrip et al 2004) using recombinant biotinylated CD71 extracellular domain (Sino Biologics) with an N-terminal 6His tag. For in vitro transcription and translation (ITT), 3 μg of DNA from FN3 domain libraries TCL18, TCL19, TCL21, TCL23, and TCL24 were used, with unbound library members removed by washing. DNA was eluted from the target protein by heating and amplified by PCR using KOD polymerase for further rounds of panning. High affinity binders were isolated by successively lowering the concentration of target CD71 during each round from 400 nM to 100 nM and increasing the washing stringency. Outputs from the fifth round panning were subjected to four additional rounds of off-rate selection. The biotinylated target antigen concentration was reduced from 25 nM in rounds 6 and 7 to 2.5 nM in rounds 8 and 9.

Following panning, genes encoding the selected FN3 domains were amplified by PCR, subcloned into a pET vector modified to include a ligase independent cloning site, and transformed into BL21 (DE3) (Stratagene) cells for soluble expression in E. coli using standard molecular biology techniques. A gene sequence encoding a C-terminal poly-histidine tag was added to each FN3 domain to enable purification and detection.

To screen for FN3 domains that specifically bind CD71, streptavidin-coated Maxisorp plates (Nunc catalog 436110) were blocked for 1 hour in Starting Block T20 (Pierce) and then coated with biotinylated CD71 (using same antigen as in panning) or negative controls (an unrelated Fc-fused recombinant protein and human serum albumin) for 1 hour. Plates were rinsed with TBST and diluted lysate was applied to plates for 1 hour. Following additional rinses, wells were treated with HRP-conjugated anti-V5 tag antibody (Abcam, ab1325), for 1 hour and then assayed with POD (Roche, 11582950001). The DNA from FN3 domain lysates with signals at least 10-fold ELISA signal above that of streptavidin controls were sequenced resulting in 23 unique, readable FN3 domain sequences isolated from Round 9 screening (Table 4).

TABLE 4
Summary of Screening Hits

| | | | SEQ |
|---|---|---|---|
| hCD71 | HSA | huCD71/Fc | ID |
| 517840 | 11120 | 46.6 | 33 |
| 310480 | 25920 | 12.0 | 34 |
| 3244640 | 1520 | 2134.6 | 35 |
| 3297120 | 6160 | 535.2 | 36 |
| 1271360 | 2720 | 467.4 | 37 |
| 840480 | 4160 | 202.0 | 38 |
| 506800 | 4160 | 121.8 | 39 |
| 220240 | 2960 | 74.4 | 40 |
| 4267840 | 10080 | 423.4 | 41 |
| 2827520 | 5920 | 477.6 | 42 |
| 1621680 | 8160 | 198.7 | 43 |
| 175760 | 3920 | 44.8 | 44 |
| 1926160 | 2880 | 668.8 | 45 |
| 112560 | 3040 | 37.0 | 46 |
| 264800 | 5200 | 50.9 | 47 |
| 943120 | 2800 | 336.8 | 48 |
| 10915200 | 11520 | 947.5 | 49 |
| 10786240 | 2400 | 4494.3 | 50 |
| 9709680 | 4240 | 2290.0 | 51 |
| 10112800 | 1760 | 5745.9 | 52 |
| 1007840 | 9120 | 110.5 | 53 |
| 6987520 | 6160 | 1134.3 | 54 |
| 11142160 | 7760 | 1435.8 | 55 |
| 11339360 | 7520 | 1507.9 | 56 |
| 1903600 | 16880 | 112.8 | 57 |
| 301680 | 4800 | 62.9 | 58 |
| 1946880 | 3200 | 608.4 | 59 |
| 4479040 | 6480 | 691.21 | 60 |
| 4900320 | 10640 | 460.56 | 61 |

### Size Exclusion Chromatography Analysis

Size exclusion chromatography was used to determine the aggregation state of anti-CD71 FN3 domains. Aliquots (10 μL) of each purified FN3 domain were injected onto a Superdex 75 5/150 column (GE Healthcare) at a flow rate of 0.3 mL/min in a mobile phase of PBS pH 7.4. Elution from the column was monitored by absorbance at 280 nm. Tencon protein was included in each run as a control. Agilent ChemStation software was used to analyze the elution profiles. Selected SEC parameters for the 18 identified FN3 domains are listed in Table 5.

TABLE 5
Summary of Size Exclusion Chromatography Analysis

| SEQ | RT | Height | |
|---|---|---|---|
| ID | (min) | (mAU) | Y/N |
| 33 | 5.616 | 21578 | Y |
| 34 | 5.729 | 19210 | Y |
| 35 | 5.818 | 36983 | Y |
| 36 | 6.008 | 59654 | Y |
| 37 | 5.486 | 33495 | Y |
| 38 | 5.608 | 32759 | Y |
| 39 | 6.508 | 40533 | Y |
| 40 | 6.043 | 42995 | Y |
| 41 | 6.535 | 12055 | N |
| 42 | 6.243 | 114847 | Y |
| 43 | 6.736 | 64318 | Y |
| 44 | 6.389 | 33849 | Y |
| 45 | 6.196 | 16535 | Y |
| 46 | 5.962 | 56696 | Y |
| 47 | 6.799 | 61095 | Y |
| 48 | 5.405 | 24438 | Y |
| 49 | 6.149 | 118941 | Y |
| 50 | 6.496 | 122793 | Y |
| 51 | 7.729 | 17618 | N |
| 52 | 6.316 | 87040 | Y |
| 53 | 6.118 | 87022 | Y |
| 54 | 5.972 | 34366 | Y |
| 55 | 6.06 | 35099 | Y |
| 56 | 5.496 | 28177 | Y |
| 57 | 6.175 | 13973 | Y |
| 58 | 5.862 | 45603 | Y |
| 59 | 5.589 | 85517 | Y |
| 60 | 5.671 | 8.6 | Y |
| 61 | 5.752 | 8.9 | Y |

### High-Throughput Expression and Conjugation

Clones identified were grown in duplicate 5 mL cultures in 24 well deep block plates. Briefly, 5 mL/well of TB media supplemented with 50 μg/mL Kanamycin was seeded with 150 μL of overnight culture and grown for about 3 hours at 37° C. with shaking at 220 rpm (OD600˜1). Cultures were induced with IPTG to a final concentration of 1 mM for an additional 4 hours at 37° C., 220 rpm. Bacterial pellets were recovered by centrifugation at 2250×g for 15 minutes. 600 μL/well BugBuster HT (Novagen) supplemented with lysozyme (Sigma) at 0.2 mg/mL was added to each well; pellets were dissociated by pipette and then shaken vigorously on a platform shake for about 30 minutes until pellets were lysed. Plates were spun at 2250×g for 15 minutes to clarify lysates and the 2 600-μL aliquots for each sample were combined. His-tagged FN3 domains were purified on His Trap plates (GE) according to the manufacturer's instructions followed by buffer exchange into TBS using Zeba Spin 7K desalt plates (Thermo Scientific). Protein concentrations were assessed by Nanodrop. For conjugation to GlyGly-VC-MMAF, FN3 domain (30 μM) was mixed with 150 μM GlyGlyVC-MMAF (Concortis) and 1 μM Sortase A in a total volume of 200 μL. Conjugations were allowed to proceed for 1.5 hours at room temperature and purified again using a 96 well His Multitrap HP plate from GE Healthcare according to the manufacturer's instructions. Buffer exchange into PBS was achieved using Zeba desalt plates followed by sterile filtering using Multiscreen HTS GV plates (Durapore) with centrifugation at 3000×g for 2 mins. Concentrations were assessed by Nanodrop.

### Identification of SK-BR3 Binding FN3 Domains

SK-BR-3 cells are cultured in McCoy's 5a Medium+10% Fetal Bovine Serum. FN3 dilutions are prepared in FACS buffer. 50,000 SK-BR-3 cells are added to each well; media was aspirated after centrifugation and cells are resuspended in 100 μL of FACS buffer containing HiLyte labeled FN3 domains. Cells are incubated for 2 hours at 37° C., 5% CO2. Cells are rinsed 3× with FACS buffer and finally resuspended in 100 μL of FACS buffer. Fluorescence is detected by Intellictye. Cell populations are identified by the FSC-SSC dot plot followed by recording of the FL4 MFI. Data are normalized to the average of 8 unstained cells and dose response curves are fit using GraphPad.

### Binding of Selected Clones by Dose-Response ELISA

Selected clones are analyzed by ELISA to determine EC50 values for binding. Briefly, Maxisorb plates are coated with streptavidin at 5 μg/ml overnight at 4 C. Plates were then blocked with StartingBlock (ThermoFisher) at room temperature for 1 hour and then washed with TBS-Tween. Biotinylated CD71 (2 μg/ml) was captured onto the streptavidin plates and serially diluted Centyrins were added to appropriate wells for 1 hour at room temperature. After washing, bound Centyrin was detected with anti-V5 tag antibody, which is conjugated to HRP and POD substrate and a luminescence plate reader. Luminescence values are plotted as a function of concentration and fit to a dose response using PRISM to determine EC50 values for binding.

Identification of internalizing FN3 domains via toxin conjugates. The FN3 domains were conjugated to the cytotoxic tubulin inhibitor momomethyl auristatin F (MMAF) via an enzyme-cleavable Val-Cit linker or a non-cleavable PEG4 linker (VC-MMAF) using the methodology described for the NEM conjugation. Cell killing was assessed by measuring viability of the SKBR-3 cells following exposure to the cysteine variant-cytotoxin conjugates. Cells are plated in white-well, opaque bottomed, tissue culture-treated plates (Fisher, P115042) at 3000/well in 50 μL/well of phenol red RPMI media (Gibco, 11875093) with 10% fetal bovine serum (Gibco). Cells are allowed to attach overnight at 37° C. in a humidified 5% CO2 atmosphere. Cells are treated with 25 uL of fresh media and 25 uL of 4× inhibitor made up in fresh media. Cell viability is determined by an endpoint assay with Cell TiterGlo (Promega) at 72 hours. IC50 values are determined by fitting data to the equation for a sigmoidal dose response with variable slope using GraphPad Prism (GraphPad Software). The results are illustrated in Table 6 and demonstrate that the FN3 domains that bind to CD71 were internalized and cytotoxic.

TABLE 6
IC50 of CD71 FN3- MMAF conjugate molecules in SKBR-3 Cells

| | IC50 |
|---|---|
| SEQ ID | (nM) |
| 33 | 3.27 |
| 34 | 0.37 |
| 35 | 2.5 |
| 36 | 7.1 |
| 37 | 0.15 |
| 38 | 3.82 |
| 39 | 0.52 |
| 40 | 3 |
| 41 | 4.7 |
| 42 | 0.19 |
| 43 | 0.069 |
| 44 | 2.5 |
| 45 | 8.3 |
| 46 | 2.69 |
| 47 | 5.9 |
| 48 | 0.42 |
| 49 | 3 |
| 50 | 3.1 |
| 51 | 4.9 |
| 52 | 6.3 |
| 53 | 0.07 |
| 54 | 0.4 |
| 55 | 0.026 |
| 56 | 0.24 |
| 57 | 3.13 |
| 58 | 7.7 |
| 59 | 4 |
| 60 | 0.45 |
| 61 | 1.93 |

### Bivalent FN3 Protein

A bivalent FN3 protein is produced using two FN3 domains connected by a 4 repeat G/S linker. The bivalent FN3 protein is conjugated to VC-MMAF as described and assessed for cytotoxicity in SK-BR3 cells. The IC50 value for bivalent molecule is found to be better.

### Competition for Transferrin Binding and Internalization

FN3 domain VCMMAF conjugates were screened for competition with human transferrin using the cytotoxicity assay described above. FN3 domains were screened in the absence or presence of 0.6 uM holo-human transferrin (T0665-100MG). The IC50 values for FN3 domain toxin conjugates on SK-BR3 cells screened in the absence or presence of competitor are shown in Table 7.

TABLE 7
IC50 of CD71 FN3 domain - MMAF conjugate molecules on
SKBR-3 cells +/− human transferrin

| | IC50 (nM) | |
|---|---|---|
| | huTf | no |
| SEQ ID | competitor | competitor |
| 33 | ~200 | 4.4 |
| 34 | 195.9 | 1.3 |
| 35 | 41.5 | 0.7 |
| 37 | 142.8 | 0.7 |
| 38 | ~120 | 4 |
| 39 | 31.1 | 1.2 |
| 40 | ~100 | 2.2 |
| 41 | 0.5 | 0.01 |
| 42 | ~70 | 2.4 |
| 43 | ~80 | 0.97 |
| 45 | 0.9 | 0.05 |
| 46 | ~100 | 3 |
| 47 | ~85 | 1.6 |
| 48 | ~70 | 1.3 |
| 53 | ~90 | 3.6 |
| 54 | 5 | 0.13 |
| 55 | 5.5 | 0.15 |
| 56 | 14.7 | 0.35 |
| 57 | 5.3 | 0.38 |
| 60 | 14.4 | 0.21 |
| 62 | 0.60 | >0.005 |

SEQ ID NO: 1 = Original Tencon Sequence
LPAPKNLVVSEVTEDSLRLSWTAPDAAFDSFLIQYQESEKVGEAIN
LTVPGSERSYDLTGLKPGTEYTVSIYGVKGGHRSNPLSAEFTT
SEQ ID NO: 2 = TCL1 library
LPAPKNLVVSEVTEDSLRLSWTAPDAAFDSFLIQYQESEKVGEAINL
TVPGSERSYDLTGLKPGTEYTVSIYGV(X)₇₋₁₂PLSAEFTT;

wherein
X₁, X₂, X₃, X₄, X₅, X₆, X₇ is any amino acid; and
X₈, X₉, X₁₀, X₁₁ and X₁₂ are any amino acid or deleted

SEQ ID NO: 3 = TCL2 library
LPAPKNLVVSEVTEDSLRLSWX₁X₂X₃X₄X₅X₆X₇X₈SFLIQYQESEK
VGEAINLTVPGSERSYDLTGLKPGTEYTVSIYGVX₉X₁₀X₁₁X₁₂X₁₃
SX₁₄X₁₅LSAEFTT;

wherein
X₁ is Ala, Arg, Asn, Asp, Glu, Gln, Gly, His, Ile, Leu, Lys, Phe, Pro, Ser, Thr, Trp, Tyr or Val;
X₂ is Ala, Arg, Asn, Asp, Glu, Gln, Gly, His, Ile, Leu, Lys, Phe, Pro, Ser, Thr, Trp, Tyr or Val;
X₃ Ala, Arg, Asn, Asp, Glu, Gln, Gly, His, Ile, Leu, Lys, Phe, Pro, Ser, Thr, Trp, Tyr or Val;
X₄ is Ala, Arg, Asn, Asp, Glu, Gln, Gly, His, Ile, Leu, Lys, Phe, Pro, Ser, Thr, Trp, Tyr or Val;
X₅ is Ala, Arg, Asn, Asp, Glu, Gln, Gly, His, Ile, Leu, Lys, Phe, Pro, Ser, Thr, Trp, Tyr or Val;
X₆ is Ala, Arg, Asn, Asp, Glu, Gln, Gly, His, Ile, Leu, Lys, Phe, Pro, Ser, Thr, Trp, Tyr or Val;
X₇ is Phe, Ile, Leu, Val or Tyr;
X₈ is Asp, Glu or Thr;
X₉ is Ala, Arg, Asn, Asp, Glu, Gln, Gly, His, Ile, Leu, Lys, Phe, Pro, Ser, Thr, Trp, Tyr or Val;
X₁₀ is Ala, Arg, Asn, Asp, Glu, Gln, Gly, His, Ile, Leu, Lys, Phe, Pro, Ser, Thr, Trp, Tyr or Val;
X₁₁ is Ala, Arg, Asn, Asp, Glu, Gln, Gly, His, Ile, Leu, Lys, Phe, Pro, Ser, Thr, Trp, Tyr or Val;
X₁₂ is Ala, Arg, Asn, Asp, Glu, Gln, Gly, His, Ile, Leu, Lys, Phe, Pro, Ser, Thr, Trp, Tyr or Val;
X₁₃ is Ala, Arg, Asn, Asp, Glu, Gln, Gly, His, Ile, Leu, Lys, Phe, Pro, Ser, Thr, Trp, Tyr or Val;
X₁₄ is Ala, Arg, Asn, Asp, Glu, Gln, Gly, His, Ile, Leu, Lys, Phe, Pro, Ser, Thr, Trp, Tyr or Val; and
X₁₅ is Ala, Arg, Asn, Asp, Glu, Gln, Gly, His, Ile, Leu, Lys, Phe, Pro, Ser, Thr, Trp, Tyr or Val.

SEQ ID NO: 4 = Stabilized Tencon
LPAPKNLVVSRVTEDSARLSWTAPDAAFDSFLIQYQESEKVGEAIV
LTVPGSERSYDLTGLKPGTEYTVSIYGVKGGHRSNPLSAIFTT
SEQ ID NO: 5 = TCL7 (FG and BC loops)
LPAPKNLVVSRVTEDSARLSWX₁X₂X₃X₄X₅X₆X₇X₈X₉FDSFLIQYQE
SEKVGEAIVLTVPGSERSYDLTGLKPGTEYTVSIYGVX₁₀X₁₁X₁₂X₁₃
X₁₄X₁₅X₁₆X₁₇X₁₈X₁₉SNPLSAIFTT;

wherein
X₁, X₂, X₃, X₄, X₅, X₆, X₁₀, X₁₁, X₁₂, X₁₃, X₁₄, X₁₅ and X₁₆ are A, D, E, F, G, H, I, K, L, N, P, Q, R, S, T, V, W or Y; and
X₇, X₈, X₉, X₁₇, X₁₈ and X₁₉, are A, D, E, F, G, H, I, K, L, N, P, Q, R, S, T, V, W, Y or deleted

SEQ ID NO: 6 = TCL9 (FG loop)
LPAPKNLVVSRVTEDSARLSWTAPDAAFDSFLIQYQESEKVGEAIVL
TVPGSERSYDLTGLKPGTEYTVSIYGVX₁X₂X₃X₄X₅X₆X₇X₈X₉X₁₀X₁₁
X₁₂SNPLSAIFTT;

wherein
X₁, X₂, X₃, X₄, X₅, X₆ and X₇, is A, D, E, F, G, H, I, K, L, N, P, Q, R, S, T, V, W or Y; and
X₈, X₉, X₁₀, X₁₁ and X₁₂ is A, D, E, F, G, H, I, K, L, N, P, Q, R, S, T, V, W, Y or deleted.

TCL14 library
(SEQ ID NO: 7)
LPAPKNLVVSRVTEDSARLSWTAPDAAFDSFX₁IX₂YX₃EX₄X₅X₆X₇
GEAIVLTVPGSERSYDLTGLKPGTEYX₈VX₉IX₁₀GVKGGX₁₁X₁₂S
X₁₃PLSAIFTT;

wherein
X₁, X₂, X₃, X₄, X₅, X₆, X₇, X₈, X₉, X₁₀, X₁₁, X₁₂ and X₁₃ are A, D, E, F, G, H, I, K, L, N, P, Q, R, S, T, V, W, Y, C or M.

TCL24 Library
(SEQ ID NO: 8)
LPAPKNLVVSRVTEDSARLSWTAPDAAFDSFX₁IX₂YX₃EX₄X₅X₆X₇
GEAIX₈LX₉VPGSERSYDLTGLKPGTEYX₁₀VX₁₁IX₁₂GVKGGX₁₃
X₁₄SX₁₅PLX₁₆AX₁₇FTT;

wherein
X₁, X₂, X₃, X₄, X₅, X₆, X₁₀, X₁₁, X₁₂, X₁₃, X₁₄, X₁₅, X₁₆ and X₁₇ are A, D, E, F, G, H, I, K, L, N, P, Q, R, S, T, V, Y or W.

```
SEQ ID NO: 9 = Sloning-FOR
GTGACACGGCGGTTAGAAC
SEQ ID NO: 10 = Sloning-REV
GCCTTTGGGAAGCTTCTAAG
SEQ ID NO: 11 = POP2250
CGGCGGTTAGAACGCGGCTACAATTAATAC
SEQ ID NO: 12 = DigLigRev
CATGATTACGCCAAGCTCAGAA
SEQ ID NO: 13 = BC9
GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTGAAGTTACCGAAGACTCTCT
GCGTCTGTCTTGGNNNNNNNNNNNNNNNNNNNNNNNNNNNTTYGAC
TCTTTCCTGATCCAGTACCAGGAATCTGAAAAAGTTGGTGAAGCGA
TCAACCTGACCGTTCCGGGTTCTGAACGTTCTTACGACCTGACCGG
TCTGAAACCGGGTACCGAATACACCGTTTCTATCTACGGTGTTCT
TAGAAGCTTCCCAAAGGC
(wherein N is any base)
SEQ ID NO: 14 = BC8
GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTGAAGTTACCGAAGACTCTCT
GCGTCTGTCTTGGNNNNNNNNNNNNNNNNNNNNNNNNTTYGACTCT
TTCCTGATCCAGTACCAGGAATCTGAAAAAGTTGGTGAAGCGATCA
ACCTGACCGTTCCGGGTTCTGAACGTTCTTACGACCTGACCGGTCT
GAAACCGGGTACCGAATACACCGTTTCTATCTACGGTGTTCTTAG
AAGCTTCCCAAAGGC
(wherein N is any base)
SEQ ID NO: 15 = BC7
GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTGAAGTTACCGAAGACTCTCT
GCGTCTGTCTTGGNNNNNNNNNNNNNNNNNNNNNTTYGACTCTTTC
CTGATCCAGTACCAGGAATCTGAAAAAGTTGGTGAAGCGATCAACC
TGACCGTTCCGGGTTCTGAACGTTCTTACGACCTGACCGGTCTGAA
ACCGGGTACCGAATACACCGTTTCTATCTACGGTGTTCTTAGAAGC
TTCCCAAAGGC
(wherein N is any base)
SEQ ID NO: 16 = BC6
GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTGAAGTTACCGAAGACTCTCT
GCGTCTGTCTTGGNNNNNNNNNNNNNNNNNNTTYGACTCTTTCCTG
ATCCAGTACCAGGAATCTGAAAAAGTTGGTGAAGCGATCAACCTGA
CCGTTCCGGGTTCTGAACGTTCTTACGACCTGACCGGTCTGAAACC
GGGTACCGAATACACCGTTTCTATCTACGGTGTTCTTAGAAGCTTC
CCAAAGGC
(wherein N is any base)
SEQ ID NO: 17 = 130mer-L17A
CGGCGGTTAGAACGCGGCTACAATTAATACATAACCCCATCCCCCT
GTTGACAATTAATCATCGGCTCGTATAATGTGTGGAATTGTGAGCG
GATAACAATTTCACACAGGAAACAGGATCTACCATGCTG
SEQ ID NO: 18 = POP222ext
CGG CGG TTA GAA CGC GGC TAC AAT TAA TAC
SEQ ID NO: 19 = LS1114
CCA AGA CAG ACG GGC AGA GTC TTC GGT AAC GCG
AGA AAC AAC CAG GTT TTT CGG CGC CGG CAG CAT
GGT AGA TCC TGT TTC
SEQ ID NO: 20 = LS1115
CCG AAG ACT CTG CCC GTC TGT CTT GG
SEQ ID NO: 21 = LS1117
CAG TGG TCT CAC GGA TTC CTG GTA CTG GAT CAG
GAA AGA GT GAA
SEQ ID NO: 22 = SDG10
CATGCGGTCTCTTCCGAAAAAGTTGGTGAAGCGATCGTCCTGACCG
TTCCGGGT
SEQ ID NO: 23 = SDG24
GGTGGTGAAGATCGCAGACAGCGGGTTAG
SEQ ID NO: 24 = POP2222
CGGCGGTTAGAACGCGGCTAC
SEQ ID NO: 25 = SDG28
AAGATCAGTTGCGGCCGCTAGACTAGAACCGCTGCCACCGCCGGTG
GTGAAGATCGCAGAC
SEQ ID NO: 26 = FG12
GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTCGCGTTACCGAAGACTCTGC
GCGTCTGTCTTGGACCGCGCCGGACGCGGCGTTCGACTCTTTCCTG
ATCCAGTACCAGGAATCTGAAAAAGTTGGTGAAGCGATCGTGCTGA
CCGTTCCGGGTTCTGAACGTTCTTACGACCTGACCGGTCTGAAACC
GGGTACCGAATACACCGTTTCTATCTACGGTGTTNNNNNNNNNNNN
NNNNNNNNNNNNNNNNNNNNNNNNTCTAACCCGCTGTCTGCGATCT
TCACCACCGGCGGTCACCATCACCATCACCATGGCAGCGGTTCTAG
TCTAGCGGCCGCAACTGATCTTGGC
(wherein N is any base)
SEQ ID NO: 27 = FG11
GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTCGCGTTACCGAAGACTCTGC
GCGTCTGTCTTGGACCGCGCCGGACGCGGCGTTCGACTCTTTCCTG
ATCCAGTACCAGGAATCTGAAAAAGTTGGTGAAGCGATCGTGCTGA
CCGTTCCGGGTTCTGAACGTTCTTACGACCTGACCGGTCTGAAACC
GGGTACCGAATACACCGTTTCTATCTACGGTGTTNNNNNNNNNNNN
NNNNNNNNNNNNNNNNNNNNNTCTAACCCGCTGTCTGCGATCTTCA
CCACCGGCGGTCACCATCACCATCACCATGGCAGCGGTTCTAGTCT
AGCGGCCGCAACTGATCTTGGC
(wherein N is any base)
SEQ ID NO: 28 = FG10
  GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTCGCGTTACCGAAGACTCTGC
GCGTCTGTCTTGGACCGCGCCGGACGCGGCGTTCGACTCTTTCCTG
ATCCAGTACCAGGAATCTGAAAAAGTTGGTGAAGCGATCGTGCTGA
CCGTTCCGGGTTCTGAACGTTCTTACGACCTGACCGGTCTGAAACC
GGGTACCGAATACACCGTTTCTATCTACGGTGTTNNNNNNNNNNNN
NNNNNNNNNNNNNNNNNNTCTAACCCGCTGTCTGCGATCTTCACCA
CCGGCGGTCACCATCACCATCACCATGGCAGCGGTTCTAGTCTAGC
GGCCGCAACTGATCTTGGC
(wherein N is any base)
SEQ ID NO: 29 = FG9
  GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTCGCGTTACCGAAGACTCTGC
GCGTCTGTCTTGGACCGCGCCGGACGCGGCGTTCGACTCTTTCCTG
ATCCAGTACCAGGAATCTGAAAAAGTTGGTGAAGCGATCGTGCTGA
CCGTTCCGGGTTCTGAACGTTCTTACGACCTGACCGGTCTGAAACC
GGGTACCGAATACACCGTTTCTATCTACGGTGTTNNNNNNNNNNNN
NNNNNNNNNNNNNNNTCTAACCCGCTGTCTGCGATCTTCACCACCG
GCGGTCACCATCACCATCACCATGGCAGCGGTTCTAGTCTAGCGGC
CGCAACTGATCTTGGC
(wherein N is any base)
SEQ ID NO: 30 = FG8
  GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTCGCGTTACCGAAGACTCTGC
GCGTCTGTCTTGGACCGCGCCGGACGCGGCGTTCGACTCTTTCCTG
ATCCAGTACCAGGAATCTGAAAAAGTTGGTGAAGCGATCGTGCTGA
CCGTTCCGGGTTCTGAACGTTCTTACGACCTGACCGGTCTGAAACC
GGGTACCGAATACACCGTTTCTATCTACGGTGTTNNNNNNNNNNNN
NNNNNNNNNNNNTCTAACCCGCTGTCTGCGATCTTCACCACCGGCG
GTCACCATCACCATCACCATGGCAGCGGTTCTAGTCTAGCGGCCGC
AACTGATCTTGGC
(wherein N is any base)
SEQ ID NO: 31 = FG7
  GTGACACGGCGGTTAGAACGCGGCTACAATTAATACATAACCCCAT
CCCCCTGTTGACAATTAATCATCGGCTCGTATAATGTGTGGAATTG
TGAGCGGATAACAATTTCACACAGGAAACAGGATCTACCATGCTGC
CGGCGCCGAAAAACCTGGTTGTTTCTCGCGTTACCGAAGACTCTGC
GCGTCTGTCTTGGACCGCGCCGGACGCGGCGTTCGACTCTTTCCTG
ATCCAGTACCAGGAATCTGAAAAAGTTGGTGAAGCGATCGTGCTGA
CCGTTCCGGGTTCTGAACGTTCTTACGACCTGACCGGTCTGAAACC
GGGTACCGAATACACCGTTTCTATCTACGGTGTTNNNNNNNNNNNN
NNNNNNNNNTCTAACCCGCTGTCTGCGATCTTCACCACCGGCGGTC
ACCATCACCATCACCATGGCAGCGGTTCTAGTCTAGCGGCCGCAACT
GATCTTGGC
(wherein N is any base)
SEQ ID NO: 32 = human mature CD71
MTKEYQDLQHLDNEESDHHQLRKGPPPPQPLLQRLCSGPRLLLLSL
GLSLLLLVVVCVIGSQNSQLQEELRGLRETFSNFTASTEAQVKGLS
TQGGNVGRKMKSLESQLEKQQKDLSEDHSSLLLHVKQFVSDLRSLS
CQMAALQGNGSERTCCPVNWVEHERSCYWFSRSGKAWADADNYCRL
EDAHLVVVTSWEEQKFVQHHIGPVNTWMGLHDQNGPWKWVDGTDYE
TGFKNWRPEQPDDWYGHGLGGGEDCAHFTDDGRWNDDVCQRPYRWV
CETELDKASQEPPLL
SEQ ID NO: 80 = human mature CD71
extracellular domain
QNSQLQEELRGLRETFSNFTASTEAQVKGLSTQGGNVGRKMKSLES
QLEKQQKDLSEDHSSLLLHVKQFVSDLRSLSCQMAALQGNGSERTC
CPVNWVEHERSCYWFSRSGKAWADADNYCRLEDAHLVVVTSWEEQK
FVQHHIGPVNTWMGLHDQNGPWKWVDGTDYETGFKNWRPEQPDDWY
GHGLGGGEDCAHFTDDGRWNDDVCQRPYRWVCETELDKASQEPPLL
```

| SEQ | Amino Acid sequence of FN3 domains that |
|---|---|
| ID | bind to CD71 |
| 33 | MLPAPKNLVVSRVTEDSARLSWTAPDAAFDSFTIQYEELTTVGEA |
| | IYLRVPGSERSYDLTGLKPGTEYVVWIEGVKGGLRSNPLGAAFTT |
| 34 | MLPAPKNLVVSRVTEDSARLSWTAPDAAFDSFAITYIEWWDVGEA |
| | IGLKVPGSERSYDLTGLKPGTEYRVHIQGVKGGNNSYPLDALFTT |
| 35 | MLPAPKNLVVSRVTEDSARLSWTAPDAAFDSFEIAYFEAIWNGEA |
| | IYLTVPGSERSYDLTGLKPGTEYQVEIRGVKGGPTSRPLFAWFTT |
| 36 | MLPAPKNLVVSRVTEDSARLSWTAPDAAFDSFTITYIEWWENGEA |
| | IALSVPGSERSYDLTGLKPGTEYQVGIAGVKGGYKSYPLWALFTT |
| 37 | MLPAPKNLVVSRVTEDSARLSWTAPDAAFDSFHIIYTEEEKEGEA |
| | IYLRVPGSERSYDLTGLKPGTEYLVEIEGVKGGKRSVPLNASFTT |
| 38 | MLPAPKNLVVSRVTEDSARLSWTAPDAAFDSFHIAYEESHTTGEA |
| | IFLRVPGSERSYDLTGLKPGTEYSVSIEGVKGGHYSPPLTAKFTT |
| 39 | MLPAPKNLVVSRVTEDSARLSWTAPDAAFDSFDIDYREWWTLGEA |
| | IVLTVPGSERSYDLTGLKPGTEYYVNIQGVKGGLRSYPLSAIFTT |
| 40 | MLPAPKNLVVSRVTEDSARLSWTAPDAAFDSFVIEYWEYVGHGEA |
| | IVLTVPGSERSYDLTGLKPGTEYSVGIYGVKGGSLSRPLSAIFTT |
| 41 | MLPAPKNLVISRVTEDSARLSWTAPDAAFDSFFIYYIESYPAGEA |
| | IVLTVPGSERSYDLTGLKPGTEYWVGIDGVKGGRWSTPLSAIFTT |
| 42 | MLPAPKNLVVSRVTEDSARLSWTAPDAAFDSFTIEYYESFYGGEA |
| | IVLTVPGSERSYDLTGLKPGTEYYVSIYGVKGGWLSRPLSAIFTT |
| 43 | MLPAPKNLVVSRVTEDSARLSWTAPDAAFDSFYIEYYESYPGGEA |
| | IVLTVPGSERSYDLTGLKPGTEYDVYIYGVKGGYWSRPLSAIFTT |
| 44 | MLPAPKNLVVSRVTEDSARLSWTAPDAAFDSFYIEYYESLPDGEA |
| | IVLTVPGSERSYDLTGLKPGTEYAVYIYGVKGGYYSRPLSAIFTT |
| 45 | MLPAPKNLVVSRVTEDSARLSWTAPDAAFDSFAIYYLESYPEGEA |
| | IVLTVPGSERSYDLTGLKPGTEYWVGIDGVKGGTWSSPLSAIFTT |
| 46 | MLPAPKNLVVSRVTEDSARLSWTAPDAAFDSFTIEYFEFTGTGEA |
| | IVLTVPGSERSYDLTGLKPGTEYYVSIYGVKGGLLSAPLSAIFTT |
| 47 | MLPAPKNLVVSRVTEDSARLSWTAPDAAFDSFTIWYAEALGDGEA |
| | IVLTVPGSERSYDLTGLKPGTEYFVDIYGVKGGFWSLPLSAIFTT |
| 48 | MLPAPKNLVVSRVTEDSARLSWTAPDAAFDSFVIEYFEQFNLGEA |
| | IVLTVPGSERSYDLTGLKPGTEYWVGIYGVKGGWLSHPLSAIFTT |
| 49 | MLPAPKNLVVSRVTEDSARLSWTAPDAAFDSFGISYLEWWEDGEA |
| | IVLTVPGSERSYDLTGLKPGTEYWVSIAGVKGGKRSYPLSAIFTT |
| 50 | MLPAPKNLVVSRVTEDSARLSWTAPDAAFDSFVIEYREGAWYGEA |
| | IVLTVPGSERSYDLTGLKPGTEYFVDITGVKGGWWSDPLSAIFTT |
| 51 | MLPAPKNLVVSRVTEDSARLSWTAPDAAFDSFFIKYIEWWADGEA |
| | IVLTVPGSERSYDLTGLKPGTEYLVEIYGVKGGKWSWPLSAIFTT |
| 52 | MLPAPKNLVVSRVTEDSARLSWTAPDAAFDSFKISYQEWWEDGEA |
| | IVLTVPGSERSYDLTGLKPGTEYWVNISGVKGGVQSYPLSAIFTT |
| 53 | MLPAPKNLVVSRVTEDSARLSWTAPDAAFDSFFISYIEWWDLGEA |
| | IVLTVPGSERSYDLTGLKPGTEYHVEIFGVKGGTQSYPLSAIFTT |
| 54 | MLPAPKNLVVSRVTEDSARLSWTAPDAAFDSFQILYQENAFEGEA |
| | IVLTVPGSERSYDLTGLKPGTEYWVYIYGVKGGYPSVPLSAIFTT |
| 55 | MLPAPKNLVVSRVTEDSARLSWTAPDAAFDSFIIEYWEFVGYEAG |
| | IVLTVPGSERSYDLTGLKPGTEYWVAIYGVKGGDLSKPLSAIFTT |
| 56 | MLPAPKNLVVSRVTEDSARLSWTAPDAAFDSFVIEYFEALEGGEA |
| | IVLTVPGSERSYDLTGLKPGTEYFVGIYGVKGGPLSKPLSAIFTT |
| 57 | MLPAPKNLVVSRVTEDSARLSWTAPDAAFDSFSIKYLEWWQDGEA |
| | IVLTVPGSERSYDLTGLKPGTEYYVHIAGVKGGYRSYPLSAIFTT |
| 58 | MLPAPKNLVVSRVTEDSARLSWTAPDAAFDSFTIWYAEADGWGEA |
| | IVLTVPGSERSYDLTGLKPGTEYFVDIYGVKGGYLSVPLSAIFTT |
| 59 | MLPAPKNLVVSRVTEDSARLSWTAPDAAFDSFTIWYAEWEDEGEA |
| | IVLTVPGSERSYDLTGLKPGTEYRVEIYGVKGGYPSKPLSAIFTT |
| 60 | MLPAPKNLVVSRVTEDSARLSWTAPDAAFDSFTIWYAEAIGHGEA |
| | IVLTVPGSERSYDLTGLKPGTEYWVDIWGVKGGQQSKPLSAIFTT |
| 61 | MLPAPKNLVVSRVTEDSARLSWRVESRTFDSFLIQYQESEKVGEA |
| | IVLTVPGSERSYDLTGLKPGTEYTVSIYGVVWDTRDNPISNPLSA |
| | IFTT |
| 62 | MLPAPKNLVVSRVTEDSARLSWTAPDAAFDSFPILYLELNHHGEE |
| | IVLTVPGSERSYDLTGLKPGTEYWVYIFGVKGGMYSAPLSAIFTT |
| | GG |

### Example 4: Selection of Fibronectin Type III (FN3) Domains that Bind CD71

Panning and Biochemical Screening Methods for Identifying FN3 domains that bind to CD71 that do not inhibit transferrin binding to CD71. To screen for FN3 domains that specifically bind CD71 and do not inhibit transferring binding to CD71, streptavidin-coated Maxisorp plates (Nunc catalog 436110) are blocked for 1 hour in Starting Block T20 (Pierce) and then are coated with biotinylated CD71 (using same antigen as in panning) or negative controls (an unrelated Fc-fused recombinant protein and human serum albumin) for 1 hour in the presence of transferring or with FN3 protein that binds to the CD71 transferrin binding site. The concentration of transferrin is up to M. Without being bound to any particular theory, the inclusion of the transferrin or the FN3 protein that binds to the CD71 transferrin binding site pushes the selection of the FN3 domains to those that do not compete or inhibit with transferrin binding to CD71. Plates are rinsed with TBST and diluted lysate is applied to plates for 1 hour. Following additional rinses, wells are treated with HRP-conjugated anti-V5 tag antibody (Abcam, ab1325), for 1 hour and then are assayed with POD (Roche, 11582950001). The DNA from FN3 domain lysates with signals at least 10-fold ELISA signal above that of streptavidin controls are sequenced resulting in FN3 domain sequences isolated from the screening.

### Example 5: Selection of Fibronectin Type III (FN3) Domains that Bind CD71 and are not Competitive with Transferrin

To identify CD71 binding FN3 domains that were either not competitive or minimally competitive with transferrin a biased CIS-display strategy was designed. In short, using the output recovered after 5 rounds of panning on the ECD of human CD71 (Example 3). Additional rounds of off-rate selection were performed as described in Example 3 with the addition of either 1) a wash step with human holo transferrin to elute Centyrins that bound at the same site as transferrin before the final elution step or 2) elution of FN3 domain binders with monoclonal antibody OKT9. FN3 domains recovered from the transferrin wash strategy and the OKT9 elution strategy were PCR amplified and cloned into pET vector as previously described (Example 3). 228 FN3 domains that specifically bound huCD71 were confirmed by ELISA for binding to huCD71 ECD (Table 8). A subset of the unique binders was analyzed by SEC (Table 9), conjugated to MMAF and assessed for internalization via cell viability assay in SKBR-3 cells+/−holo human transferrin (Table 10). The polypeptides were found to be internalized by the receptor. The data and sequences of the hits are identified in the following tables.

TABLE 8
Summary of Screening Hits

| | | | SEQ ID |
|---|---|---|---|
| hCD71 | HSA | hCD71:HSA | NO: |
| 536550 | 28550 | 19 | 81 |
| 3525900 | 18900 | 187 | 82 |
| 1926800 | 1300 | 1482 | 83 |
| 1728500 | 3850 | 449 | 84 |
| 2912250 | 2500 | 1165 | 85 |
| 3387400 | 2250 | 1506 | 86 |
| 2833200 | 1100 | 2576 | 87 |
| 708250 | 6000 | 118 | 88 |
| 3444800 | 2000 | 1722 | 89 |
| 1093700 | 2550 | 429 | 90 |
| 1440400 | 1800 | 800 | 91 |
| 3845200 | 1300 | 2958 | 92 |
| 2162550 | 1650 | 1311 | 93 |
| 439050 | 9900 | 44 | 94 |
| 4206300 | 1900 | 2214 | 95 |
| 2714300 | 1500 | 1810 | 96 |
| 2405750 | 2200 | 1094 | 97 |
| 1159200 | 15500 | 75 | 98 |
| 853050 | 8150 | 105 | 99 |
| 2954050 | 2350 | 1257 | 100 |
| 1965650 | 9100 | 216 | 101 |
| 3476400 | 13450 | 258 | 102 |
| 4828150 | 1200 | 4023 | 103 |
| 3575700 | 1700 | 2103 | 104 |
| 1758350 | 1400 | 1256 | 105 |
| 593650 | 1200 | 495 | 106 |
| 419800 | 4050 | 104 | 107 |
| 3189250 | 1300 | 2453 | 108 |
| 4831750 | 1250 | 3865 | 109 |
| 1680700 | 18850 | 89 | 110 |
| 2399600 | 7450 | 322 | 111 |
| 3652100 | 7100 | 514 | 112 |
| 2138900 | 22550 | 95 | 113 |
| 3274950 | 4200 | 780 | 114 |
| 2917250 | 3450 | 846 | 115 |
| 536350 | 1500 | 358 | 116 |
| 1498750 | 23500 | 64 | 117 |
| 2244850 | 18850 | 119 | 118 |
| 3156200 | 1850 | 1706 | 119 |
| 3636800 | 1850 | 1966 | 120 |
| 2372350 | 28600 | 83 | 121 |
| 2305100 | 29550 | 78 | 122 |
| 707200 | 3100 | 228 | 123 |
| 605100 | 3450 | 175 | 124 |
| 2329300 | 1050 | 2218 | 125 |
| 4494550 | 1750 | 2568 | 126 |
| 3556050 | 93300 | 38 | 127 |
| 663600 | 3250 | 204 | 128 |
| 2311100 | 1500 | 1541 | 129 |
| 1446000 | 6050 | 239 | 130 |
| 2183100 | 2400 | 910 | 131 |
| 2747200 | 2350 | 1169 | 132 |
| 4277350 | 5000 | 855 | 133 |
| 3080150 | 1950 | 1580 | 134 |
| 2056200 | 1500 | 1371 | 135 |
| 4026050 | 5700 | 706 | 136 |
| 484350 | 2050 | 236 | 137 |
| 4178200 | 3500 | 1194 | 138 |
| 3034550 | 2100 | 1445 | 139 |
| 4175400 | 1600 | 2610 | 140 |
| 2041000 | 3150 | 648 | 141 |
| 3413400 | 3400 | 1004 | 142 |
| 3940800 | 9250 | 426 | 143 |
| 2711900 | 2750 | 986 | 144 |
| 1615500 | 48450 | 33 | 145 |
| 3074850 | 3800 | 809 | 146 |
| 2363750 | 71250 | 33 | 147 |
| 3976550 | 2000 | 1988 | 148 |
| 2768350 | 3350 | 826 | 149 |
| 2617600 | 3500 | 748 | 150 |
| 1770200 | 54950 | 32 | 151 |
| 2831150 | 5300 | 534 | 152 |
| 956700 | 13100 | 73 | 153 |
| 2779300 | 4150 | 670 | 154 |
| 1837850 | 25950 | 71 | 155 |
| 1028500 | 5000 | 206 | 156 |
| 2657950 | 2450 | 1085 | 157 |
| 2055750 | 1350 | 1523 | 158 |
| 2581000 | 1950 | 1324 | 159 |
| 2759200 | 3300 | 836 | 160 |
| 1214400 | 5050 | 240 | 161 |
| 3876250 | 1850 | 2095 | 162 |
| 3047800 | 4400 | 693 | 163 |
| 2605000 | 1100 | 2368 | 164 |
| 2642300 | 65200 | 41 | 165 |
| 2421600 | 5050 | 480 | 166 |
| 2618650 | 3600 | 727 | 167 |
| 2896650 | 1950 | 1485 | 168 |
| 2853900 | 2700 | 1057 | 169 |
| 981650 | 16800 | 58 | 170 |
| 3720500 | 1900 | 1958 | 171 |
| 4309800 | 3700 | 1165 | 172 |
| 979050 | 20850 | 47 | 173 |
| 2422100 | 2200 | 1101 | 174 |
| 3550650 | 1600 | 2219 | 175 |
| 1336350 | 10350 | 129 | 176 |
| 2608650 | 2450 | 1065 | 177 |
| 1447950 | 2250 | 644 | 178 |
| 2684550 | 74700 | 36 | 179 |
| 1678750 | 33500 | 50 | 180 |
| 2945100 | 6000 | 491 | 181 |
| 3116750 | 2950 | 1057 | 182 |
| 1724000 | 10700 | 161 | 183 |
| 470400 | 2000 | 235 | 184 |
| 1809500 | 9950 | 182 | 185 |
| 2024550 | 1850 | 1094 | 186 |
| 3061100 | 7250 | 422 | 187 |
| 2669350 | 3350 | 797 | 188 |
| 1962850 | 67500 | 29 | 189 |
| 3214200 | 4900 | 656 | 190 |
| 1465100 | 33950 | 43 | 191 |
| 2666650 | 4100 | 650 | 192 |
| 3872950 | 2500 | 1549 | 193 |
| 562350 | 17800 | 32 | 194 |
| 2532200 | 90200 | 28 | 195 |
| 1719750 | 34550 | 50 | 196 |
| 4566550 | 18900 | 242 | 197 |
| 3441600 | 3050 | 1128 | 198 |
| 1461350 | 14450 | 101 | 199 |
| 3626550 | 2100 | 1727 | 200 |
| 1197600 | 11350 | 106 | 201 |
| 4503050 | 2800 | 1608 | 202 |
| 3382850 | 3300 | 1025 | 203 |
| 2766650 | 24550 | 113 | 204 |
| 434050 | 3350 | 130 | 205 |
| 833350 | 2650 | 314 | 206 |
| 1596550 | 25600 | 62 | 207 |
| 2289200 | 46700 | 49 | 208 |
| 790150 | 13750 | 57 | 209 |
| 1156900 | 2250 | 514 | 210 |
| 1001850 | 3000 | 334 | 211 |
| 2490750 | 2250 | 1107 | 212 |
| 2105500 | 9800 | 215 | 213 |
| 2143100 | 2200 | 974 | 214 |
| 2125250 | 1750 | 1214 | 215 |
| 2192150 | 21800 | 101 | 216 |
| 3902700 | 11750 | 332 | 217 |
| 2388200 | 33800 | 71 | 218 |
| 3307550 | 2600 | 1272 | 219 |
| 4247800 | 9400 | 452 | 220 |
| 1959700 | 3650 | 537 | 221 |
| 1741200 | 3950 | 441 | 222 |
| 1666800 | 51950 | 32 | 223 |
| 2017650 | 16500 | 122 | 224 |
| 2962400 | 14100 | 210 | 225 |
| 4332150 | 2850 | 1520 | 226 |
| 3853700 | 2300 | 1676 | 227 |
| 2542750 | 2400 | 1059 | 228 |
| 570000 | 3700 | 154 | 229 |
| 1998400 | 1900 | 1052 | 230 |
| 2268400 | 26500 | 86 | 231 |
| 1699150 | 2700 | 629 | 232 |
| 3412150 | 2600 | 1312 | 233 |
| 680200 | 7200 | 94 | 234 |
| 3923600 | 1350 | 2906 | 235 |
| 3444750 | 1500 | 2297 | 236 |
| 4148900 | 1850 | 2243 | 237 |
| 2883800 | 4250 | 679 | 238 |
| 418900 | 5050 | 83 | 239 |
| 3033700 | 2050 | 1480 | 240 |
| 2696100 | 2200 | 1226 | 241 |
| 871750 | 4900 | 178 | 242 |
| 2402150 | 10150 | 237 | 243 |
| 545300 | 1650 | 330 | 244 |
| 2617750 | 2900 | 903 | 245 |
| 1573350 | 1400 | 1124 | 246 |
| 916150 | 53050 | 17 | 247 |
| 831650 | 15100 | 55 | 248 |
| 1047250 | 7100 | 148 | 249 |
| 1094500 | 18750 | 58 | 250 |
| 2738000 | 9650 | 284 | 251 |
| 2979550 | 2500 | 1192 | 252 |
| 2801100 | 2450 | 1143 | 253 |
| 3243550 | 90000 | 36 | 254 |
| 1835800 | 4550 | 403 | 255 |
| 1978900 | 2200 | 900 | 256 |
| 2374200 | 3950 | 601 | 257 |
| 1041700 | 10600 | 98 | 258 |
| 2443600 | 2100 | 1164 | 259 |
| 1301700 | 14450 | 90 | 260 |
| 4233400 | 5550 | 763 | 261 |
| 4380350 | 2350 | 1864 | 262 |
| 1878900 | 20400 | 92 | 263 |
| 2977200 | 2550 | 1168 | 264 |
| 3606650 | 3950 | 913 | 265 |
| 894150 | 2650 | 337 | 266 |
| 1969550 | 11900 | 166 | 267 |
| 1597000 | 2550 | 626 | 268 |
| 690150 | 4150 | 166 | 269 |
| 1809350 | 2400 | 754 | 270 |
| 2114700 | 3050 | 693 | 271 |
| 1784450 | 8950 | 199 | 272 |
| 4651050 | 9150 | 508 | 273 |
| 522300 | 11900 | 44 | 274 |
| 2245050 | 3800 | 591 | 275 |
| 720100 | 12350 | 58 | 276 |
| 3110300 | 5200 | 598 | 277 |
| 3689600 | 6500 | 568 | 278 |
| 4089350 | 3150 | 1298 | 279 |
| 445950 | 21550 | 21 | 280 |
| 1073150 | 22400 | 48 | 281 |
| 3851150 | 18650 | 206 | 282 |
| 2952800 | 6250 | 472 | 283 |
| 2901100 | 5250 | 553 | 284 |
| 2435900 | 2200 | 1107 | 285 |
| 1270750 | 10750 | 118 | 286 |
| 3882900 | 5500 | 706 | 287 |
| 658700 | 40800 | 16 | 288 |
| 2268450 | 2150 | 1055 | 289 |
| 2810350 | 11850 | 237 | 290 |
| 3829050 | 2150 | 1781 | 291 |
| 2620700 | 8850 | 296 | 292 |
| 3588450 | 6900 | 520 | 293 |
| 1436450 | 8250 | 174 | 294 |
| 3384850 | 3800 | 891 | 295 |
| 2701450 | 3200 | 844 | 296 |
| 2594250 | 52550 | 49 | 297 |
| 2514000 | 34050 | 74 | 298 |
| 4270100 | 2200 | 1941 | 299 |
| 2311150 | 4050 | 571 | 300 |
| 659800 | 12050 | 55 | 301 |
| 2672850 | 21200 | 126 | 302 |
| 3513150 | 2650 | 1326 | 303 |
| 3343900 | 2700 | 1238 | 304 |
| 1207900 | 25000 | 48 | 305 |
| 4068850 | 2250 | 1808 | 306 |
| 2185950 | 4350 | 503 | 307 |
| 608900 | 12300 | 50 | 308 |
| 3142450 | 2850 | 1103 | 309 |

TABLE 9
Summary of Size Exclusion Chromatography Analysis

| SEQ ID | RT | Height |
|---|---|---|
| NO: | (min) | (mAU) |
| 81 | 5.117 | 14621 |
| 82 | 5.11 | 24062 |
| 83 | 5.114 | 91333 |
| 84 | 5.032 | 65838 |
| 85 | 5.075 | 78484 |
| 86 | 5.149 | 210493 |
| 87 | 5.1 | 77812 |
| 88 | 5.14 | 194249 |
| 89 | 5.006 | 61555 |
| 90 | 5.071 | 177756 |
| 91 | 5.092 | 127220 |
| 92 | 5.217 | 179747 |
| 93 | 5.043 | 35064 |
| 94 | 6.706 | 2222 |
| 95 | 5.112 | 75615 |
| 96 | 5.066 | 71880 |
| 97 | 5.144 | 101200 |
| 98 | 4.561 | 29769 |
| 99 | 3.764 | 3242 |
| 100 | 5.158 | 163566 |
| 101 | 5.049 | 70310 |
| 102 | 5.06 | 48409 |
| 103 | 5.047 | 85919 |
| 104 | 5.04 | 67751 |
| 105 | 5.076 | 79635 |
| 106 | 5.092 | 100250 |
| 107 | 3.755 | 3878 |
| 108 | 5.131 | 109212 |
| 109 | 5.048 | 72864 |
| 110 | 5.037 | 25838 |
| 111 | 5.046 | 82613 |
| 112 | 5.037 | 69662 |
| 113 | 5.06 | 1660 |
| 114 | 5.058 | 93289 |
| 115 | 5.008 | 59386 |
| 116 | 6.701 | 78 |
| 117 | 5.001 | 16853 |
| 119 | 5.026 | 49470 |
| 120 | 5.247 | 131571 |
| 121 | 4.494 | 4134 |
| 122 | 4.576 | 20348 |
| 123 | 4.572 | 16021 |
| 124 | 5.018 | 69849 |
| 125 | 5.007 | 69810 |
| 126 | 5.075 | 64475 |
| 127 | 5.07 | 12214 |
| 128 | 5.107 | 58225 |
| 129 | 5.005 | 122592 |
| 130 | 5.051 | 116931 |
| 131 | 5.073 | 95190 |
| 132 | 5.038 | 106856 |
| 133 | 5.082 | 20172 |
| 134 | 5.118 | 97944 |
| 135 | 5.032 | 97600 |
| 136 | 5.157 | 66595 |
| 137 | 5.032 | 156482 |
| 138 | 5.181 | 124800 |
| 139 | 4.978 | 96486 |
| 140 | 5.024 | 78145 |
| 141 | 5.095 | 115919 |
| 142 | 5.067 | 52467 |
| 143 | 5.042 | 50518 |
| 144 | 5.062 | 82962 |
| 145 | 4.542 | 18503 |
| 146 | 5.031 | 88958 |
| 147 | 4.509 | 8929 |
| 148 | 5.098 | 91401 |
| 149 | 5.055 | 79364 |
| 150 | 4.976 | 57089 |
| 151 | 4.469 | 10958 |
| 152 | 5.017 | 67201 |
| 153 | 5.108 | 89015 |
| 154 | 5.083 | 73990 |
| 155 | 4.57 | 3820 |
| 156 | 5.053 | 125648 |
| 157 | 5.131 | 96835 |
| 158 | 4.964 | 86205 |
| 159 | 4.994 | 66919 |
| 160 | 5.11 | 94133 |
| 161 | 5.018 | 103592 |
| 162 | 5.157 | 96072 |
| 163 | 5.049 | 121129 |
| 164 | 5.115 | 79403 |
| 165 | 4.547 | 6562 |
| 166 | 5.023 | 125865 |
| 167 | 4.975 | 63859 |
| 168 | 5.043 | 86853 |
| 169 | 5.017 | 95640 |
| 170 | 5.04 | 54100 |
| 171 | 5.18 | 180492 |
| 172 | 5.229 | 70453 |
| 173 | 3.662 | 17075 |
| 174 | 4.999 | 268853 |
| 175 | 5.044 | 272743 |
| 178 | 5.063 | 40232 |
| 179 | 5.11 | 233798 |
| 180 | 5.028 | 268714 |
| 181 | 5.049 | 175217 |
| 182 | 5.024 | 347191 |
| 183 | 5.161 | 269305 |
| 184 | 4.967 | 236502 |
| 185 | 5.018 | 190752 |
| 186 | 5.081 | 342318 |
| 187 | 5.038 | 127542 |
| 188 | 5.043 | 140513 |
| 189 | 5.058 | 218023 |
| 190 | 4.535 | 55627 |
| 191 | 5.026 | 199881 |
| 192 | 4.708 | 31553 |
| 193 | 5.086 | 1933389 |
| 194 | 5.046 | 253626 |
| 195 | 4.969 | 143010 |
| 196 | 4.996 | 80332 |
| 197 | 5.009 | 141197 |
| 198 | 5.1 | 139202 |
| 199 | 5.126 | 123977 |
| 200 | 5.449 | 1886 |
| 201 | 5.047 | 226703 |
| 203 | 4.955 | 172346 |
| 204 | 4.987 | 159535 |
| 205 | 5.09 | 237874 |
| 206 | 5.01 | 182142 |
| 207 | 5.144 | 190642 |
| 208 | 5.034 | 190328 |
| 209 | 5.104 | 221965 |
| 210 | 5053 | 5060 |
| 211 | 5.009 | 287859 |
| 212 | 4.969 | 187947 |
| 213 | 5.026 | 219651 |
| 214 | 4.999 | 181968 |
| 215 | 5.034 | 111935 |
| 216 | 5.158 | 401933 |
| 217 | 5.197 | 275205 |
| 218 | 4.447 | 74121 |
| 219 | 4.97 | 215336 |
| 220 | 5.051 | 260942 |
| 221 | 4.957 | 123233 |
| 222 | 5.03 | 1674429 |
| 223 | 5.012 | 145280 |
| 224 | 5.534 | 2310 |
| 225 | 5.017 | 54242 |
| 226 | 5.001 | 142955 |
| 227 | 5.024 | 212808 |
| 228 | 5.039 | 1149, 33 |
| 229 | 5.064 | 177947 |
| 230 | 4.983 | 202000 |
| 231 | 5.013 | 182975 |
| 232 | 5.121 | 223657 |
| 233 | 5.092 | 172952 |
| 234 | 3.951 | 84866 |
| 235 | 5.058 | 142138 |
| 236 | 5.063 | 367688 |
| 237 | 5.004 | 165516 |
| 238 | 5.069 | 218298 |
| 239 | 5.086 | 361567 |
| 240 | 5.127 | 252675 |
| 241 | 5.071 | 233781 |
| 242 | 5.008 | 268637 |
| 243 | 5.092 | 168008 |
| 244 | 5.119 | 79488 |
| 245 | 5.06 | 215547 |
| 246 | 5.008 | 53653 |
| 247 | 5.075 | 250310 |
| 248 | 5.094 | 194793 |
| 249 | 3.616 | 37488 |
| 250 | 5.036 | 301239 |
| 251 | 5.101 | 297658 |
| 252 | 4.965 | 53405 |
| 253 | 4.65 | 4466 |
| 254 | 3.66 | 16463 |
| 255 | 5.032 | 253885 |
| 256 | 4.976 | 244457 |
| 257 | 5.072 | 289009 |
| 258 | 5.106 | 273939 |
| 259 | 5.041 | 166066 |
| 260 | 5.004 | 160654 |
| 261 | 4.972 | 164451 |
| 262 | 5.148 | 513577 |
| 263 | 5.089 | 208950 |
| 264 | 5.099 | 206909 |
| 265 | 5.051 | 68567 |
| 266 | 4.996 | 72025 |
| 267 | 5.085 | 106826 |
| 268 | 4.865 | 7221 |
| 269 | 5.138 | 63713 |
| 270 | 5.186 | 149808 |
| 271 | 5.019 | 85191 |
| 272 | 5.277 | 118699 |
| 273 | 5.069 | 104693 |
| 274 | 5.022 | 17776 |
| 275 | 5.055 | 138448 |
| 276 | 4.95 | 16306 |
| 277 | 5.079 | 139094 |
| 278 | 5 | 82052 |
| 279 | 5.088 | 3310 |
| 280 | 5.22 | 127670 |
| 281 | 5.039 | 157800 |
| 282 | 5.003 | 109468 |
| 283 | 5.074 | 123519 |
| 284 | 5.039 | 12331 |
| 285 | 5.223 | 148145 |
| 286 | 5.136 | 148676 |
| 287 | 3.665 | 7404 |
| 288 | 5.575 | 1112 |
| 289 | 3.696 | 9460 |
| 290 | 5.029 | 93755 |
| 291 | 5.095 | 169623 |
| 292 | 3.689 | 14445 |
| 293 | 4.634 | 36542 |
| 294 | 5.004 | 77308 |
| 295 | 4.998 | 17822 |
| 296 | 5.003 | 74551 |
| 297 | 5.085 | 68904 |
| 298 | 5.192 | 129131 |
| 299 | 4.54 | 30337 |
| 300 | 5.025 | 142111 |
| 301 | 5.028 | 84156 |
| 302 | 4.992 | 78611 |
| 303 | 4.527 | 25755 |
| 304 | 5.065 | 122824 |
| 305 | 3.668 | 7392 |
| 306 | 5.065 | 145979 |
| 307 | 5.097 | 135403 |
| 308 | 5.059 | 18037 |
| 309 | 5.198 | 111922 |

TABLE 10
IC50 of CD71 FN3 domain - MMAF conjugate molecules 5 on
SKBR-3 cells +/− human transferrin

| SEQ ID NO: | IC50 (nM) | |
|---|---|---|
| 146 | 7.131 | 3.457 |
| 214 | 28.15 | 29.23 |
| 104 | 301.3 | 5.9 |
| 259 | 27.46 | N.D. |
| 134 | 164.9 | 8.543 |
| 92 | 5.489 | 1.061 |
| 302 | 164.3 | 27.81 |
| 235 | 1.755 | 10.58 |
| 237 | 28.12 | 3.762 |
| 152 | 19.56 | 5.239 |
| 238 | N.D. | 7.232 |
| 136 | 2.32 | 0.5026 |
| 197 | N.D. | 0.4675 |
| 212 | N.D. | 6.691 |
| 296 | 29.31 | 18.61 |
| 226 | N.D. | 8.32 |
| 261 | 1.235 | 31.2 |
| 307 | 47.89 | 30.75 |
| 115 | 24.22 | 10.43 |
| 112 | 27.33 | 4.549 |
| 278 | 13.24 | 3.702 |
| 297 | N.D. | N.D. |
| 96 | 79.5 | 27 |
| 222 | N.D. | 28.23 |
| 95 | 28.27 | 12.68 |
| 233 | 54.61 | 17.7 |
| 217 | 15.78 | 2.458 |
| 252 | 24.55 | 7.736 |
| 194 | N.D. | 5.091 |
| 164 | 18.7 | 55.8 |
| 168 | 32 | 7.2 |
| 174 | ND | 158 |
| 190 | 36 | 12 |
| 257 | 22 | 6.5 |
| 303 | 33 | 39 |
| 284 | 98 | 32 |
| 85 | ND | 89 |
| 149 | 9.7 | 5.5 |

| SEQ | |
|---|---|
| ID | Amino Acid sequence of FN3 domains |
| NO: | that bind to CD71 |
| 81 | MLPAPKNLVVSRVTEDSARLSWTAPDAAFDSFVIEYREGAWYGEA |
| | IVLTVPGSERSYDLTGLKPGTEYAVYIPGVKGGPRSFPLSAIFTT |
| 82 | MLPAPKNLVVSRVTEDSARLSWTAPDAAFDSFAIAYVEWWKLGEA |
| | IVLTVPGSERSYDLTGLKPGTEYVVPIPGVKGGGHSSPLSAIFTT |
| 83 | MLPAPKNLVVSRVTEDSARLSWTAPDAAFDSFYIYYYESSGTGEA |
| | IVLTVPGSERSYDLTGLKPGTEYFVDIGGVKGGSYSLPLSAIFTT |
| 84 | MLPAPKNLVVSRVTEDSARLSWTAPDAAFDSFPIYYWEVFPAGEA |
| | IELDVPGSERSYDLTGLKPGTEYFVRIEGVKGGASSYPLRAEFTT |
| 85 | MLPAPKNLVVSRVTEDSARLSWTAPDAAFDSFFIWYWEKSVDGEA |
| | IVLTVPGSERSYDLTGLKPGTEYNVGIQGVKGGTPSDPLSAIFTT |
| 86 | MLPAPKNLVVSRVTEDSARLSWTAPDAAFDSFIIWYAEWVNDGEA |
| | IVLTVPGSERSYDLTGLKPGTEYRVEITGVKGGTWSRPLSAIFTT |
| 87 | MLPAPKNLVVSRVTEDSARLSWTAPDAAFDSFAIEYYEPVPAGEA |
| | IYLDVPGSERSYDLTGLKPGTEYDVTIYGVKGGYYSHPLFASFTT |
| 88 | MLPAPKNLVVSRVTEDSARLSWTAPDAAFDSFWIEYFEWTVGGEA |
| | IVLTVPGSERSYDLTGLKPGTEYYVSIYGVKGGWLSPPLSAIFTT |
| 89 | MLPAPKNLVVSRVTEDSARLSWTAPDAAFDSFHISYEETPVVGEA |
| | IYLRVPGSERSYDLTGLKPGTEYTVAIHGVKGGRESTPLIAPFTT |
| 90 | MLPAPKNLVVSRVTEDSARLSWTAPDAAFDSFTIHYWEFDPPGEA |
| | IVLTVPGSERSYDLTGLKPGTEYTVYIEGVKGGWWSKPLSAIFTT |
| 91 | MLPAPKNLVVSRVTEDSARLSWTAPDAAFDSFVIEYWERTQPGEA |
| | IVLTVPGSERSYDLTGLKPGTEYDVWISGVKGGKWSEPLSAIFTT |
| 92 | MLPAPKNLVVSRVTEDSARLSWTAPDAAFDSFLIRYWEWYVLGEA |
| | IVLTVPGSERSYDLTGLKPGTEYYVEISGVKGGWQSWPLSAIFTT |
| 93 | MLPAPKNLVVSRVTEDSARLSWTAPDAAFDSFYIGYLEPGDNGEA |
| | IVLTVPGSERSYDLTGLKPGTEYNVSIGGVKGGLGSYPLSAIFTT |
| 94 | MLPAPKNLVVSRITEDSARLSWTAPDAAFDSFGIYYYEWWSTGEA |
| | IVLTVPGSERSYDLTGPKPGTEYYVKISGVKGGYRSYPLSAIFTT |
| 95 | MLPAPKNLVVSRVTEDSARLSWTAPDAAFDSFRISYYEWYDLGEA |
| | IVLTVPGSERSYDLTGLKPGTEYWVDIAGVKGGYYSYPLSAIFIT |
| 96 | MLPAPKNLVVSRVTEDSARLSWTAPDAAFDSFFIGYLEPQPPGEA |
| | IVLTVPGSERSYDLTGLKPGTEYNVTIQGVKGGFPSMPLSAIFTT |
| 97 | MLPAPKNLVVSRVTEDSARLSWTAPDAAFDSFFISYFEGWASGEA |
| | IHLYVPGSERSYDLTGLKPGTEYSVHIQGVKGGQPSTPLSAIFTT |
| 98 | MLPAPKNLVVSRITEDSARLSWTAPDAAFDSFDIPYGEFDTIGEA |
| | IVLTVPGSERSYDLTGLKPGTEYDVYIEGVKGGHLSWPLSAIFTT |
| 99 | MLPAPKNLVVSRVTEDSARLSWTAPDAAFDSFGIQYNEFVFRGEA |
| | IVLTVPGSERSYDLTGLKPGTEYFVPISGVKGGDDSRPLSAIFTT |
| 100 | MLPAPKNLVVSRVTEDSARLSWTAPDAAFDSFWIEYWEVVGFGEA |
| | IVLTVPGSERSYDLTGLKPGTEYWVGIYGVKGGNPSVPLSAIFTT |
| 101 | MLPAPKNLVVSRVTEDSARLSWTAPDAAFDSFAIDYDEPINSGEA |
| | IVLTVPGSERSYDLTGPKPGTEYEVEIYGVKGGYLSRPLSAIFTT |
| 102 | MLPAPKNLVVSRVTEDSARLSWTAPDAAFDSFSIDYDEPQPVGEA |
| | IVLTVPGSERSYDLTGLKPGTEYRVDIWGVKGGPTSGPLRATFTT |
| 103 | MLLAPKNLVVSRVTEDSARLSWTAPDAAFDSFAIEYFEYTGEGEA |
| | IVLTVPGSERSYDLTGLKPGTEYYVGIYGVKGGYLSRPLSAIFTT |
| 104 | MLPAPKNLVVSHVTEDSARLSWTAPDAAFDSFDIEYYELVGSGEA |
| | IVLTVPGSERSYDLTGLKPGTEYYVAIYGVKGGYLSRPLSAIFTT |
| 105 | MLPAPKNLVVSRVTEDSARLSWTAPDAAFDSFGIAYYERSGAGEA |
| | IVLTVPGSERSYDLTGLKPGTEYMVYINGVKGGFVSSPLSAIFTT |
| 106 | MLPAPKNLVVSRVTEDSARLSWTAPDAAFDSFTIAYEEHGLVGEA |
| | IYLRVPGSERSYDLTGLKPGTEYHVGIMGVKGGVFSSPLSAIFTT |
| 107 | MLPAPKNLVVSRVTEDSARLSWTAPDAAFDSFFIQYTESHWVGEA |
| | IVLTVPGSERSYDLTGLKPGTEYAVPIEGVKGGDSSTPLSAIFTT |
| 108 | MLPAPKNLVVSRVTEDSARLSWTAPDAAFDSFTIIYGEVNPYGEA |
| | IVLTVPGSERSYDLTGLKPGTEYDVFIEGVKGGHLSWPLSAIFTT |
| 109 | MLPAPKNLVVSRVTEDSARLSWTAPDAAFDSFHIAYEELVTEGEA |
| | IYLRVPGSERSYDLTGLKPGTEYLVDIEGVKGGHLSSPLSAIFTT |
| 110 | MLPAPKNLVVSRVTEDSARLSWTAPDAAFDSFFIHYHEWWEAGEA |
| | IVLTVPGSERSYDLTGLKPGTEYLVDIPGVKGGDLSVPLSAIFTT |
| 111 | MLPAPKNLVVSRVTEDSARLSWTAPDAAFDSFWIYYYESVGTGEA |
| | IVLTVPGSERSYDLTGLKPGTEYFVDISGVKVGTYSLPLSAIFTT |
| 112 | MLPAPKNLVVSRVTEDSARLSWTAPDAAFDSFFIAYFEFANPGEA |
| | IVLTVPGSERSYDLTGLKPGTEYKVVIQGVKGGTPSEPLSAISTT |
| 113 | MLPAPKNLVVSRVTEDSARLSWTAPDAAFDSFDIHYKEHSWWGEA |
| | IVLTVPGSERSYDLTGLKPGTEYIVPIPGVKGGGISRPLSAIFTT |
| 114 | MLPAPKNLVVSRVTEDSARLSWTAPDAAFDSFAIEYWEAVGSGEA |
| | IVLTVPGSERSYDLTGLKPGTEYHVYIYGVKGGYLSLPLSAIFTT |
| 115 | MLPAPKNLVVSRVTEDSARLSWTAPDAAFDSFFIGYLEPQPPGEA |
| | IVLTVPGSERSYDLTGLKPGTEYNVTIQGVKGGFPSMPLSAIFTT |
| | T |
| 116 | MLPAPKNLVVSRVTEDSARLSWTAPDAAFDSFSIAYSEVRYDGEA |
| | IVLTVPGSERSYDLTGLKPGTEYVVPIGGVKGGGSSSPLSAIFTT |
| 117 | MLPAPKNLVVSRVTEDSARLSWTAPDAAFDSFTIPYGEAFNPGEA |
| | IVLTVPGSERSYDLTGLKPGTEYDVFIEGVKGGTLSWPLSAIFTT |
| 118 | MLPAPKNLVVSRVTEDSARLSWTAPDAAFDSFRILYGEVDPWGEA |
| | IVLTVPGSERSYDLTGLKPGTEYDVWIEGVKGGKLSWPLSAIFTT |
| 119 | MLPAPKNLVVSRVTEDSARLSWTAPDAAFDSFHIEYEETPQKGEA |
| | IFLRVPGSERSYDLTGLKPGTEYVVNIRGVKGGDLSSPLGALFTT |
| 120 | MLPAPKNLVVSRVTEDSARLSWTAPDAAFDSFRIEYIEWWVGGEA |
| | IVLTVPGSERSYDLTGLKPGTEYWVDIKGVKGGKRSYPLSAIFTT |
| 121 | MLPAPKNLVVSRVTEDSARLSWTAPDAAFDSFSIDYPEFPVRGEA |
| | IVLTVPGSERSYDLTGPKPGTEYNVTIQGVKGGFPSMPLSAIFTT |
| 122 | MLPAPKNLVVSRVTEDSARLSWTAPDAAFDSFQIPYWEQSLGGEA |
| | IVLTVPGSERSYDLTGLKPGTEYEVWIEGVKGGDLSFPLSAISTT |
| 123 | MLPAPKNLVVSRVTEDSARLSWTAPDAAFDSFVIPYEEYLYTGEA |
| | IVLTVPGSERSYDLTGLKPGTEYDVWIEGVKGGLTSWPLSAIFTT |
| 124 | MLPAPKNLVVSRVTEDSARLSWTAPDAAFDSFSIDYPEFPVRGEA |
| | IVLTVPGSERSYDLTGLKPGTEYAVTIWGVKGGFTSQPLSAIFTT |
| 125 | MLPAPKNLVVSRVTEDSARLSWTAPDAAFDSFVIEYFEFVGEGEA |
| | IVLTVPGSERSYDLTGLKPGTEYDVGIYGVKGGSLSSPLSAIFTT |
| 126 | MLPAPKNLVVSRVTEDSARLSWTAPDAAFDSFSIDYLELGESGEA |
| | IVLTVPGSERSYDLTGLKPGTEYWVYIFGVKGGYPSAPLSAIFTT |
| 127 | MLPAPKNLVVSRVTEDSARLSWTAPDAAFDSFPIPYGESPPSGEA |
| | IVLTVPGSERSYDLTGLKPGTEYVVIIRGVKGGGRSGPLSAISTT |
| 128 | MLPAPKNLVVSRVTEDSARLSWTAPDAAFDSFIINYIEIVQYGEA |
| | IVLTVPGSERSYDLTGLKPGTEYPESIWGVKGGGASSPLSAIFTT |
| 129 | MLPAPKNLVVSRVTEDSARLSWTAPDAAFDSFDIEYYEAVGAGEA |
| | IVLTVPGSERSYDLTGLKPGTEYTVGIYGVKGGWLSKPLSVIFTT |
| 130 | MLPAPKNLVVSRVTEDSARLSWTAPDAAFDSFHIPYVEAEVPGEA |
| | IQLHVPGSERSYDLTGLKPGTEYYVEIWGVKGGFYSPPLIAEFTT |
| 131 | MLPAPKNLVVSRVTEDSARLSWTAPDAAFDSFSIDYYEGKGYGEA |
| | IVLTVPGSERSYDLTGLKPGTEYQVLISGVKGGKYSLPLSAIFTT |
| 132 | MLPAPKNLVVSRVTEDSARLSWTAPDAAFDSFTIVYAEVTYDGEA |
| | IVLTVPGSERSYDLTGLKPGTEYDVFIEGVKGGELSWPLSAIFTT |
| 133 | MLPAPKNLVVSRVTEDSARLSWTAPDAAFDSFPIVYGEAWVTGEA |
| | IVLTVPGSERSYDLTGLKPGTEYDVWIEGVKGGELSWPLSAIFTT |
| 134 | MLPAPKNLVVSRVTEDSARLSWTAPDAAFDSFWIDYYERKYVGEA |
| | IVLTVPGSERSYDLTGLKPGTEYEVTIYGVKGGWYSDPLSAIFTT |
| 135 | MLPAPKNLVVSRVTEDSARLSWTAPDAAFDSFPISYYEMSGLGEA |
| | IVLTVPGSERSYDLTGLKPGTEYMVYIFGVKGGLNSLPLSAIFTT |
| 136 | MLPAPKNLVVSRVTEDSARLSWTAPDAAFDSFFIYYIESYPAGEA |
| | IVLTVPGSERSYDLTGLKPGTEYWMGIDGVKGGRWSTPLSAIFTT |
| 137 | MLPAPKNLVVSRVTEDSARLSWTAPDAAFDSFEIEYDEPSVAGEA |
| | IVLTVPGSERSYDLTGLKPGTEYRVFIWGVKGGNQSWPLSAIFTT |
| 138 | MLPAPKNLVVSRVTEDSARLSWTAPDAAFDSFFIKYIEWWADGEA |
| | IVLTVPGSERSYDLTGLKPGTEYLVEIYGVKGGRQSYPLSAIFTT |
| 139 | MLPAPKNLVVSRVTEDSARLSWTAPDAAFDSFDISYWESGKYGEA |
| | IVLTVPGSESSYDLTGLKPGTEYLVDIFGVKGGYPSEPLSAIFTT |
| 140 | MLPAPKNLVVSRVTEDSARLSWTAPDAAFDSFWISYEESDTEGEA |
| | IYLRVPGSERSYDLTGLKPGTEYNVTIQGVKGGFPSMPLSAIFTT |
| 141 | MLPAPKNLVVSRVTEDSARLSWTAPDAAFDSFVIEYFEQFNLGEA |
| | IVLTVPGSERSYDLTGLKPGTEYLVGIYGVKGGWLSHPLSAIFTT |
| 142 | MLPAPKNLVVSRVTKDSARLSWTAPDAAFDSFHIAYEEATTYGEA |
| | IFLRVPGSERSYDLTGLKPGTEYEVKIHGVKGGADSKPLVAPFTT |
| 143 | MLPAPKNLVVSRVTEDSARLSWTAPDAAFDSFHIAYEEADSEGEA |
| | IYLRVPGSERSYDLTGLKPGTEYSVNIQGVKGGIVSFPLHAEFTT |
| 144 | MLPAPKNLVVSRVTEDSARLSWTAPDAAFDSFIIPYAEVRPDGEA |
| | IVLTVPGSERSYDLTGLKPGTEYSVLIHGVKGGKLSLPLSAIFTT |
| 145 | MLPAPKNLVVSRVTEDSARLSWTAPDAAFDSFFIPYAEPSPTGEA |
| | IVLTVPGSERSYDLTGLKPGTEYDVWIEGVKGGTLSWPLSAIFTT |
| 146 | MLPAPKNLVVSRVTEDSARLSWTAPDAAFDSFYIAYAEPRPDGEA |
| | IVLTVPGSERSYDLTGLKPGTEYSVLIHGVKGGLLSSPLSAIFTT |
| 147 | MLPAPKNLVVSRVTEDSARLSWTAPDAAFDSFFIGYLEPQPPGEA |
| | IVLTVPGSERSYDLTGLKPGTEYSVLIHGVKGGRNSDPLSAISTT |
| 148 | MLPAPKNLVVSRVTEDSARLSWTAPDAAFDSFHIEYEEQYSTGEA |
| | IYLRVPGSERSYDLTGLKPGTEYHVDIEGVKGGRRSFPLNAFFTT |
| 149 | MLPAPKNLVVSRVTEDSARLSWTAPDAAFDSFIIPYAEVRPDGEA |
| | IVLTVPGSERSYDLTGLKPGTEYSVLIHGVKGGKLSEPLSAIFTT |
| 150 | MLPAPKNLVVSRVTEDSARLSWTAPDAAFDSFPIAYAEPSPTGEA |
| | IVLTVPGSERSYDLTGLKPGTEYSVLIHGVKGGHLSDPLSAIFTT |
| 151 | MLPAPKNLVVSRVTEDSARLSWTAPDAAFDSFFIPYAEPSPTGEA |
| | IVLTVPGSERSYDLTGLKPGTEYGVVILGVKGGYGSDPLSAIFTT |
| 152 | MLPAPKNLVVSRVTEDSARLSWTAPDAAFDSFFIGYLEPQPPGEA |
| | IVLTVPGSERSYDLTGLKPGTEYNVTIQGVKGGFPSSPLSAIFTT |
| 153 | MLPAPKNLVVSRVTEDSARLSWTAPDAALDSFRIAYTEYFVGGEA |
| | IVLTVPGSERSYDLTGLKPGTEYGVGIYGVKGGAGSSPLSAIFTT |
| 154 | MLPAPKNLVVSRVTEDSARLSWTAPDAAFDSFFIPYAEPRPDGEA |
| | IVLTVPGSERSYDLTGLKPGTEYSVLIHGVKGGLLSSPLSAIFTT |
| 155 | MLPAPKNLVVSRVTEDSARLSWTAPDAAFDSFPITYRERSQYGEA |
| | IVLTVPGSERSYDLTGLKPGTEYVVPIEGVKGGRGSKPLSAIFTT |
| 156 | MLPAPKNLVVSRVTEDSARLSWTAPDAAFDSFAIEYFENLGIGEA |
| | IVLTVPGSERSYDLTGLKPGTEYVVNIYGVKGGWLSSPLSAIFTT |
| 157 | MLPAPKNLVVSRVTEDSARLSWTAPDAAFDSFAIEYYEYVGNGEA |
| | IVLTVPGSERSYDLTGLKPGTEYQVGIYGVKGGYYSRPLSAIFTT |
| 158 | MLPAPKNLVVSRVTEDSARLSWTAPDAAFDSFIIDYLELDDYGEA |
| | IVLTVPGSERSYDLTGLKPGTEYPVYIYGVKGGLPSTPLSAIFIT |
| 159 | MLPAPKNLVVSRVTEDSARLSWTAPDAAFDSFLIPYAETSPSGEA |
| | IVLTVPGSERSYDLTGLKPGTEYSVLIHGVKGGRNSDPLSAIFTT |
| 160 | MLPAPKNLVVSRVTEDSARLSWTAPDAAFDSFNIAYGEWRQHGEA |
| | IVLTVPGSERSYDLTGLKPGTEYDVFIDGVKGGNLSWPLSAIFTT |
| 161 | MLPAPKNLVVSRVTEDSARLSWTAPDAAFDSFTIRYWEELPTGEA |
| | IVLTVPGSERSYDLTGLKPGTEYTVEIFGVKGGYLSRPLSAISTT |
| 162 | MLPAPKNLVVSRVTEDSARLSWTAPDAAFDSFHIAYEEATTYGEA |
| | IFLRVPGSERSYDLTGLKPGTEYDVWIEGVKGGTISGPLSAIFTT |
| 163 | MLPAPKNLVVSRVTEDSARLSWTAPDAAFDSFPIAYAEPRPDGEA |
| | IVLTVPGSERSYDLTGLKPGTEYFVDIFGVKGGILSRPLSAIFTT |
| 164 | MLPAPKNLVVSRVTEDSARLSWTAPDAAFDSFLIPYAETSPSGEA |
| | IVLTVPGSERSYDLTGLKPGTEYSVLIHGVKGGHLSDPLSAIFTT |
| 165 | MLPARKNLVVSRVTEDSARLSWTAPDAAFDSFFIPYAEPSPTGEA |
| | IVLTVPGSERSYDLTGLKPGTEYSVLIHGVKGGHLSDPLSAISTT |
| 166 | MLPAPKNLVVSRVTEDSARLSWTAPDAAFDSFTILYNEIQNVGEA |
| | IVLTVPGSERSYDLTGLKPGTEYDVWIEGVKGGELSWPLSAIFTT |
| 167 | MLPAPKNLVVSRVTEDSARLSWTAPDAAFDSFFIGYLEPQPPGEA |
| | IVLTVPGSERSYDLTGLKPGTEYNVTIQGVKGGTPSEPLSAIFTT |
| 168 | MLPAPKNLVVSRVTEDSARLSWTTPDAAFDSFFIGYLEPYPPGEA |
| | IVLTVPGSERSYDLTGLKPGTEYVVSIQGVKGGKPSDPLSAIFTT |
| 169 | MLPAPKNLVVSRVTEDSARLSWTAPDAAFDSFFIGYLEPQPPGEA |
| | IVLTVPGSERSYDLTGLKPGTEYNVTIQGVKGGFPSVPLSAIFTT |
| 170 | MLPAPKNLVVSRVTEDSARLSWTAPDAAFDSFFIGYPEYPATGEA |
| | IVLTVPGSERSYDLTGLKPGTEYFVDINGVKGGSLSYPLSAIFTT |
| 171 | MLPAPKNLVVSRVTEDSARLSWTAPDAAFDSFAIRYLEWWDVGEA |
| | IVLTVPGSERSYDLTGLKPGTEYLVEIKGVKGGKFSYPLSAIFTT |
| 172 | MLPAPKNLVVSRVTEDSARLSWTAPDAAFDSFPIEYDEWWALGEA |
| | ITLIVPASERSYDLTGLKPGTEYVVKIHGVKGGQRSYPLIAFFTT |
| 173 | MLPAPKNLVVSRVTEDSARLSWTAPDAAFDSFTIHYRELYVQAIV |
| | LTVPGSERSYDLTGLKPGTEYLVMIPGVKGGPTSVPLSAIFTT |
| 174 | MLPAPKNLVVSRVTEDSARLSWTAPDAAFDSFFIGYLEPQPPGEA |
| | IVLTVPGSERSYDLTGLKPGTEYKVVIQGVKGGTPSEPLSAIFTT |
| 175 | MLPAPKNLVVSRVTEDSARLSWTAPDAAFDSFFIGYLEPQPPGEA |
| | IVLTVPGSERSYDLTGLKPGTEYSVVIQGVKGGFPSDPLSAIFTT |
| 176 | MLPAPKNLVVSRVTEDSARLSWTAPDAAFDSFPIAYAEPRPDGEA |
| | IVLTVPGSERSYDLTGLKPGTEYSVGIHGVKGGHDSSPLSAIFTT |
| 177 | MLPAPKNLVVSRVTEDSARLSWTAPDAAFDSFFIGYLEPQPPGEA |
| | IVLTVPGSERSYDLTGLKPGTEYNVTIQGVKGGRASGPLSAIFTT |
| 178 | MLPAPKNLVVSRVTEDSARLSWTAPDAAFDSFDIAYAEPIPRGEA |
| | IVLTVPGSERSYDLTGLKPGTEYSVLIHGVKGGRRSVPLSAIFTT |
| 179 | MLPAPKNLVVSRVTEDSARLSWTAPDAAFDSFYIAYAEPRPDGEA |
| | IVLTVPGSERSYDLTGLKPGTEYPVPIPGVKGGPGSSPLSAIFTT |
| 180 | MLPAPKNLVVSRVTEDSARLSWTAPDAAFDSFEISYYEMRGYGEA |
| | IVLTVPGSERSYDLTGLKPGTEYSVLIHGVEGGDYSSPLSAISTT |
| 181 | MLPAPKNLVVSHVTEDSARLSWTAPDAAFDSFPIAYAEPRPDGEA |
| | IVLTVPGSERSYDLTGLKPGTEYSVLIHGVKGGLLSSPLSAIFTT |
| 182 | MLPAPKNLVVSRVTEDSARLSWTAPDAAFDSFFIGYLEPYPPGEA |
| | IVLTVPGSERSYDLTGLKPGTEYVVSIQGVKGGTPSQPLSAIFTT |
| 183 | MLPAPKNLVVSRVTEDSARLSWTAPDAAFDSFLIPYAETSPSGEA |
| | IVLTVPGSERSYDLTGLKPGTEYSVLIHGVKGGRPSNPLVAAFTT |
| 184 | MLPAPKNLVVSRITEDSARLSWTAPDAAFDSFGIGYYEHKRFGEA |
| | IQLSVPGSERSYDLTGLKPGTEYEVDIEGVKGGVLSWPLFAEFTT |
| 185 | MLPAPKNLVVSRVTEDSARLSWTAPDAAFDSFEIDYDELAIYGEA |
| | IVLTVPGSERSYDLTGLKPGTEYGVMIIGVKGGLPSDPLSAIFTT |
| 186 | MLPAPKNLVVSRVTEDSARLSWTAPDAAFDSFFIGYLESAEAIVL |
| | TVPGSERSYDLTGLKPGTEYLVTIQGVKGGIASDPLSAIFTT |
| 187 | MLPAPKNLVVSRVTEDSARLSWTAPDAAFDSFVIEYFEFVGYGEA |
| | IVLTVPGSERSYDLTGLKPGTEYSVGIYGVKGGKLSPPLSAIFTT |
| 188 | MLPAPKNLVVSRVTEDSARLSWTAPDAAFDSFLIPYAETSPSGEA |
| | IVLTVPGSERSYDLTGLKPGTEYSVLIHGVKGGKLSLPLSAIFTT |
| 189 | MLPAPKNLVVSRVTEDSARLSWTAPDAAFDSFHEWVYFGEAIVLT |
| | VPGSERSYDLTGLKPGTEYFVDIWGVKGGTVSKPLSAIFTT |
| 190 | MLPAPKNLVVSRVTEDSARLSWTAPDAAFDSFFIGYPEYPATGEA |
| | ITLFVPGSERSYDLTGLKPGTEYNVVIQGVKGGRPSNPLVVAFTT |
| 191 | MLPAPENLVVSRVTEDSARLSWTAPDAAFDSFEITYEENWRRGEA |
| | IVLTVPGSERSYDLTGPKPGTEYIVIIQGVKGGAESWPLSAIFTT |
| 192 | MLPAPKNLVVSRVTEDSARLSWTALDAAFDSFFIGYLEPQPPGEA |
| | IVLTVPGSERSYDLTGLKPGTEYNVTIQGVKGGFPSMPLSAIFTT |
| 193 | MLPAPKNLVVSRVTEDSARLSWTAPDAAFDSFTIWYAEAVGNGEA |
| | IVLTVPGSERSYDLTGLKPGTEYWVDIWGVKGGEFSSPLSAIFTT |
| 194 | MLPAPKNLVVSRVTEDSARLSWTAPDAAFDSFEIDYDELAIYGEA |
| | IVLTVPGSERSYDLTGLKPGTEYRVFIYGVKGGWTSWPLSTIFTT |
| 195 | MLPAPKNLVVSRVTEDSARLSWTAPDAAFDSFPIEYDEIPFWGEA |
| | IVLTVPGSERSYDLTGLKPGTEYRVWIHGVKGGNSSWPLSAIFTT |
| 196 | MLPAPKNLVVSRVTEDSARLSWTAPDAAFDSFNIHYVEWWVLGEA |
| | IVLTVPGSERSYDLTGLKPGTEYPVYIYGVKGGPKSIPLSAIFTT |
| 197 | MLPAPKNLVVSRVTEDSARLSWTAPDAAFDSFKIDYLEINDNGEA |
| | IVLTVPGSERSYDLTGLKPGTEYPVYIWGVKGGYPSSPLSAIFTT |
| 198 | MLPAPKNLVVSRVTEDSARLSWTAPDAAFDSFLIAYNEDRKFGEA |
| | IVLTVPGSERSYDLTGLKPGTEYDVWIEGVKGGSLSFPLSAIFTT |
| 199 | MLPAPKNLVVSRVTEDSARLSWTAPDAAFDSFGIRYFEWWDLGEA |
| | IVLTVPGSERSYDPTGLKPGTEYNVTIQGVKGGFPSMPLSAIFTT |
| 200 | MLPAPKNLVVSRVTEDSARLSWTAPDAAFDSFYIEYYEWMHTGEA |
| | IVLTVPGSERSYDLTGLKPGTEYSVYIYGVKGGYPSSPLSAIFTT |
| 201 | MLPAPKNLVVSRVTEDSARLSWTAPDAAFDSFNIDYWETWVIGEA |
| | IVLTVPGSERSYDLTGLKPGTEYEVIIPGVKGGTISPPLSAIFTT |
| 202 | MLPAPKNLVVSRVTEDSARLSWTAPDAAFDSFYIDYLELTYSGEA |
| | IVLTVPGSERSYDLTGLKPGTEYYVYIYGVKGGYPSSPLSAIFTT |
| 203 | MLPAPKNLVVSRVTEDSARLSWTAPDAALDSFRIEYYESYGHGEA |
| | IVLTVPGSERSYDLTGLKPGTEYDVGIYGVKGGYYSRPLSAIFTT |
| 204 | MLPAPKNLVVSRVTEDSARLPWTAPDAAFDSFWISYYESVGYGEA |
| | IVLTVPGSERSYDLTGLKPGTEYYVDISGVKGGVYSLPLSAIFTT |
| 205 | MLPAPKNLVVSRVTEDSARLSWTAPDAAFDSFDIDYDEPAWNGEA |
| | IVLTVPGSERSYDLTGLKPGTEYRVFIYGVKGGNTSWPLSAIFTT |
| 206 | MLPAPKNLVVSRVTEDSARLSWTAPDAAFDSFDIEYDELWKNGEA |
| | IVLTVPGSERSYDLTGLKPGTEYRVFIYGVKGGYGSFPLSAIFTT |
| 207 | MLPAPKNLVVSRVTEDSARLSWTAPDAAFDSFFIGYLEPQPPGEA |
| | IVLTVPGSERSYDLTGLKPGTEYNVTIQGVKGGTPSEPLSAISTT |
| 208 | MLPAPKNLVVSRVTEDSARLSWTAPDAAFDSFGIVYREPYVGGEA |
| | IVLTVPGSERSYDLTGLKPGTEYGVPIPGVKGGYDSGPLSAIFTT |
| 209 | MLPAPKNLVVSRVTEDSARLSWTAPDAAFDSFTIPYIEYVWWGEA |
| | IVLTVQGSERSYDLTGLKPGTEYPVTIGGVKGGSRSHPLHAHFTT |
| 210 | MLPAPKNLVVSRVTEDSARLSWTAPDAAFDSFSIVYGERFVNGEA |
| | IVLTVPGSERSYDLTGLKPGTEYHVYIDGVKGGDLSWPLSAIFTT |
| 211 | MLPAPKNLVVSRVTEDSARLSWTAPDAAFDSFWINYYEAQPDGEA |
| | IVLTVPGSERSYDLTGLKPGTEYDVEIAGVKGGTASLPLSAIFTT |
| 212 | MLPAPKNLVVSRVTEDSARLSWTAPDAAFDSFIIEYWEQIGVGEA |
| | IVLTVPGSERSYDLTGLKPGTEYWVGIYGVKGGLLSSPLSAIFTT |
| 213 | MLPAPKNLVVSRVTEDSARLSWTAPDAAFDSFWIYYWEIERAGEA |
| | IRLDVPGSERSYDLTGLKPGTEYRVDIWGVKGGPTSGPLRATFTT |
| 214 | MLPAPKNLVVSRVTEDSARLSWTAPDAAFDSFAIPYGERQELGEA |
| | IVLTVPGSERSYDLTGLKPGTEYFVVIQGVKGGQPSYPLSAIFTT |
| 215 | MLPAPKNLVVSRVTEDSARLSWTAPDAAFDSFLIPYAETSPTGEA |
| | IVLTVPGSERSYDLTGLKPGTEYSVLIHGVKGGYPSSPLSAIFTT |
| 216 | MLPAPKNLVVSRVTEDSARLSWTAPDAAFDSFPIAYAEPTPSGEA |
| | IVLTVPGSERSYDLTGLKPGTEYSVLIHGVKGGGLSLPLSAIFTT |
| 217 | MLPAPKNLVVSRVTEDSARLSWTAPDAAFDSFIIEYWEWYFAGEA |
| | IVLTVPGSERSYDLTGLKPGTEYTVWITGVKGGTWSEPLSAIFTT |
| 218 | MLPAPKNLVVSRVTEDSARLSWTAPDAAFDSFTILYYEMVGEGEA |
| | IVLTVPGSERSYDLTGPKPGTEYWVDIYGVKGGGWSRPLSAIFTT |
| 219 | MLPAPKNLVVSRVTEDSARLSWTAPDAAFDSFHIDYLELTYAGEA |
| | IVLTVPGSERSYDLTGLKPGTEYYVTIYGVKGGYPSSPLSAIFTT |
| 220 | MLPAPKNLVVSRVTEDSARLSWTAPDAAFDSFHIIYEEDGTEGEA |
| | IYLRVPGSERSYDLTGLKPGTEYEVDIEGVKGGVLSWPLFAEFTT |
| 221 | MLPAPKNLVVSRVTEDSARLSWTAPDAAFDSFHISYQEVVAEGEA |
| | IYLRVPGSERSYDLTGLKPGTEYYVLIHGVKGGYESKPLDASFTT |
| 222 | MLPAPKNLVVSRVTEDSARLSWTAPDAAFDSFYIEYFEWTGSGEA |
| | IVLTVPGSERSYDLTGLKPGTEYNVAIYGVKGGAVSYPLSAIFTT |
| 223 | MLPAPKNLVVSRVTEDSARLSWTAPDAAFDSFTIWYAEALGDGEA |
| | IVLTVPGSERSYDLTGLKPGTEYFVDIPGVKGGTRSSPLSAISTT |
| 224 | MLLAPKNLVVSRVTEDSARLSWTAPDAAFDSFRYLEQGLYGEAIV |
| | LTVPGSERSYDLTGLKPGTEYWVEIIGVKGGEYSTPLSAIFTT |
| 225 | MLPAPKNLVVSRVTEDSARLSWTAPDAAFDSFKIEYFEYVGYGEA |
| | IVLTVPGSERSYDLTGLKPGTEYYVAIYGVKGGWYSRPLSAIFTT |
| 226 | MLPAPKNLVVSRVTEDSARLSWTAPDAAFDSFHIIYEEVLTEGEA |
| | IYLRVPGSERSYDLTGLKPGTEYGVTIKGVKGGAYSIPLIATFTT |
| 227 | MLPAPKNLVVSRVTEDSARLSWTAPDAAFDSFRIRYLEWWNIGEA |
| | IVLTVPGSERSYDLTGLKPGTEYHVDIWGVKGGYSSYPLSAIFTT |
| 228 | MLPAPKNLVVSRVTEDSARLSWTAPDAAFDSFEIYYVEWSEAGEA |
| | IVLTVPGSERSYDLTGLKPGTEYRVEIRGVKGGSWSSPLSAIFTT |
| 229 | MLPAPKNLVVSRVTEDSARLSWTAPDAAFDSFSIHYDEDWRRGEA |
| | IVLTVPGSERSYDLTGLKPGTEYLVEIPGVKGGKASYPLSAIFTT |
| 230 | MLPAPKNLVVSRVTEDSARLSWTAPDAAFDSFQIRYPKRWISGEA |
| | IVLTVPGSERSYDLTGLKPGTEYEVVIRGVKGGEYSWPLSAIFTT |
| 231 | MLPAPKNLVVSRVTEDSARLSWTAPDAAFDSFEIPYIETVALGEA |
| | IVLTVPGSERSYDLTGLKPGTEYYVEIYGVKGGSYSYPLSAISTT |
| 232 | MLPAPKNLVVSRVTEDSARLSWTAPDAAFDSFTIAYDETLNLGEA |
| | IVLTVPGSERSYDLTGLKPGTEYIVGIFGVKGGTHSWPLSAIFTT |
| 233 | MLPAPKNLVVSRVTEDSARLSWTAPDAAFDSFYIVYAEPIPNGEA |
| | IVLTVPGSERSYDLTGLKPGTEYSVLIHGVKGGRNSDPLSAIFTT |
| 234 | MLPAPKNLVVSRVTEDSARLSWTAPDAAFDSFYITYWETWDYGEA |
| | IVLTVPGSERSYDLTGLKPGTEYKVPITGVKGGGPSVPLSAIFTT |
| 235 | MLPAPKNLVVSRVTEDSARLSWTAPDAAFDSFSINYREWWSDGEA |
| | IYLPVPGSERSYDLTGLKPGTEYAVYIQGVKGGSRSFPLHAWFTT |
| 236 | MLPAPKNLVVSRVTEDSARLSWTAPDAAFDSFWIEYYEELGSGEA |
| | IVLTVPGSERSYDLTGLKPGTEYRVYIYGVKGGYPSSPLSAIFTT |
| 237 | MLPAPKNLVVSRVTEDSARLSWTAPDAAFDSFTILYGEMGTTGEA |
| | IVLTVPGSERSYDLTGLKPGTEYDVFIEGVKGGELSWPLSAIFTT |
| 238 | MLPAPKNLVVSRVTEDSARLSWTAPDAAFDSFKIFYQEFGGEAIV |
| | LTVPGSERSYDLTGLKPGTEYWVDIYGVKGGYTSSPLSAIFTT |
| 239 | MLPAPKNLVVSRVTEDSARLSWTAPDAAFDSFAITYYEGRWRGEA |
| | IVLTVPGSERSYDLTGLKPGTEYGVPIRGVKGGTGSLPLSAIFTT |
| 240 | MLPAPKNLVVSRVTEDSARLSWTAPDAAFDSFRIKYLEWWLGGEA |
| | IVLTVPGSERSYDLTGLKPGTEYWVDIQGVKGGVLSWPLSAIFTT |
| 241 | MLPAPKNLVVSRVTEDSARLSWTAPDAAFDSFNIYYYEWFVSGEA |
| | IVLTVPGSERSYDLTGLKPGTEYFVDIDGVKGGYRSRPLSAIFTT |
| 242 | MLPAPKNLVVSRVTEDSARLSWTAPDAAFDSFPIKYLEWWSWGEA |
| | IVLTVPGSERSYDLTGLKPGTEYRVPISGVKGGGMSGPLSAIFIT |
| 243 | MLPAPKNLVVSRVTEDSARLSWTAPDAAFDSFAIPYYEWVNHGEA |
| | IVLTVPGSERSYDLTGLKPGTEYPVGIDGVKGGGPSWPLSAIFIT |
| 244 | MLPAPKNLVVSRVTEDSARLSWTAPDAAFDSFHIDYSEFHLRGEA |
| | IVLTVPGSERSYDLTGLKPGTEYLGIFGVKGGEQSGPLSAIFTT |
| 245 | MLPAPKNLVVSRITEDSARLSWTAPDAAFDSFGIAYNEGDHYGEA |
| | IVLTVPGSERSYDLTGLKPGTEYSVWIEGVKGGNLSYPLSAIFTT |
| 246 | MLPAPKNLVVSRVTEDSARLSWTAPDAAFDSFTIAYNEQNHYGEA |
| | IVLTVPGSERSYDLTGLKPGTEYGVWIEGVKGGTLSWPLSAIFIT |
| 247 | MLPAPKNLVVSRVTEDSARLSWTAPDAAFDSFYIEWTYKGEAIVL |
| | TVPGSERSYDLTGLKPGTEYFVGIPGVKGGKSSYPLSAIFTTNPK |
| | GDTP |
| 248 | MGSLPAPKNLVVSRVTEDSARLSWTAPDAAFDSFAIPYAEPSPTG |
| | EAIVLTVPGSERSYDLTGLKPGTEYPVWIQGVKGGSPSAPLSAEF |
| | TT |
| 249 | MLPAPKNLVVSRVTEDSARLSWTAPDAAFDSFSIDYFESVGFGEA |
| | IVLTVPGSERSYDLTGLKPGTEYDVQITGVKGGPHSLPLSAIFTT |
| 250 | MLPAPKNLVVSRVTEDSARLSWTAPDAAFDSFFIGYLEPYPPGEA |
| | IVLTVPGSERSYDLTGLKPGTEYAVEIAGVKGGLLSSPLSAISTT |
| 251 | MLPAPKNLVVSRVTEDSARLSWTAPDAAFDSFFIGYLEPQPPGEA |
| | IVLTVPGSERSYDLTGLKPGTEYNVTIQGVKGGFPSMPLSAIVTT |
| 252 | MLPAPKNLVVSRVTEDSARLSWTAPDAAFDSFDIGYTEYGGYGEA |
| | IVLTVPGSERSYDLTGLKPGTEYWVLIQGVKGGGSSVPLSAIFTT |
| 253 | MLPAPKNLVVSRVTEDSARLSWTAPDAAFDSFTIEYWETIGGGEA |
| | IVLTVPGSERSYDLTGLKPGTEYYVGIYGVKGGWWSRPLSAIFIT |
| 254 | MLPAPKNLVVSRVTEDSARLSWTAPDAAFDSFFIPYAEPSPTGEA |
| | IVLTVPGSERSYDLTGLKPGTEYSVLIHGVKGGHLSDPLSAISTT |
| 255 | MLPAPKNLVVSRVTEDSARLSWTAPDAAFDSFDIEYYELIGRGEA |
| | IVLTVPGSERSYDLTGLKPGTEYWVGIYGVKGGWLSRPLSAIFTT |
| 256 | MLPAPKNLVVSRVTEDSARLSWTAPDAAFDSFHIVYHEPRPSGEA |
| | IVLTVPGSERSYDLTGLKPGTEYEVGIVGVKGGDLSVPLSAIFTT |
| 257 | MLPAPKNLVVSRVTEDSARLSWTAPDAAFDSFHIVYHEPRPSGEA |
| | IVLTVPGSERSYDLTGLKPGTEYEVGIVSVKGGDLSVPLSAIFTT |
| 258 | MLPAPKNLVVSRVTEDSARLSWTAPDAAFDSFFIPYAEPSPTGEA |
| | IVLTVPGSERSYDLTGLKPGTEYDVWIEGVKGGVLSWPLSAIFTT |
| 259 | MLPAPKNLVVSRVTEDSARLSWTAPDAAFDSFTIEYFEFVDAGEA |
| | IVLTVPGSERSYDLTGLKPGTEYWVEIWGVKGGSWSKPLSAIFTT |
| 260 | MLPAPKNLVVSRVTEDSARLSWTAPDAAFDSFNISYYEYFVHGEA |
| | IVLTVPGSERSYDLTGLKPGTEYYVIDGVKGGDPSEPLSAIFTT |
| 261 | MLPAPKNLVVSRVTEDSARLSWTAPDAAFDSFTIVYGEWGVPGEA |
| | IVLTVPGSERSYDLTGLKPGTEYDVWIEGVKGGDLSWPLSAIVTT |
| 262 | MLPAPKNLVVSRVTEDSARLSWTAPDAAFDSFAIEYFEYTGEGEA |
| | IVLTVPGSERSYDLTGLKPGTEYYVGIYGVKGGYLSRPLSAIFTT |
| 263 | MLPAPKNLVVSRVTEDSARLSWTAPDAAFDSFFIGYLEPQPPGEA |
| | IVLTVPGSERSYDLTGLKPGTEYNVTIQGVKGGFPSMPLSAISTT |
| 264 | MLPAPKNLVVSRVTEDSARLSWTAPDAAFDSFRIKYQEWWVEGEA |
| | IVLTVPGSERSYDLTGLKPGTEYVVQIAGVKGGLSSYPLSAIFIT |
| 265 | MLPAPKNLVVSRVTEDSARLSWTAPDAAFDSFWIYYIETSHQGEA |
| | IVLTVPGSERSYDLTGLKPGTEYFVLIKGVKGGYDSVPLSAIFTT |
| 266 | MLPAPKNLVVSRVTEDSARLSWTAPDAAFDSFMIRYQEGTRWGEA |
| | IVLTVPGSERSYDLTGLKPGTEYIVMIAGVKGGQISLPLSAIFTT |
| 267 | MLPAPKNLVVSRVTEDSARLSWTAPDAAFDSFPIVYSEIHVIGEA |
| | IVLTVPGSERSYDLTGLKPGTEYDVWIEGVKGGHLSEPLSAIFTT |
| 268 | MLPAPKNLVVSRVTEDSARLSWTAPDAAFDSFTIVYGEAGAFGEA |
| | IVLTVPGSERSYDLTGLKPGTEYDVLIEGVKGGNLSWPLSAIFTT |
| 269 | MLPAPKNLVVSRVTEDSARLSWTAPDAAFDSFHINYAEVYTKGEA |
| | ILLTVPGSERSYDLTGLKPGTEYEVYIPGVKGGPFSRPLNAQFTT |
| 270 | MLPAPKNLVVSRVTEDSARLSWTAPDAAFDSFLIRYQEWQRWGEA |
| | IVLTVPGSERSYDLTGLKPGTEYTVHIAGVKGGMLSLPLSAIFTT |
| 271 | MLPAPKNLVVSRVTEDSARLSWTAPDAAFDSFYIPYAETRDDGEA |
| | IVLTVPGSERSYDLTGLKPGTEYSVLIHGVKGGDLSSPLSAIFTT |
| 272 | MLPAPKNLVVSRVTEDSARLSWTAPDAAFDSFGIPYAESTPTGEA |
| | IVLTVPGSERSYDLTGLKPGTEYSVLIHGVKGGHLSDPLSAIFTT |
| 273 | MLPAPKNLVVSRVTEDSARLSWTAPDAAFDSFTIFKDGEAIVLTV |
| | PGSERSYDLTGLKPGTEYYVYIYGVKGGYPSKPLSAIFTT |
| 274 | MLPAPKNLVVSRVTEDSVRLSWTAPDAAFDSFAISYEEWWVHGEA |
| | IVLTVPGSERSYDLTGLKPGTEYSVVIPGVKGGLYSWTLSAISTT |
| 275 | MLPAPKNLVVSRVTEDSARLSWTAPDAAFDSFLIAYAEVTLHGEA |
| | IVLTVPGSERSYDLTGLKPGTEYSVLIHGVKGGRNSDPLSAIFTT |
| 276 | MLPAPKNLVVSRVTEDSARLSWTAPDAAFDSFRIDYLELTSLGEA |
| | IVLTVPGSERSYDLTGLKPGTEYPVPILGVKGGLSSWPLSAIFTT |
| 277 | MLPAPKNLVVSRVTEDSARLSWTAPDAAFDSFWINYYEGIGEGEA |
| | IVLTVPGSERSYDLTGLKPGTEYYVDISGVKGGSYSLPLSAIFTT |
| 278 | MLPAPKNLVVSRVTEDSARLSWTAPDAAFDSFPIAYAEPRPDGEA |
| | IVLTVPGSERSYDLTGLKPGTEYSVLIHGVKGGHLSDPLSAIFTT |
| 279 | MLPAPKNLVVSRVTEDSARLSWTAPDAAFDSFSIEYYESVGLGEA |
| | IVLTVPGSERSYDLTGLKPGTEYDVSIYGVKGGYLSRPLSAIFIT |
| 280 | MLPAPKNLVVRXVTEDSARLSWTAPDAAFDSFEIEYDEPYRGGEA |
| | IVLTVPGSERSYDLTSLKPGTEYPVSIGGVKGGITSDPLSAIFTT |
| 281 | MLPAPKNLVVSRVTEDSARLSWTAPDAAFDSFYIDYDEIHDWGEA |
| | IVLTVPGSERSYDLTGLKPGTEYAVQIGGVKGGSFSWILSAIFTT |
| 282 | MLPAPKNLVVSRVTEDSARLSWTAPDAAFDSFAIVYHEPRPDGEA |
| | IVLTVPGSERSYDLTGLKPGTEYEVVILGVKGGVHSYPLSAIFTT |
| 283 | MLPAPKNLVVSRVTEDSARLSWTAPDAAFDSFPIAYAEPRPDGEA |
| | IVLTVPGSERSYDLTGLKPGTEYSVLIHGVKGGLLSSPLSAIFTT |
| 284 | MLPAPKNLVVSRVTEDSARLSWTAPDAAFDSFFIGYLEPQPPGEA |
| | IVLTVPGSERSYDLTGLKPGTEYNVTIQGVKGGFPSMPLSAIFTT |
| 285 | MLPAPKNLVVSRVTEDSARLSWTAPDAAFDSFLIPYAETSPSGEA |
| | IVLTVPGSERSYDLTGLKPGTEYSVLIHGVKGGDYSSPLSAIFIT |
| 286 | MLPAPKNLVVSRVTEDSARLSWTAPDAAFDSFNIYYPEFPVRGEA |
| | IVLTVPGSERSYDLTGLKPGTEYVVSIWGVKGGTQSWPLSAIFTT |
| 287 | MLPAPKNLVVSRVTEDSARLSWTAPDAAFDSFTIEYHESGPVGEA |
| | IVLTVPGSERSYDLTGLKPGTEYMVWIFGVKGGFVSRPLSAIFTT |
| 288 | MLPAPKNLVVSRVTEDSARLSWTAPDAAFDSFLIPYAETSPSGEA |
| | IVLTVPGSERSYDLTGLKPGTEYSVLIHGVKGGDYSSPLSAISTT |
| 289 | MLPAPKNLVVSRVTEDSARLSWTAPDAAFDSFAIPYYEDTNDGEA |
| | IVLTVPGSERSYDLTGLKPGTEYWVSIQGVKGGTVSGPLSAIFTT |
| 290 | MLPAPKNLVVSRVTEDSARLSWTAPDAAFDSFFYLEQAWGGEAIV |
| | LTVPGSERSYDLTGLKPGTEYWVEITGVKGGYASSPLSAIFTT |
| 291 | MLPAPKNLVVSRVTEDSARLSWTAPDAAFDSFHIEYEEPETEGEA |
| | IYLHVPGSERSYDLTGLKPGTEYKVLIRGVKGGSYSIPLQAPFTT |
| 292 | MLPAPKNLVVSRVTEDSARLSWTAPDAAFDSFDIAYWELTPSGEA |
| | IELLVPGSERSYDLTGLKPGTEYRVDIIGVKGGFISEPLGATFTT |
| 293 | MLPAPKNLVVSRVTEDSARLSWTAPDAAFDSFTIEYWEFTGSGEA |
| | IVLTVPGSERSYDLTGLKPGTEYDVSIYGVKGGWLSYPLSAIFTT |
| 294 | MLPAPKNLVVSRVTEDSARLSWTAPDAAFDSFSIIYSEWNVTGEA |
| | IVLTVPGSERSYDLTGLKPGTEYDVWIEGVKGGGMSKPLSAISTT |
| 295 | MLPAPKNLVVSRVTEDSARLSWTAPDAAFDSFFIGYLEPIPSGEA |
| | IVLTVPGSERSYDLTGLKPGTEYPVVIQGVKGGHPSQPLSAIFIT |
| 296 | MLPAPKNLVVSRVTEDSARLSWTAPDAAFDSFFIGYLEPQPPGEA |
| | IILTVPGSERSYDLTGLKPGTEYNVTIQGVKGGFPSMPLSAIFTT |
| 297 | MLPAPKNLVVSRVTEDSARLSWTAPDAAFDSFLIPYAETSPSGEA |
| | ITLFVPGSERSYDLTGLKPGTEYNVVIQGVKGGRPSNPLVAASTT |
| 298 | MLPAPKNLVVSRVTEDSARLSWTAPDAAFDSFPIAYAEPRPDGEA |
| | IVLTVPGSERSYDLTGLKPGTEYSVLIHGVKGGLLSSPLSAISTT |
| 299 | MLPAPKNLVVSRVTEDSARLSWTAPDAAFDSFLIEYWESVGYGEA |
| | IVLTVPGSERSYDLTGLKPGTEYWVGIYGVKGGYYSRPLSAIFTT |
| 300 | MLPAPKNLVVSRVTEDSARLSWTAPDAAFDSFFIGYLEPQPPGEA |
| | IVLTVPGSERSYDLTGLKPGTEYNVTIHGVKGGTPSMPLSAIFTT |
| 301 | MLPAPKNLVVSRVTEDSARLSWTAPDAAFDSFEIEYDEPYRGGEA |
| | IVLTVPGSERSYDLTSLKPGTEYPVSIGGVKGGITSDPLSAIFTT |
| 302 | MLPAPKNLVVSRVTEDSARLSWTAPDAAFDSFDIYYPEYYDRGEA |
| | IVLTVPGSERSYDLTGLKPGTEYTVYIDGVKGGGGSGPLSAIFTT |
| 303 | MLPAPKNLVVSRVTEDSARLSWTAPDAAFDSFFIAYFEFANPGEA |
| | IVLTVPGSERSYDLTGLKPGTEYKVVIQGVKGGTPSEPLSAIFTT |
| 304 | MLPAPKNLVVSRVTEDSARLSWTAPDAAFDSFIITYWEHVGDGEA |
| | IVLTVPGSERSYDLTGLKPGTEYFVEIYGVKGGYLSKPLSAIFTT |
| 305 | MLPAPKNLVVSRVTEDSARLSWTAPDAAFDSFEIDYDEPFVYGEA |
| | IVLTVPGSERSYDLTGLKPGTEYRVFIFGVKGGNGSWPLSAIFTT |
| 306 | MLPAPKNLVVSRVTEDSARLSWTAPDAAFDSFYIEYFETQGYGEA |
| | IVLTVPGSERSYDLTGLKPGTEYYVAIYGVKGGYLSRPLSAIFTT |
| 307 | MLPAPKNLVVSRVTEDSARLSWTAPDAAFDSFPITYSEPAHYGEA |
| | IVLTVPGSERSYDLTGLKPGTEYHVGIMGVKGGVFSSPLSAIFTT |
| 308 | MLPAPKNLVVSEVTEDSARLSWQGVARAFDSFLITYREQIFAGEV |
| | IVLTVPGSERSYDLTGLKPGTEYPVWIQGVKGGSPSAPLSAISTT |
| 309 | MLPAPKNLVVSRVTEDSARLSWTAPDAAFDSFIIDYLELDQEGEA |
| | IVLTVPGSERSYDLTGLKPGTEYAVYIFGVKGGYPSTPLSAIFTT |

Example 6. Knockdown of mRNA in muscle cells using CD71 FN3 domain-oligonucleotide conjugates. muCD71 binding FN3 domains are conjugated to siRNA oligonucleotides or antisense oligonucleotides (ASOs) using maleimide chemistry via a cysteine that is uniquely engineered into the FN3 domain. The cysteine substitutions can be one such as those provided for herein and also as provided for in U.S. Patent Application Publication No. 20150104808, which is hereby incorporated by reference in its entirety. siRNAs or ASOs are modified with standard chemical modifications and confirmed to enable knockdown of the targeted mRNA in vitro. FN3 domain-oligonucleotide conjugates are dosed intravenously in mice at doses up to 10 mg/kg oligonucleotide payload. At various time points following dosing, mice are sacrificed; skeletal muscle, heart muscle and various other tissues will be recovered and stored in RNAlater™ (Sigma Aldrich) until needed. Target gene knockdown is assessed using standard qPCR ΔΔC_{T} methods and primers specific for the target gene and a control gene. The target gene is found to be knock downed in the muscles and such knockdown is enhanced by conjugating the siRNA or ASO to the CD71 FN3 binding domain.

### Example 7. General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57 (b)(1), to relate to each and every individual publication, database entry (e.g. Genbank sequences or GeneID entries), patent application, or patent, each of which is clearly identified in compliance with 37 C.F.R. § 1.57 (b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the embodiments in addition to those described herein will become apparent to those skilled in the art from the foregoing description. Such modifications are intended to fall within the scope of the appended claims.

The foregoing written specification is considered to be sufficient to enable one skilled in the art to practice the embodiments. Various modifications of the embodiments in addition to those shown and described herein will become apparent to those skilled in the art from the foregoing description and fall within the scope of the appended claims.

1.-52. (canceled)

53. A method of delivering an agent of interest to a muscle cell, wherein the agent of interest is a polynucleotide, the method comprising contacting the muscle cell with the polynucleotide conjugated to a polypeptide comprising an amino acid sequence that is at least 90% identical to an amino acid sequence selected from the group consisting of SEQ ID NOs: 33-39, 41-62, 81-136, 138-186, 188-261, and 263-309, or at least 91% identical to an amino acid sequence selected from the group consisting of SEQ ID NOs: 40, 137, 187, and 262, or any combination thereof.

54. The method of claim 53, wherein the polynucleotide is a double-stranded short interfering ribonucleic acid (siRNA) molecule, a sense strand or an antisense strand thereof.

55. The method of claim 53, wherein the polynucleotide is an antisense molecule.

56. The method of claim 53, wherein the polypeptide comprises a methionine at the N-terminus of the polypeptide.

57. The method of claim 53, wherein the polypeptide comprises a cysteine substitution at residue position 54.

58. The method of claim 53, wherein the polypeptide comprises a half-life extending moiety.

59. The method of claim 58, wherein the half-life extending moiety is an albumin-binding molecule.

60. The method of claim 59, wherein the albumin-binding molecule is a polypeptide that binds albumin or an albumin variant.

61. The method of claim 58, wherein the half-life extending moiety is a polyethylene glycol, albumin, an albumin variant, or a portion of an Fc region of an immunoglobulin.

62. The method of claim 53, wherein the polypeptide comprises an amino acid sequence that is at least 91% identical to the amino acid sequence of SEQ ID NO: 146.

63. The method of claim 62, wherein the polypeptide comprises an amino acid sequence that is at least 95% identical to the amino acid sequence of SEQ ID NO: 146.

64. The method of claim 53, wherein the polypeptide comprises the amino acid sequence of SEQ ID NO: 146.

65. The method of claim 53, wherein the polypeptide comprises an amino acid sequence at least 90% identical to the amino acid sequence of SEQ ID NO: 179.

66. The method of claim 53, wherein the polypeptide comprises an amino acid sequence at least 90% identical to the amino acid sequence of SEQ ID NO: 96, 115, 152, 174, 175, 177, 192, 207, 263, 284, 296, or 300.

67. The method of claim 53, wherein the polypeptide comprises the amino acid sequence of SEQ ID NO: 252.

68. The method of claim 53, wherein the polypeptide comprises the amino acid sequence of SEQ ID NO: 168.

69. The method of claim 53, wherein the polypeptide comprises the amino acid sequence of SEQ ID NO: 190.

70. The method of claim 53, wherein the polypeptide comprises the amino acid sequence of SEQ ID NO: 303.

71. The method of claim 53, wherein the polypeptide comprises the amino acid sequence of SEQ ID NO: 307.

72. The method of claim 53, wherein the polypeptide binds CD71 and is linked to another polypeptide that binds CD71 or another target protein.


SEQUENCE LISTING (309 sequences)

SEQ ID NO: 1 - protein, 89 aa, Homo sapiens
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 2 - protein, 94 aa, synthetic construct
  VARIANT 75..81: note = X is any amino acid
  VARIANT 82..86: note = X is any amino acid or deleted
LPAPKNLVVSEVTEDSLRLSWTAPDAAFDSFLIQYQESEKVGEAINLTVPGSERSYDLTGLKPGTEYTVSIYGVXXXXXXXXXXXXPLSAEFTT

SEQ ID NO: 3 - protein, 89 aa, synthetic construct
  VARIANT 22..27: note = each X is Ala, Arg, Asn, Asp, Glu, Gln, Gly, His, Ile, Leu, Lys, Phe, Pro, Ser, Thr, Trp, Tyr or Val
  VARIANT 28: note = X is Phe, Ile, Leu, Val or Tyr
  VARIANT 29: note = X is Asp, Glu or Thr
  VARIANT 75..79: note = each X is Ala, Arg, Asn, Asp, Glu, Gln, Gly, His, Ile, Leu, Lys, Phe, Pro, Ser, Thr, Trp, Tyr or Val
  VARIANT 81..82: note = each X is Ala, Arg, Asn, Asp, Glu, Gln, Gly, His, Ile, Leu, Lys, Phe, Pro, Ser, Thr, Trp, Tyr or Val
LPAPKNLVVSEVTEDSLRLSWXXXXXXXXSFLIQYQESEKVGEAINLTVPGSERSYDLTGLKPGTEYTVSIYGVXXXXXSXXLSAEFTT

SEQ ID NO: 4 - protein, 89 aa, synthetic construct
LPAPKNLVVSRVTEDSARLSWTAPDAAFDSFLIQYQESEKVGEAIVLTVPGSERSYDLTGLKPGTEYTVSIYGVKGGHRSNPLSAIFTT

SEQ ID NO: 5 - protein, 97 aa, synthetic construct
  VARIANT 22..27: note = each X is A, D, E, F, G, H, I, K, L, N, P, Q, R, S, T, V, W or Y
  VARIANT 28..30: note = each X is A, D, E, F, G, H, I, K, L, N, P, Q, R, S, T, V, W, Y or deleted
  VARIANT 78..84: note = each X is A, D, E, F, G, H, I, K, L, N, P, Q, R, S, T, V, W or Y
  VARIANT 85..87: note = each X is A, D, E, F, G, H, I, K, L, N, P, Q, R, S, T, V, W, Y or deleted
LPAPKNLVVSRVTEDSARLSWXXXXXXXXXFDSFLIQYQESEKVGEAIVLTVPGSERSYDLTGLKPGTEYTVSIYGVXXXXXXXXXXSNPLSAIFTT

SEQ ID NO: 6 - protein, 96 aa, synthetic construct
  VARIANT 75..81: note = each X is A, D, E, F, G, H, I, K, L, N, P, Q, R, S, T, V, W or Y
  VARIANT 82..86: note = each X is A, D, E, F, G, H, I, K, L, N, P, Q, R, S, T, V, W, Y or deleted
LPAPKNLVVSRVTEDSARLSWTAPDAAFDSFLIQYQESEKVGEAIVLTVPGSERSYDLTGLKPGTEYTVSIYGVXXXXXXXXXXXXSNPLSAIFTT

SEQ ID NO: 7 - protein, 89 aa, synthetic construct
  VARIANT 32: note = X is A, D, E, F, G, H, I, K, L, N, P, Q, R, S, T, V, W,Y, C or M
  VARIANT 34: note = X is A, D, E, F, G, H, I, K, L, N, P, Q, R, S, T, V, W,Y, C or M
  VARIANT 36: note = X is A, D, E, F, G, H, I, K, L, N, P, Q, R, S, T, V, W,Y, C or M
  VARIANT 38..41: note = X is A, D, E, F, G, H, I, K, L, N, P, Q, R, S, T, V, W,Y, C or M
  VARIANT 68: note = X is A, D, E, F, G, H, I, K, L, N, P, Q, R, S, T, V, W,Y, C or M
  VARIANT 70: note = X is A, D, E, F, G, H, I, K, L, N, P, Q, R, S, T, V, W,Y, C or M
  VARIANT 72: note = X is A, D, E, F, G, H, I, K, L, N, P, Q, R, S, T, V, W,Y, C or M
  VARIANT 78..79: note = X is A, D, E, F, G, H, I, K, L, N, P, Q, R, S, T, V, W,Y, C or M
  VARIANT 81: note = X is A, D, E, F, G, H, I, K, L, N, P, Q, R, S, T, V, W,Y, C or M
LPAPKNLVVSRVTEDSARLSWTAPDAAFDSFXIXYXEXXXXGEAIVLTVPGSERSYDLTGLKPGTEYXVXIXGVKGGXXSXPLSAIFTT

SEQ ID NO: 8 - protein, 89 aa, synthetic construct
  VARIANT 32: note = X is A, D, E, F, G, H, I, K, L, N, P, Q, R, S, T, V, Y or W
  VARIANT 34: note = X is A, D, E, F, G, H, I, K, L, N, P, Q, R, S, T, V, Y or W
  VARIANT 36: note = X is A, D, E, F, G, H, I, K, L, N, P, Q, R, S, T, V, Y or W
  VARIANT 38..41: note = X is A, D, E, F, G, H, I, K, L, N, P, Q, R, S, T, V, Y or W
  VARIANT 46: note = X is A, D, E, F, G, H, I, K, L, N, P, Q, R, S, T, V, Y or W
  VARIANT 48: note = X is A, D, E, F, G, H, I, K, L, N, P, Q, R, S, T, V, Y or W
  VARIANT 68: note = X is A, D, E, F, G, H, I, K, L, N, P, Q, R, S, T, V, Y or W
  VARIANT 70: note = X is A, D, E, F, G, H, I, K, L, N, P, Q, R, S, T, V, Y or W
  VARIANT 72: note = X is A, D, E, F, G, H, I, K, L, N, P, Q, R, S, T, V, Y or W
  VARIANT 78..79: note = X is A, D, E, F, G, H, I, K, L, N, P, Q, R, S, T, V, Y or W
  VARIANT 81: note = X is A, D, E, F, G, H, I, K, L, N, P, Q, R, S, T, V, Y or W
  VARIANT 84: note = X is A, D, E, F, G, H, I, K, L, N, P, Q, R, S, T, V, Y or W
  VARIANT 86: note = X is A, D, E, F, G, H, I, K, L, N, P, Q, R, S, T, V, Y or W
LPAPKNLVVSRVTEDSARLSWTAPDAAFDSFXIXYXEXXXXGEAIXLXVPGSERSYDLTGLKPGTEYXVXIXGVKGGXXSXPLXAXFTT

SEQ ID NO: 9 - DNA, 19 bp, synthetic construct
  source 1..19: mol_type = other DNA
gtgacacggcggttagaac

SEQ ID NO: 10 - DNA, 20 bp, synthetic construct
  source 1..20: mol_type = other DNA
gcctttgggaagcttctaag

SEQ ID NO: 11 - DNA, 30 bp, synthetic construct
  source 1..30: mol_type = other DNA
cggcggttagaacgcggctacaattaatac

SEQ ID NO: 12 - DNA, 22 bp, synthetic construct
  source 1..22: mol_type = other DNA
catgattacgccaagctcagaa

SEQ ID NO: 13 - DNA, 385 bp, synthetic construct
  misc_difference 198..224: note = n is any base
  source 1..385: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 14 - DNA, 382 bp, synthetic construct
  misc_difference 198..221: note = n is any base
  source 1..382: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 15 - DNA, 379 bp, synthetic construct
  misc_difference 198..218: note = n is any base
  source 1..379: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 16 - DNA, 376 bp, synthetic construct
  misc_difference 198..215: note = n is any base
  source 1..376: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 17 - DNA, 131 bp, synthetic construct
  source 1..131: mol_type = other DNA
cggcggttagaacgcggctacaattaatacataaccccatccccctgttgacaattaatcatcggctcgtataatgtgtggaattgtgagcggataacaatttcacacaggaaacaggatctaccatgctg

SEQ ID NO: 18 - DNA, 30 bp, synthetic construct; identical to SEQ ID NO: 11
  source 1..30: mol_type = other DNA

SEQ ID NO: 19 - DNA, 81 bp, synthetic construct
  source 1..81: mol_type = other DNA
ccaagacagacgggcagagtcttcggtaacgcgagaaacaaccaggtttttcggcgccggcagcatggtagatcctgtttc

SEQ ID NO: 20 - DNA, 26 bp, synthetic construct
  source 1..26: mol_type = other DNA
ccgaagactctgcccgtctgtcttgg

SEQ ID NO: 21 - DNA, 45 bp, synthetic construct
  source 1..45: mol_type = other DNA
cagtggtctcacggattcctggtactggatcaggaaagagtcgaa

SEQ ID NO: 22 - DNA, 54 bp, synthetic construct
  source 1..54: mol_type = other DNA
catgcggtctcttccgaaaaagttggtgaagcgatcgtcctgaccgttccgggt

SEQ ID NO: 23 - DNA, 29 bp, synthetic construct
  source 1..29: mol_type = other DNA
ggtggtgaagatcgcagacagcgggttag

SEQ ID NO: 24 - DNA, 21 bp, synthetic construct
  source 1..21: mol_type = other DNA
cggcggttagaacgcggctac

SEQ ID NO: 25 - DNA, 61 bp, synthetic construct
  source 1..61: mol_type = other DNA
aagatcagttgcggccgctagactagaaccgctgccaccgccggtggtgaagatcgcagac

SEQ ID NO: 26 - DNA, 485 bp, synthetic construct
  misc_difference 357..392: note = n is any base
  source 1..485: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 27 - DNA, 482 bp, synthetic construct
  misc_difference 357..389: note = n is any base
  source 1..482: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 28 - DNA, 479 bp, synthetic construct
  misc_difference 357..386: note = n is any base
  source 1..479: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 29 - DNA, 476 bp, synthetic construct
  misc_difference 357..383: note = n is any base
  source 1..476: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 30 - DNA, 473 bp, synthetic construct
  misc_difference 357..380: note = n is any base
  source 1..473: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 31 - DNA, 470 bp, synthetic construct
  misc_difference 357..377: note = n is any base
  source 1..470: mol_type = other DNA
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 32 - protein, 291 aa, Homo sapiens
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 33 - protein, 90 aa, synthetic construct
MLPAPKNLVVSRVTEDSARLSWTAPDAAFDSFTIQYEELTTVGEAIYLRVPGSERSYDLTGLKPGTEYVVWIEGVKGGLRSNPLGAAFTT

SEQ ID NO: 34 - protein, 90 aa, synthetic construct
MLPAPKNLVVSRVTEDSARLSWTAPDAAFDSFAITYIEWWDVGEAIGLKVPGSERSYDLTGLKPGTEYRVHIQGVKGGNNSYPLDALFTT

SEQ ID NO: 35 - protein, 90 aa, synthetic construct
MLPAPKNLVVSRVTEDSARLSWTAPDAAFDSFEIAYFEAIWNGEAIYLTVPGSERSYDLTGLKPGTEYQVEIRGVKGGPTSRPLFAWFTT

SEQ ID NO: 36 - protein, 90 aa, synthetic construct
MLPAPKNLVVSRVTEDSARLSWTAPDAAFDSFTITYIEWWENGEAIALSVPGSERSYDLTGLKPGTEYQVGIAGVKGGYKSYPLWALFTT

SEQ ID NO: 37 - protein, 90 aa, synthetic construct
MLPAPKNLVVSRVTEDSARLSWTAPDAAFDSFHIIYTEEEKEGEAIYLRVPGSERSYDLTGLKPGTEYLVEIEGVKGGKRSVPLNASFTT

SEQ ID NO: 38 - protein, 90 aa, synthetic construct
MLPAPKNLVVSRVTEDSARLSWTAPDAAFDSFHIAYEESHTTGEAIFLRVPGSERSYDLTGLKPGTEYSVSIEGVKGGHYSPPLTAKFTT

SEQ ID NO: 39 - protein, 90 aa, synthetic construct
MLPAPKNLVVSRVTEDSARLSWTAPDAAFDSFDIDYREWWTLGEAIVLTVPGSERSYDLTGLKPGTEYYVNIQGVKGGLRSYPLSAIFTT

SEQ ID NO: 40 - protein, 90 aa, synthetic construct
MLPAPKNLVVSRVTEDSARLSWTAPDAAFDSFVIEYWEYVGHGEAIVLTVPGSERSYDLTGLKPGTEYSVGIYGVKGGSLSRPLSAIFTT

SEQ ID NO: 41 - protein, 90 aa, synthetic construct
MLPAPKNLVISRVTEDSARLSWTAPDAAFDSFFIYYIESYPAGEAIVLTVPGSERSYDLTGLKPGTEYWVGIDGVKGGRWSTPLSAIFTT

SEQ ID NO: 42 - protein, 90 aa, synthetic construct
MLPAPKNLVVSRVTEDSARLSWTAPDAAFDSFTIEYYESFYGGEAIVLTVPGSERSYDLTGLKPGTEYYVSIYGVKGGWLSRPLSAIFTT

SEQ ID NO: 43 - protein, 90 aa, synthetic construct
MLPAPKNLVVSRVTEDSARLSWTAPDAAFDSFYIEYYESYPGGEAIVLTVPGSERSYDLTGLKPGTEYDVYIYGVKGGYWSRPLSAIFTT

SEQ ID NO: 44 - protein, 90 aa, synthetic construct
MLPAPKNLVVSRVTEDSARLSWTAPDAAFDSFYIEYYESLPDGEAIVLTVPGSERSYDLTGLKPGTEYAVYIYGVKGGYYSRPLSAIFTT

SEQ ID NO: 45 - protein, 90 aa, synthetic construct
MLPAPKNLVVSRVTEDSARLSWTAPDAAFDSFAIYYLESYPEGEAIVLTVPGSERSYDLTGLKPGTEYWVGIDGVKGGTWSSPLSAIFTT

SEQ ID NO: 46 - protein, 90 aa, synthetic construct
MLPAPKNLVVSRVTEDSARLSWTAPDAAFDSFTIEYFEFTGTGEAIVLTVPGSERSYDLTGLKPGTEYYVSIYGVKGGLLSAPLSAIFTT

SEQ ID NO: 47 - protein, 90 aa, synthetic construct
MLPAPKNLVVSRVTEDSARLSWTAPDAAFDSFTIWYAEALGDGEAIVLTVPGSERSYDLTGLKPGTEYFVDIYGVKGGFWSLPLSAIFTT

SEQ ID NO: 48 - protein, 90 aa, synthetic construct
MLPAPKNLVVSRVTEDSARLSWTAPDAAFDSFVIEYFEQFNLGEAIVLTVPGSERSYDLTGLKPGTEYWVGIYGVKGGWLSHPLSAIFTT

SEQ ID NO: 49 - protein, 90 aa, synthetic construct
MLPAPKNLVVSRVTEDSARLSWTAPDAAFDSFGISYLEWWEDGEAIVLTVPGSERSYDLTGLKPGTEYWVSIAGVKGGKRSYPLSAIFTT

SEQ ID NO: 50 - protein, 90 aa, synthetic construct
MLPAPKNLVVSRVTEDSARLSWTAPDAAFDSFVIEYREGAWYGEAIVLTVPGSERSYDLTGLKPGTEYFVDITGVKGGWWSDPLSAIFTT

SEQ ID NO: 51 - protein, 90 aa, synthetic construct
MLPAPKNLVVSRVTEDSARLSWTAPDAAFDSFFIKYIEWWADGEAIVLTVPGSERSYDLTGLKPGTEYLVEIYGVKGGKWSWPLSAIFTT

SEQ ID NO: 52 - protein, 90 aa, synthetic construct
MLPAPKNLVVSRVTEDSARLSWTAPDAAFDSFKISYQEWWEDGEAIVLTVPGSERSYDLTGLKPGTEYWVNISGVKGGVQSYPLSAIFTT

SEQ ID NO: 53 - protein, 90 aa, synthetic construct
MLPAPKNLVVSRVTEDSARLSWTAPDAAFDSFFISYIEWWDLGEAIVLTVPGSERSYDLTGLKPGTEYHVEIFGVKGGTQSYPLSAIFTT

SEQ ID NO: 54 - protein, 90 aa, synthetic construct
MLPAPKNLVVSRVTEDSARLSWTAPDAAFDSFQILYQENAFEGEAIVLTVPGSERSYDLTGLKPGTEYWVYIYGVKGGYPSVPLSAIFTT

SEQ ID NO: 55 - protein, 90 aa, synthetic construct
MLPAPKNLVVSRVTEDSARLSWTAPDAAFDSFIIEYWEFVGYGEAIVLTVPGSERSYDLTGLKPGTEYWVAIYGVKGGDLSKPLSAIFTT

SEQ ID NO: 56 - protein, 90 aa, synthetic construct
MLPAPKNLVVSRVTEDSARLSWTAPDAAFDSFVIEYFEALEGGEAIVLTVPGSERSYDLTGLKPGTEYFVGIYGVKGGPLSKPLSAIFTT

SEQ ID NO: 57 - protein, 90 aa, synthetic construct
MLPAPKNLVVSRVTEDSARLSWTAPDAAFDSFSIKYLEWWQDGEAIVLTVPGSERSYDLTGLKPGTEYYVHIAGVKGGYRSYPLSAIFTT

SEQ ID NO: 58 - protein, 90 aa, synthetic construct
MLPAPKNLVVSRVTEDSARLSWTAPDAAFDSFTIWYAEADGWGEAIVLTVPGSERSYDLTGLKPGTEYFVDIYGVKGGYLSVPLSAIFTT

SEQ ID NO: 59 - protein, 90 aa, synthetic construct
MLPAPKNLVVSRVTEDSARLSWTAPDAAFDSFTIWYAEWEDEGEAIVLTVPGSERSYDLTGLKPGTEYRVEIYGVKGGYPSKPLSAIFTT

SEQ ID NO: 60 - protein, 90 aa, synthetic construct
MLPAPKNLVVSRVTEDSARLSWTAPDAAFDSFTIWYAEAIGHGEAIVLTVPGSERSYDLTGLKPGTEYWVDIWGVKGGQQSKPLSAIFTT

SEQ ID NO: 61 - protein, 94 aa, synthetic construct
MLPAPKNLVVSRVTEDSARLSWRVESRTFDSFLIQYQESEKVGEAIVLTVPGSERSYDLTGLKPGTEYTVSIYGVVWDTRDNPISNPLSAIFTT

SEQ ID NO: 62 - protein, 92 aa, synthetic construct
MLPAPKNLVVSRVTEDSARLSWTAPDAAFDSFPILYLELNHHGEEIVLTVPGSERSYDLTGLKPGTEYWVYIFGVKGGMYSAPLSAIFTTGG

SEQ ID NO: 63 - protein, 4 aa, synthetic construct
GSGS

SEQ ID NO: 64 - protein, 8 aa, synthetic construct
GGGSGGGS

SEQ ID NO: 65 - protein, 25 aa, synthetic construct
  REGION 1..25: note = synthetic sequence
GGGGSGGGGSGGGGSGGGGSGGGGS

SEQ ID NO: 66 - protein, 4 aa, synthetic construct
APAP

SEQ ID NO: 67 - protein, 10 aa, synthetic construct
APAPAPAPAP

SEQ ID NO: 68 - protein, 20 aa, synthetic construct
APAPAPAPAPAPAPAPAPAP

SEQ ID NO: 69 - protein, 40 aa, synthetic construct
  REGION 1..40: note = synthetic sequence
APAPAPAPAPAPAPAPAPAPAPAPAPAPAPAPAPAPAPAP

SEQ ID NO: 70 - protein, 29 aa, synthetic construct
AEAAAKEAAAKEAAAKEAAAKEAAAKAAA

SEQ ID NO: 71-79 - skipped (9 entries)

SEQ ID NO: 80 - protein, 230 aa, Homo sapiens
QNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 81 - protein, 90 aa, synthetic construct
MLPAPKNLVVSRVTEDSARLSWTAPDAAFDSFVIEYREGAWYGEAIVLTVPGSERSYDLTGLKPGTEYAVYIPGVKGGPRSFPLSAIFTT

SEQ ID NO: 82 - protein, 90 aa, synthetic construct
MLPAPKNLVVSRVTEDSARLSWTAPDAAFDSFAIAYVEWWKLGEAIVLTVPGSERSYDLTGLKPGTEYVVPIPGVKGGGHSSPLSAIFTT

SEQ ID NO: 83 - protein, 90 aa, synthetic construct
MLPAPKNLVVSRVTEDSARLSWTAPDAAFDSFYIYYYESSGTGEAIVLTVPGSERSYDLTGLKPGTEYFVDIGGVKGGSYSLPLSAIFTT

SEQ ID NO: 84 - protein, 90 aa, synthetic construct
MLPAPKNLVVSRVTEDSARLSWTAPDAAFDSFPIYYWEVFPAGEAIELDVPGSERSYDLTGLKPGTEYFVRIEGVKGGASSYPLRAEFTT

SEQ ID NO: 85 - protein, 90 aa, synthetic construct
MLPAPKNLVVSRVTEDSARLSWTAPDAAFDSFFIWYWEKSVDGEAIVLTVPGSERSYDLTGLKPGTEYNVGIQGVKGGTPSDPLSAIFTT

SEQ ID NO: 86 - protein, 90 aa, synthetic construct
MLPAPKNLVVSRVTEDSARLSWTAPDAAFDSFIIWYAEWVNDGEAIVLTVPGSERSYDLTGLKPGTEYRVEITGVKGGTWSRPLSAIFTT

SEQ ID NO: 87 - protein, 90 aa, synthetic construct
MLPAPKNLVVSRVTEDSARLSWTAPDAAFDSFAIEYYEPVPAGEAIYLDVPGSERSYDLTGLKPGTEYDVTIYGVKGGYYSHPLFASFTT

SEQ ID NO: 88 - protein, 90 aa, synthetic construct
MLPAPKNLVVSRVTEDSARLSWTAPDAAFDSFWIEYFEWTVGGEAIVLTVPGSERSYDLTGLKPGTEYYVSIYGVKGGWLSPPLSAIFTT

SEQ ID NO: 89 - protein, 90 aa, synthetic construct
MLPAPKNLVVSRVTEDSARLSWTAPDAAFDSFHISYEETPVVGEAIYLRVPGSERSYDLTGLKPGTEYTVAIHGVKGGRESTPLIAPFTT

SEQ ID NO: 90 - protein, 90 aa, synthetic construct
MLPAPKNLVVSRVTEDSARLSWTAPDAAFDSFTIHYWEFDPPGEAIVLTVPGSERSYDLTGLKPGTEYTVYIEGVKGGWWSKPLSAIFTT

SEQ ID NO: 91 - protein, 90 aa, synthetic construct
MLPAPKNLVVSRVTEDSARLSWTAPDAAFDSFVIEYWERTQPGEAIVLTVPGSERSYDLTGLKPGTEYDVWISGVKGGKWSEPLSAIFTT

SEQ ID NO: 92 - protein, 90 aa, synthetic construct
MLPAPKNLVVSRVTEDSARLSWTAPDAAFDSFLIRYWEWYVLGEAIVLTVPGSERSYDLTGLKPGTEYYVEISGVKGGWQSWPLSAIFTT

SEQ ID NO: 93 - protein, 90 aa, synthetic construct
MLPAPKNLVVSRVTEDSARLSWTAPDAAFDSFYIGYLEPGDNGEAIVLTVPGSERSYDLTGLKPGTEYNVSIGGVKGGLGSYPLSAIFTT

SEQ ID NO: 94 - protein, 90 aa, synthetic construct
MLPAPKNLVVSRITEDSARLSWTAPDAAFDSFGIYYYEWWSTGEAIVLTVPGSERSYDLTGPKPGTEYYVKISGVKGGYRSYPLSAIFTT

SEQ ID NO: 95 - protein, 90 aa, synthetic construct
MLPAPKNLVVSRVTEDSARLSWTAPDAAFDSFRISYYEWYDLGEAIVLTVPGSERSYDLTGLKPGTEYWVDIAGVKGGYYSYPLSAIFTT

SEQ ID NO: 96 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLSAIFTT

SEQ ID NO: 97 - protein, 90 aa, synthetic construct
MLPAPKNLVVSRVTEDSARLSWTAPDAAFDSFFISYFEGWASGEAIHLYVPGSERSYDLTGLKPGTEYSVHIQGVKGGQPSTPLSAIFTT

SEQ ID NO: 98 - protein, 90 aa, synthetic construct
MLPAPKNLVVSRITEDSARLSWTAPDAAFDSFDIPYGEFDTIGEAIVLTVPGSERSYDLTGLKPGTEYDVYIEGVKGGHLSWPLSAIFTT

SEQ ID NO: 99 - protein, 90 aa, synthetic construct
MLPAPKNLVVSRVTEDSARLSWTAPDAAFDSFGIQYNEFVFRGEAIVLTVPGSERSYDLTGLKPGTEYFVPISGVKGGDDSRPLSAIFTT

SEQ ID NO: 100 - protein, 90 aa, synthetic construct
MLPAPKNLVVSRVTEDSARLSWTAPDAAFDSFWIEYWEVVGFGEAIVLTVPGSERSYDLTGLKPGTEYWVGIYGVKGGNPSVPLSAIFTT

SEQ ID NO: 101 - protein, 90 aa, synthetic construct
MLPAPKNLVVSRVTEDSARLSWTAPDAAFDSFAIDYDEPINSGEAIVLTVPGSERSYDLTGPKPGTEYEVEIYGVKGGYLSRPLSAIFTT

SEQ ID NO: 102 - protein, 90 aa, synthetic construct
MLPAPKNLVVSRVTEDSARLSWTAPDAAFDSFSIDYDEPQPVGEAIVLTVPGSERSYDLTGLKPGTEYRVDIWGVKGGPTSGPLRATFTT

SEQ ID NO: 103 - protein, 90 aa, synthetic construct
MLLAPKNLVVSRVTEDSARLSWTAPDAAFDSFAIEYFEYTGEGEAIVLTVPGSERSYDLTGLKPGTEYYVGIYGVKGGYLSRPLSAIFTT

SEQ ID NO: 104 - protein, 90 aa, synthetic construct
MLPAPKNLVVSHVTEDSARLSWTAPDAAFDSFDIEYYELVGSGEAIVLTVPGSERSYDLTGLKPGTEYYVAIYGVKGGYLSRPLSAIFTT

SEQ ID NO: 105 - protein, 90 aa, synthetic construct
MLPAPKNLVVSRVTEDSARLSWTAPDAAFDSFGIAYYERSGAGEAIVLTVPGSERSYDLTGLKPGTEYMVYINGVKGGFVSSPLSAIFTT

SEQ ID NO: 106 - protein, 90 aa, synthetic construct
MLPAPKNLVVSRVTEDSARLSWTAPDAAFDSFTIAYEEHGLVGEAIYLRVPGSERSYDLTGLKPGTEYHVGIMGVKGGVFSSPLSAIFTT

SEQ ID NO: 107 - protein, 90 aa, synthetic construct
MLPAPKNLVVSRVTEDSARLSWTAPDAAFDSFFIQYTESHWVGEAIVLTVPGSERSYDLTGLKPGTEYAVPIEGVKGGDSSTPLSAIFTT

SEQ ID NO: 108 - protein, 90 aa, synthetic construct
MLPAPKNLVVSRVTEDSARLSWTAPDAAFDSFTIIYGEVNPYGEAIVLTVPGSERSYDLTGLKPGTEYDVFIEGVKGGHLSWPLSAIFTT

SEQ ID NO: 109 - protein, 90 aa, synthetic construct
MLPAPKNLVVSRVTEDSARLSWTAPDAAFDSFHIAYEELVTEGEAIYLRVPGSERSYDLTGLKPGTEYLVDIEGVKGGHLSSPLSAIFTT

SEQ ID NO: 110 - protein, 90 aa, synthetic construct
MLPAPKNLVVSRVTEDSARLSWTAPDAAFDSFFIHYHEWWEAGEAIVLTVPGSERSYDLTGLKPGTEYLVDIPGVKGGDLSVPLSAIFTT

SEQ ID NO: 111 - protein, 90 aa, synthetic construct
MLPAPKNLVVSRVTEDSARLSWTAPDAAFDSFWIYYYESVGTGEAIVLTVPGSERSYDLTGLKPGTEYFVDISGVKVGTYSLPLSAIFTT

SEQ ID NO: 112 - protein, 90 aa, synthetic construct
MLPAPKNLVVSRVTEDSARLSWTAPDAAFDSFFIAYFEFANPGEAIVLTVPGSERSYDLTGLKPGTEYKVVIQGVKGGTPSEPLSAISTT

SEQ ID NO: 113 - protein, 90 aa, synthetic construct
MLPAPKNLVVSRVTEDSARLSWTAPDAAFDSFDIHYKEHSWWGEAIVLTVPGSERSYDLTGLKPGTEYIVPIPGVKGGGISRPLSAIFTT

SEQ ID NO: 114 - protein, 90 aa, synthetic construct
MLPAPKNLVVSRVTEDSARLSWTAPDAAFDSFAIEYWEAVGSGEAIVLTVPGSERSYDLTGLKPGTEYHVYIYGVKGGYLSLPLSAIFTT

SEQ ID NO: 115 - protein, 91 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLSAIFTTT

SEQ ID NO: 116 - protein, 90 aa, synthetic construct
MLPAPKNLVVSRVTEDSARLSWTAPDAAFDSFSIAYSEVRYDGEAIVLTVPGSERSYDLTGLKPGTEYVVPIGGVKGGGSSSPLSAIFTT

SEQ ID NO: 117 - protein, 90 aa, synthetic construct
MLPAPKNLVVSRVTEDSARLSWTAPDAAFDSFTIPYGEAFNPGEAIVLTVPGSERSYDLTGLKPGTEYDVFIEGVKGGTLSWPLSAIFTT

SEQ ID NO: 118 - protein, 90 aa, synthetic construct
MLPAPKNLVVSRVTEDSARLSWTAPDAAFDSFRILYGEVDPWGEAIVLTVPGSERSYDLTGLKPGTEYDVWIEGVKGGKLSWPLSAIFTT

SEQ ID NO: 119 - protein, 90 aa, synthetic construct
MLPAPKNLVVSRVTEDSARLSWTAPDAAFDSFHIEYEETPQKGEAIFLRVPGSERSYDLTGLKPGTEYVVNIRGVKGGDLSSPLGALFTT

SEQ ID NO: 120 - protein, 90 aa, synthetic construct
MLPAPKNLVVSRVTEDSARLSWTAPDAAFDSFRIEYIEWWVGGEAIVLTVPGSERSYDLTGLKPGTEYWVDIKGVKGGKRSYPLSAIFTT

SEQ ID NO: 121 - protein, 90 aa, synthetic construct
MLPAPKNLVVSRVTEDSARLSWTAPDAAFDSFSIDYPEFPVRGEAIVLTVPGSERSYDLTGPKPGTEYNVTIQGVKGGFPSMPLSAIFTT

SEQ ID NO: 122 - protein, 90 aa, synthetic construct
MLPAPKNLVVSRVTEDSARLSWTAPDAAFDSFQIPYWEQSLGGEAIVLTVPGSERSYDLTGLKPGTEYEVWIEGVKGGDLSFPLSAISTT

SEQ ID NO: 123 - protein, 90 aa, synthetic construct
MLPAPKNLVVSRVTEDSARLSWTAPDAAFDSFVIPYEEYLYTGEAIVLTVPGSERSYDLTGLKPGTEYDVWIEGVKGGLTSWPLSAIFTT

SEQ ID NO: 124 - protein, 90 aa, synthetic construct
MLPAPKNLVVSRVTEDSARLSWTAPDAAFDSFSIDYPEFPVRGEAIVLTVPGSERSYDLTGLKPGTEYAVTIWGVKGGFTSQPLSAIFTT

SEQ ID NO: 125 - protein, 90 aa, synthetic construct
MLPAPKNLVVSRVTEDSARLSWTAPDAAFDSFVIEYFEFVGEGEAIVLTVPGSERSYDLTGLKPGTEYDVGIYGVKGGSLSSPLSAIFTT

SEQ ID NO: 126 - protein, 90 aa, synthetic construct
MLPAPKNLVVSRVTEDSARLSWTAPDAAFDSFSIDYLELGESGEAIVLTVPGSERSYDLTGLKPGTEYWVYIFGVKGGYPSAPLSAIFTT

SEQ ID NO: 127 - protein, 90 aa, synthetic construct
MLPAPKNLVVSRVTEDSARLSWTAPDAAFDSFPIPYGESPPSGEAIVLTVPGSERSYDLTGLKPGTEYVVIIRGVKGGGRSGPLSAISTT

SEQ ID NO: 128 - protein, 90 aa, synthetic construct
MLPAPKNLVVSRVTEDSARLSWTAPDAAFDSFIINYIEIVQYGEAIVLTVPGSERSYDLTGLKPGTEYPESIWGVKGGGASSPLSAIFTT

SEQ ID NO: 129 - protein, 90 aa, synthetic construct
MLPAPKNLVVSRVTEDSARLSWTAPDAAFDSFDIEYYEAVGAGEAIVLTVPGSERSYDLTGLKPGTEYTVGIYGVKGGWLSKPLSVIFTT

SEQ ID NO: 130 - protein, 90 aa, synthetic construct
MLPAPKNLVVSRVTEDSARLSWTAPDAAFDSFHIPYVEAEVPGEAIQLHVPGSERSYDLTGLKPGTEYYVEIWGVKGGFYSPPLIAEFTT

SEQ ID NO: 131 - protein, 90 aa, synthetic construct
MLPAPKNLVVSRVTEDSARLSWTAPDAAFDSFSIDYYEGKGYGEAIVLTVPGSERSYDLTGLKPGTEYQVLISGVKGGKYSLPLSAIFTT

SEQ ID NO: 132 - protein, 90 aa, synthetic construct
MLPAPKNLVVSRVTEDSARLSWTAPDAAFDSFTIVYAEVTYDGEAIVLTVPGSERSYDLTGLKPGTEYDVFIEGVKGGELSWPLSAIFTT

SEQ ID NO: 133 - protein, 90 aa, synthetic construct
MLPAPKNLVVSRVTEDSARLSWTAPDAAFDSFPIVYGEAWVTGEAIVLTVPGSERSYDLTGLKPGTEYDVWIEGVKGGELSWPLSAIFTT

SEQ ID NO: 134 - protein, 90 aa, synthetic construct
MLPAPKNLVVSRVTEDSARLSWTAPDAAFDSFWIDYYERKYVGEAIVLTVPGSERSYDLTGLKPGTEYEVTIYGVKGGWYSDPLSAIFTT

SEQ ID NO: 135 - protein, 90 aa, synthetic construct
MLPAPKNLVVSRVTEDSARLSWTAPDAAFDSFPISYYEMSGLGEAIVLTVPGSERSYDLTGLKPGTEYMVYIFGVKGGLNSLPLSAIFTT

SEQ ID NO: 136 - protein, 90 aa, synthetic construct
MLPAPKNLVVSRVTEDSARLSWTAPDAAFDSFFIYYIESYPAGEAIVLTVPGSERSYDLTGLKPGTEYWMGIDGVKGGRWSTPLSAIFTT

SEQ ID NO: 137 - protein, 90 aa, synthetic construct
MLPAPKNLVVSRVTEDSARLSWTAPDAAFDSFEIEYDEPSVAGEAIVLTVPGSERSYDLTGLKPGTEYRVFIWGVKGGNQSWPLSAIFTT

SEQ ID NO: 138 - protein, 90 aa, synthetic construct
MLPAPKNLVVSRVTEDSARLSWTAPDAAFDSFFIKYIEWWADGEAIVLTVPGSERSYDLTGLKPGTEYLVEIYGVKGGRQSYPLSAIFTT

SEQ ID NO: 139 - protein, 90 aa, synthetic construct
MLPAPKNLVVSRVTEDSARLSWTAPDAAFDSFDISYWESGKYGEAIVLTVPGSESSYDLTGLKPGTEYLVDIFGVKGGYPSEPLSAIFTT

SEQ ID NO: 140 - protein, 90 aa, synthetic construct
MLPAPKNLVVSRVTEDSARLSWTAPDAAFDSFWISYEESDTEGEAIYLRVPGSERSYDLTGLKPGTEYNVTIQGVKGGFPSMPLSAIFTT

SEQ ID NO: 141 - protein, 90 aa, synthetic construct
MLPAPKNLVVSRVTEDSARLSWTAPDAAFDSFVIEYFEQFNLGEAIVLTVPGSERSYDLTGLKPGTEYLVGIYGVKGGWLSHPLSAIFTT

SEQ ID NO: 142 - protein, 90 aa, synthetic construct
MLPAPKNLVVSRVTKDSARLSWTAPDAAFDSFHIAYEEATTYGEAIFLRVPGSERSYDLTGLKPGTEYEVKIHGVKGGADSKPLVAPFTT

SEQ ID NO: 143 - protein, 90 aa, synthetic construct
MLPAPKNLVVSRVTEDSARLSWTAPDAAFDSFHIAYEEADSEGEAIYLRVPGSERSYDLTGLKPGTEYSVNIQGVKGGIVSFPLHAEFTT

SEQ ID NO: 144 - protein, 90 aa, synthetic construct
MLPAPKNLVVSRVTEDSARLSWTAPDAAFDSFIIPYAEVRPDGEAIVLTVPGSERSYDLTGLKPGTEYSVLIHGVKGGKLSLPLSAIFTT

SEQ ID NO: 145 - protein, 90 aa, synthetic construct
MLPAPKNLVVSRVTEDSARLSWTAPDAAFDSFFIPYAEPSPTGEAIVLTVPGSERSYDLTGLKPGTEYDVWIEGVKGGTLSWPLSAIFTT

SEQ ID NO: 146 - protein, 90 aa, synthetic construct
MLPAPKNLVVSRVTEDSARLSWTAPDAAFDSFYIAYAEPRPDGEAIVLTVPGSERSYDLTGLKPGTEYSVLIHGVKGGLLSSPLSAIFTT

SEQ ID NO: 147 - protein, 90 aa, synthetic construct
MLPAPKNLVVSRVTEDSARLSWTAPDAAFDSFFIGYLEPQPPGEAIVLTVPGSERSYDLTGLKPGTEYSVLIHGVKGGRNSDPLSAISTT

SEQ ID NO: 148 - protein, 90 aa, synthetic construct
MLPAPKNLVVSRVTEDSARLSWTAPDAAFDSFHIEYEEQYSTGEAIYLRVPGSERSYDLTGLKPGTEYHVDIEGVKGGRRSFPLNAFFTT

SEQ ID NO: 149 - protein, 90 aa, synthetic construct
MLPAPKNLVVSRVTEDSARLSWTAPDAAFDSFIIPYAEVRPDGEAIVLTVPGSERSYDLTGLKPGTEYSVLIHGVKGGKLSEPLSAIFTT

SEQ ID NO: 150 - protein, 90 aa, synthetic construct
MLPAPKNLVVSRVTEDSARLSWTAPDAAFDSFPIAYAEPSPTGEAIVLTVPGSERSYDLTGLKPGTEYSVLIHGVKGGHLSDPLSAIFTT

SEQ ID NO: 151 - protein, 90 aa, synthetic construct
MLPAPKNLVVSRVTEDSARLSWTAPDAAFDSFFIPYAEPSPTGEAIVLTVPGSERSYDLTGLKPGTEYGVVILGVKGGYGSDPLSAIFTT

SEQ ID NO: 152 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSSPLSAIFTT

SEQ ID NO: 153 - protein, 90 aa, synthetic construct
MLPAPKNLVVSRVTEDSARLSWTAPDAALDSFRIAYTEYFVGGEAIVLTVPGSERSYDLTGLKPGTEYGVGIYGVKGGAGSSPLSAIFTT

SEQ ID NO: 154 - protein, 90 aa, synthetic construct
MLPAPKNLVVSRVTEDSARLSWTAPDAAFDSFFIPYAEPRPDGEAIVLTVPGSERSYDLTGLKPGTEYSVLIHGVKGGLLSSPLSAIFTT

SEQ ID NO: 155 - protein, 90 aa, synthetic construct
MLPAPKNLVVSRVTEDSARLSWTAPDAAFDSFPITYRERSQYGEAIVLTVPGSERSYDLTGLKPGTEYVVPIEGVKGGRGSKPLSAIFTT

SEQ ID NO: 156 - protein, 90 aa, synthetic construct
MLPAPKNLVVSRVTEDSARLSWTAPDAAFDSFAIEYFENLGIGEAIVLTVPGSERSYDLTGLKPGTEYVVNIYGVKGGWLSSPLSAIFTT

SEQ ID NO: 157 - protein, 90 aa, synthetic construct
MLPAPKNLVVSRVTEDSARLSWTAPDAAFDSFAIEYYEYVGNGEAIVLTVPGSERSYDLTGLKPGTEYQVGIYGVKGGYYSRPLSAIFTT

SEQ ID NO: 158 - protein, 90 aa, synthetic construct
MLPAPKNLVVSRVTEDSARLSWTAPDAAFDSFIIDYLELDDYGEAIVLTVPGSERSYDLTGLKPGTEYPVYIYGVKGGLPSTPLSAIFTT

SEQ ID NO: 159 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGRNSDPLSAIFTT

SEQ ID NO: 160 - protein, 90 aa, synthetic construct
MLPAPKNLVVSRVTEDSARLSWTAPDAAFDSFNIAYGEWRQHGEAIVLTVPGSERSYDLTGLKPGTEYDVFIDGVKGGNLSWPLSAIFTT

SEQ ID NO: 161 - protein, 90 aa, synthetic construct
MLPAPKNLVVSRVTEDSARLSWTAPDAAFDSFTIRYWEELPTGEAIVLTVPGSERSYDLTGLKPGTEYTVEIFGVKGGYLSRPLSAISTT

SEQ ID NO: 162 - protein, 90 aa, synthetic construct
MLPAPKNLVVSRVTEDSARLSWTAPDAAFDSFHIAYEEATTYGEAIFLRVPGSERSYDLTGLKPGTEYDVWIEGVKGGTISGPLSAIFTT

SEQ ID NO: 163 - protein, 90 aa, synthetic construct
MLPAPKNLVVSRVTEDSARLSWTAPDAAFDSFPIAYAEPRPDGEAIVLTVPGSERSYDLTGLKPGTEYFVDIFGVKGGTLSRPLSAIFTT

SEQ ID NO: 164 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGHLSDPLSAIFTT

SEQ ID NO: 165 - protein, 90 aa, synthetic construct
MLPARKNLVVSRVTEDSARLSWTAPDAAFDSFFIPYAEPSPTGEAIVLTVPGSERSYDLTGLKPGTEYSVLIHGVKGGHLSDPLSAISTT

SEQ ID NO: 166 - protein, 90 aa, synthetic construct
MLPAPKNLVVSRVTEDSARLSWTAPDAAFDSFTILYNEIQNVGEAIVLTVPGSERSYDLTGLKPGTEYDVWIEGVKGGELSWPLSAIFTT

SEQ ID NO: 167 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGTPSEPLSAIFTT

SEQ ID NO: 168 - protein, 90 aa, synthetic construct
MLPAPKNLVVSRVTEDSARLSWTTPDAAFDSFFIGYLEPYPPGEAIVLTVPGSERSYDLTGLKPGTEYVVSIQGVKGGKPSDPLSAIFTT

SEQ ID NO: 169 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSVPLSAIFTT

SEQ ID NO: 170 - protein, 90 aa, synthetic construct
MLPAPKNLVVSRVTEDSARLSWTAPDAAFDSFFIGYPEYPATGEAIVLTVPGSERSYDLTGLKPGTEYFVDINGVKGGSLSYPLSAIFTT

SEQ ID NO: 171 - protein, 90 aa, synthetic construct
MLPAPKNLVVSRVTEDSARLSWTAPDAAFDSFAIRYLEWWDVGEAIVLTVPGSERSYDLTGLKPGTEYLVEIKGVKGGKFSYPLSAIFTT

SEQ ID NO: 172 - protein, 90 aa, synthetic construct
MLPAPKNLVVSRVTEDSARLSWTAPDAAFDSFPIEYDEWWALGEAITLIVPASERSYDLTGLKPGTEYVVKIHGVKGGQRSYPLIAFFTT

SEQ ID NO: 173 - protein, 88 aa, synthetic construct
MLPAPKNLVVSRVTEDSARLSWTAPDAAFDSFTIHYRELYVQAIVLTVPGSERSYDLTGLKPGTEYLVMIPGVKGGPTSVPLSAIFTT

SEQ ID NO: 174 - protein, 90 aa, synthetic construct
MLPAPKNLVVSRVTEDSARLSWTAPDAAFDSFFIGYLEPQPPGEAIVLTVPGSERSYDLTGLKPGTEYKVVIQGVKGGTPSEPLSAIFTT

SEQ ID NO: 175 - protein, 90 aa, synthetic construct
MLPAPKNLVVSRVTEDSARLSWTAPDAAFDSFFIGYLEPQPPGEAIVLTVPGSERSYDLTGLKPGTEYSVVIQGVKGGFPSDPLSAIFTT

SEQ ID NO: 176 - protein, 90 aa, synthetic construct
MLPAPKNLVVSRVTEDSARLSWTAPDAAFDSFPIAYAEPRPDGEAIVLTVPGSERSYDLTGLKPGTEYSVGIHGVKGGHDSSPLSAIFTT

SEQ ID NO: 177 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGRASGPLSAIFTT

SEQ ID NO: 178 - protein, 90 aa, synthetic construct
MLPAPKNLVVSRVTEDSARLSWTAPDAAFDSFDIAYAEPIPRGEAIVLTVPGSERSYDLTGLKPGTEYSVLIHGVKGGRRSVPLSAIFTT

SEQ ID NO: 179 - protein, 90 aa, synthetic construct
MLPAPKNLVVSRVTEDSARLSWTAPDAAFDSFYIAYAEPRPDGEAIVLTVPGSERSYDLTGLKPGTEYPVPIPGVKGGPGSSPLSAIFTT

SEQ ID NO: 180 - protein, 90 aa, synthetic construct
MLPAPKNLVVSRVTEDSARLSWTAPDAAFDSFEISYYEMRGYGEAIVLTVPGSERSYDLTGLKPGTEYSVLIHGVEGGDYSSPLSAISTT

SEQ ID NO: 181 - protein, 90 aa, synthetic construct
MLPAPKNLVVSHVTEDSARLSWTAPDAAFDSFPIAYAEPRPDGEAIVLTVPGSERSYDLTGLKPGTEYSVLIHGVKGGLLSSPLSAIFTT

SEQ ID NO: 182 - protein, 90 aa, synthetic construct
MLPAPKNLVVSRVTEDSARLSWTAPDAAFDSFFIGYLEPYPPGEAIVLTVPGSERSYDLTGLKPGTEYVVSIQGVKGGTPSQPLSAIFTT

SEQ ID NO: 183 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGRPSNPLVAAFTT

SEQ ID NO: 184 - protein, 90 aa, synthetic construct
MLPAPKNLVVSRITEDSARLSWTAPDAAFDSFGIGYYEHKRFGEAIQLSVPGSERSYDLTGLKPGTEYEVDIEGVKGGVLSWPLFAEFTT

SEQ ID NO: 185 - protein, 90 aa, synthetic construct
MLPAPKNLVVSRVTEDSARLSWTAPDAAFDSFEIDYDELAIYGEAIVLTVPGSERSYDLTGLKPGTEYGVMIIGVKGGLPSDPLSAIFTT

SEQ ID NO: 186 - protein, 87 aa, synthetic construct
MLPAPKNLVVSRVTEDSARLSWTAPDAAFDSFFIGYLESAEAIVLTVPGSERSYDLTGLKPGTEYLVTIQGVKGGIASDPLSAIFTT

SEQ ID NO: 187 - protein, 90 aa, synthetic construct
MLPAPKNLVVSRVTEDSARLSWTAPDAAFDSFVIEYFEFVGYGEAIVLTVPGSERSYDLTGLKPGTEYSVGIYGVKGGKLSPPLSAIFTT

SEQ ID NO: 188 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGKLSLPLSAIFTT

SEQ ID NO: 189 - protein, 86 aa, synthetic construct
MLPAPKNLVVSRVTEDSARLSWTAPDAAFDSFHEWVYFGEAIVLTVPGSERSYDLTGLKPGTEYFVDIWGVKGGTVSKPLSAIFTT

SEQ ID NO: 190 - protein, 90 aa, synthetic construct
MLPAPKNLVVSRVTEDSARLSWTAPDAAFDSFFIGYPEYPATGEAITLFVPGSERSYDLTGLKPGTEYNVVIQGVKGGRPSNPLVVAFTT

SEQ ID NO: 191 - protein, 90 aa, synthetic construct
MLPAPENLVVSRVTEDSARLSWTAPDAAFDSFEITYEENWRRGEAIVLTVPGSERSYDLTGPKPGTEYIVIIQGVKGGAESWPLSAIFTT

SEQ ID NO: 192 - protein, 90 aa, synthetic construct
MLPAPKNLVVSRVTEDSARLSWTALDAAFDSFFIGYLEPQPPGEAIVLTVPGSERSYDLTGLKPGTEYNVTIQGVKGGFPSMPLSAIFTT

SEQ ID NO: 193 - protein, 90 aa, synthetic construct
MLPAPKNLVVSRVTEDSARLSWTAPDAAFDSFTIWYAEAVGNGEAIVLTVPGSERSYDLTGLKPGTEYWVDIWGVKGGEFSSPLSAIFTT

SEQ ID NO: 194 - protein, 90 aa, synthetic construct
MLPAPKNLVVSRVTEDSARLSWTAPDAAFDSFEIDYDELAIYGEAIVLTVPGSERSYDLTGLKPGTEYRVFIYGVKGGWTSWPLSTIFTT

SEQ ID NO: 195 - protein, 90 aa, synthetic construct
MLPAPKNLVVSRVTEDSARLSWTAPDAAFDSFPIEYDEIPFWGEAIVLTVPGSERSYDLTGLKPGTEYRVWIHGVKGGNSSWPLSAIFTT

SEQ ID NO: 196 - protein, 90 aa, synthetic construct
MLPAPKNLVVSRVTEDSARLSWTAPDAAFDSFNIHYVEWWVLGEAIVLTVPGSERSYDLTGLKPGTEYPVYIYGVKGGPKSIPLSAIFTT

SEQ ID NO: 197 - protein, 90 aa, synthetic construct
MLPAPKNLVVSRVTEDSARLSWTAPDAAFDSFKIDYLEINDNGEAIVLTVPGSERSYDLTGLKPGTEYPVYIWGVKGGYPSSPLSAIFTT

SEQ ID NO: 198 - protein, 90 aa, synthetic construct
MLPAPKNLVVSRVTEDSARLSWTAPDAAFDSFLIAYNEDRKFGEAIVLTVPGSERSYDLTGLKPGTEYDVWIEGVKGGSLSFPLSAIFTT

SEQ ID NO: 199 - protein, 90 aa, synthetic construct
MLPAPKNLVVSRVTEDSARLSWTAPDAAFDSFGIRYFEWWDLGEAIVLTVPGSERSYDPTGLKPGTEYNVTIQGVKGGFPSMPLSAIFTT

SEQ ID NO: 200 - protein, 90 aa, synthetic construct
MLPAPKNLVVSRVTEDSARLSWTAPDAAFDSFYIEYYEWMHTGEAIVLTVPGSERSYDLTGLKPGTEYSVYIYGVKGGYPSSPLSAIFTT

SEQ ID NO: 201 - protein, 90 aa, synthetic construct
MLPAPKNLVVSRVTEDSARLSWTAPDAAFDSFNIDYWETWVIGEAIVLTVPGSERSYDLTGLKPGTEYEVIIPGVKGGTISPPLSAIFTT

SEQ ID NO: 202 - protein, 90 aa, synthetic construct
MLPAPKNLVVSRVTEDSARLSWTAPDAAFDSFYIDYLELTYSGEAIVLTVPGSERSYDLTGLKPGTEYYVYIYGVKGGYPSSPLSAIFTT

SEQ ID NO: 203 - protein, 90 aa, synthetic construct
MLPAPKNLVVSRVTEDSARLSWTAPDAALDSFRIEYYESYGHGEAIVLTVPGSERSYDLTGLKPGTEYDVGIYGVKGGYYSRPLSAIFTT

SEQ ID NO: 204 - protein, 90 aa, synthetic construct
MLPAPKNLVVSRVTEDSARLPWTAPDAAFDSFWISYYESVGYGEAIVLTVPGSERSYDLTGLKPGTEYYVDISGVKGGVYSLPLSAIFTT

SEQ ID NO: 205 - protein, 90 aa, synthetic construct
MLPAPKNLVVSRVTEDSARLSWTAPDAAFDSFDIDYDEPAWNGEAIVLTVPGSERSYDLTGLKPGTEYRVFIYGVKGGNTSWPLSAIFTT

SEQ ID NO: 206 - protein, 90 aa, synthetic construct
MLPAPKNLVVSRVTEDSARLSWTAPDAAFDSFDIEYDELWKNGEAIVLTVPGSERSYDLTGLKPGTEYRVFIYGVKGGYGSFPLSAIFTT

SEQ ID NO: 207 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGTPSEPLSAISTT

SEQ ID NO: 208 - protein, 90 aa, synthetic construct
MLPAPKNLVVSRVTEDSARLSWTAPDAAFDSFGIVYREPYVGGEAIVLTVPGSERSYDLTGLKPGTEYGVPIPGVKGGYDSGPLSAIFTT

SEQ ID NO: 209 - protein, 90 aa, synthetic construct
MLPAPKNLVVSRVTEDSARLSWTAPDAAFDSFTIPYIEYVWWGEAIVLTVQGSERSYDLTGLKPGTEYPVTIGGVKGGSRSHPLHAHFTT

SEQ ID NO: 210 - protein, 90 aa, synthetic construct
MLPAPKNLVVSRVTEDSARLSWTAPDAAFDSFSIVYGERFVNGEAIVLTVPGSERSYDLTGLKPGTEYHVYIDGVKGGDLSWPLSAIFTT

SEQ ID NO: 211 - protein, 90 aa, synthetic construct
MLPAPKNLVVSRVTEDSARLSWTAPDAAFDSFWINYYEAQPDGEAIVLTVPGSERSYDLTGLKPGTEYDVEIAGVKGGTASLPLSAIFTT

SEQ ID NO: 212 - protein, 90 aa, synthetic construct
MLPAPKNLVVSRVTEDSARLSWTAPDAAFDSFIIEYWEQIGVGEAIVLTVPGSERSYDLTGLKPGTEYWVGIYGVKGGLLSSPLSAIFTT

SEQ ID NO: 213 - protein, 90 aa, synthetic construct
MLPAPKNLVVSRVTEDSARLSWTAPDAAFDSFWIYYWEIERAGEAIRLDVPGSERSYDLTGLKPGTEYRVDIWGVKGGPTSGPLRATFTT

SEQ ID NO: 214 - protein, 90 aa, synthetic construct
MLPAPKNLVVSRVTEDSARLSWTAPDAAFDSFAIPYGERQELGEAIVLTVPGSERSYDLTGLKPGTEYFVVIQGVKGGQPSYPLSAIFTT

SEQ ID NO: 215 - protein, 90 aa, synthetic construct
MLPAPKNLVVSRVTEDSARLSWTAPDAAFDSFLIPYAETSPTGEAIVLTVPGSERSYDLTGLKPGTEYSVLIHGVKGGYPSSPLSAIFTT

SEQ ID NO: 216 - protein, 90 aa, synthetic construct
MLPAPKNLVVSRVTEDSARLSWTAPDAAFDSFPIAYAEPTPSGEAIVLTVPGSERSYDLTGLKPGTEYSVLIHGVKGGGLSLPLSAIFTT

SEQ ID NO: 217 - protein, 90 aa, synthetic construct
MLPAPKNLVVSRVTEDSARLSWTAPDAAFDSFIIEYWEWYFAGEAIVLTVPGSERSYDLTGLKPGTEYTVWITGVKGGTWSEPLSAIFTT

SEQ ID NO: 218 - protein, 90 aa, synthetic construct
MLPAPKNLVVSRVTEDSARLSWTAPDAAFDSFTILYYEMVGEGEAIVLTVPGSERSYDLTGPKPGTEYWVDIYGVKGGGWSRPLSAIFTT

SEQ ID NO: 219 - protein, 90 aa, synthetic construct
MLPAPKNLVVSRVTEDSARLSWTAPDAAFDSFHIDYLELTYAGEAIVLTVPGSERSYDLTGLKPGTEYYVTIYGVKGGYPSSPLSAIFTT

SEQ ID NO: 220 - protein, 90 aa, synthetic construct
MLPAPKNLVVSRVTEDSARLSWTAPDAAFDSFHIIYEEDGTEGEAIYLRVPGSERSYDLTGLKPGTEYEVDIEGVKGGVLSWPLFAEFTT

SEQ ID NO: 221 - protein, 90 aa, synthetic construct
MLPAPKNLVVSRVTEDSARLSWTAPDAAFDSFHISYQEVVAEGEAIYLRVPGSERSYDLTGLKPGTEYYVLIHGVKGGYESKPLDASFTT

SEQ ID NO: 222 - protein, 90 aa, synthetic construct
MLPAPKNLVVSRVTEDSARLSWTAPDAAFDSFYIEYFEWTGSGEAIVLTVPGSERSYDLTGLKPGTEYNVAIYGVKGGAVSYPLSAIFTT

SEQ ID NO: 223 - protein, 90 aa, synthetic construct
MLPAPKNLVVSRVTEDSARLSWTAPDAAFDSFTIWYAEALGDGEAIVLTVPGSERSYDLTGLKPGTEYFVDIPGVKGGTRSSPLSAISTT

SEQ ID NO: 224 - protein, 88 aa, synthetic construct
MLLAPKNLVVSRVTEDSARLSWTAPDAAFDSFRYLEQGLYGEAIVLTVPGSERSYDLTGLKPGTEYWVEIIGVKGGEYSTPLSAIFTT

SEQ ID NO: 225 - protein, 90 aa, synthetic construct
MLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 226 - protein, 90 aa, synthetic construct
MLPAPKNLVVSRVTEDSARLSWTAPDAAFDSFHIIYEEVLTEGEAIYLRVPGSERSYDLTGLKPGTEYGVTIKGVKGGAYSIPLIATFTT

SEQ ID NO: 227 - protein, 90 aa, synthetic construct
MLPAPKNLVVSRVTEDSARLSWTAPDAAFDSFRIRYLEWWNIGEAIVLTVPGSERSYDLTGLKPGTEYHVDIWGVKGGYSSYPLSAIFTT

SEQ ID NO: 228 - protein, 90 aa, synthetic construct
MLPAPKNLVVSRVTEDSARLSWTAPDAAFDSFEIYYVEWSEAGEAIVLTVPGSERSYDLTGLKPGTEYRVEIRGVKGGSWSSPLSAIFTT

SEQ ID NO: 229 - protein, 90 aa, synthetic construct
MLPAPKNLVVSRVTEDSARLSWTAPDAAFDSFSIHYDEDWRRGEAIVLTVPGSERSYDLTGLKPGTEYLVEIPGVKGGKASYPLSAIFTT

SEQ ID NO: 230 - protein, 90 aa, synthetic construct
MLPAPKNLVVSRVTEDSARLSWTAPDAAFDSFQIRYPKRWISGEAIVLTVPGSERSYDLTGLKPGTEYEVVIRGVKGGEYSWPLSAIFTT

SEQ ID NO: 231 - protein, 90 aa, synthetic construct
MLPAPKNLVVSRVTEDSARLSWTAPDAAFDSFEIPYIETVALGEAIVLTVPGSERSYDLTGLKPGTEYYVEIYGVKGGSYSYPLSAISTT

SEQ ID NO: 232 - protein, 90 aa, synthetic construct
MLPAPKNLVVSRVTEDSARLSWTAPDAAFDSFTIAYDETLNLGEAIVLTVPGSERSYDLTGLKPGTEYIVGIFGVKGGTHSWPLSAIFTT

SEQ ID NO: 233 - protein, 90 aa, synthetic construct
MLPAPKNLVVSRVTEDSARLSWTAPDAAFDSFYIVYAEPIPNGEAIVLTVPGSERSYDLTGLKPGTEYSVLIHGVKGGRNSDPLSAIFTT

SEQ ID NO: 234 - protein, 90 aa, synthetic construct
MLPAPKNLVVSRVTEDSARLSWTAPDAAFDSFYITYWETWDYGEAIVLTVPGSERSYDLTGLKPGTEYKVPITGVKGGGPSVPLSAIFTT

SEQ ID NO: 235 - protein, 90 aa, synthetic construct
MLPAPKNLVVSRVTEDSARLSWTAPDAAFDSFSINYREWWSDGEAIYLPVPGSERSYDLTGLKPGTEYAVYIQGVKGGSRSFPLHAWFTT

SEQ ID NO: 236 - protein, 90 aa, synthetic construct
MLPAPKNLVVSRVTEDSARLSWTAPDAAFDSFWIEYYEELGSGEAIVLTVPGSERSYDLTGLKPGTEYRVYIYGVKGGYPSSPLSAIFTT

SEQ ID NO: 237 - protein, 90 aa, synthetic construct
MLPAPKNLVVSRVTEDSARLSWTAPDAAFDSFTILYGEMGTTGEAIVLTVPGSERSYDLTGLKPGTEYDVFIEGVKGGELSWPLSAIFTT

SEQ ID NO: 238 - protein, 88 aa, synthetic construct
MLPAPKNLVVSRVTEDSARLSWTAPDAAFDSFKIFYQEFGGEAIVLTVPGSERSYDLTGLKPGTEYWVDIYGVKGGYTSSPLSAIFTT

SEQ ID NO: 239 - protein, 90 aa, synthetic construct
MLPAPKNLVVSRVTEDSARLSWTAPDAAFDSFAITYYEGRWRGEAIVLTVPGSERSYDLTGLKPGTEYGVPIRGVKGGTGSLPLSAIFTT

SEQ ID NO: 240 - protein, 90 aa, synthetic construct
MLPAPKNLVVSRVTEDSARLSWTAPDAAFDSFRIKYLEWWLGGEAIVLTVPGSERSYDLTGLKPGTEYWVDIQGVKGGVLSWPLSAIFTT

SEQ ID NO: 241 - protein, 90 aa, synthetic construct
MLPAPKNLVVSRVTEDSARLSWTAPDAAFDSFNIYYYEWFVSGEAIVLTVPGSERSYDLTGLKPGTEYFVDIDGVKGGYRSRPLSAIFTT

SEQ ID NO: 242 - protein, 90 aa, synthetic construct
MLPAPKNLVVSRVTEDSARLSWTAPDAAFDSFPIKYLEWWSWGEAIVLTVPGSERSYDLTGLKPGTEYRVPISGVKGGGMSGPLSAIFTT

SEQ ID NO: 243 - protein, 90 aa, synthetic construct
MLPAPKNLVVSRVTEDSARLSWTAPDAAFDSFAIPYYEWVNHGEAIVLTVPGSERSYDLTGLKPGTEYPVGIDGVKGGGPSWPLSAIFTT

SEQ ID NO: 244 - protein, 89 aa, synthetic construct
MLPAPKNLVVSRVTEDSARLSWTAPDAAFDSFHIDYSEFHLRGEAIVLTVPGSERSYDLTGLKPGTEYLGIFGVKGGEQSGPLSAIFTT

SEQ ID NO: 245 - protein, 90 aa, synthetic construct
MLPAPKNLVVSRITEDSARLSWTAPDAAFDSFGIAYNEGDHYGEAIVLTVPGSERSYDLTGLKPGTEYSVWIEGVKGGNLSYPLSAIFTT

SEQ ID NO: 246 - protein, 90 aa, synthetic construct
MLPAPKNLVVSRVTEDSARLSWTAPDAAFDSFTIAYNEQNHYGEAIVLTVPGSERSYDLTGLKPGTEYGVWIEGVKGGTLSWPLSAIFTT

SEQ ID NO: 247 - protein, 87 aa, synthetic construct
MLPAPKNLVVSRVTEDSARLSWTAPDAAFDSFYIEWTYKGEAIVLTVPGSERSYDLTGLKPGTEYFVGIPGVKGGKSSYPLSAIFTT

SEQ ID NO: 248 - protein, 92 aa, synthetic construct
MGSLPAPKNLVVSRVTEDSARLSWTAPDAAFDSFAIPYAEPSPTGEAIVLTVPGSERSYDLTGLKPGTEYPVWIQGVKGGSPSAPLSAEFTT

SEQ ID NO: 249 - protein, 90 aa, synthetic construct
MLPAPKNLVVSRVTEDSARLSWTAPDAAFDSFSIDYFESVGFGEAIVLTVPGSERSYDLTGLKPGTEYDVQITGVKGGPHSLPLSAIFTT

SEQ ID NO: 250 - protein, 90 aa, synthetic construct
MLPAPKNLVVSRVTEDSARLSWTAPDAAFDSFFIGYLEPYPPGEAIVLTVPGSERSYDLTGLKPGTEYAVEIAGVKGGLLSSPLSAISTT

SEQ ID NO: 251 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLSAIVTT

SEQ ID NO: 252 - protein, 90 aa, synthetic construct
MLPAPKNLVVSRVTEDSARLSWTAPDAAFDSFDIGYTEYGGYGEAIVLTVPGSERSYDLTGLKPGTEYWVLIQGVKGGGSSVPLSAIFTT

SEQ ID NO: 253 - protein, 90 aa, synthetic construct
MLPAPKNLVVSRVTEDSARLSWTAPDAAFDSFTIEYWETIGGGEAIVLTVPGSERSYDLTGLKPGTEYYVGIYGVKGGWWSRPLSAIFTT

SEQ ID NO: 254 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAISTT

SEQ ID NO: 255 - protein, 90 aa, synthetic construct
MLPAPKNLVVSRVTEDSARLSWTAPDAAFDSFDIEYYELIGRGEAIVLTVPGSERSYDLTGLKPGTEYWVGIYGVKGGWLSRPLSAIFTT

SEQ ID NO: 256 - protein, 90 aa, synthetic construct
MLPAPKNLVVSRVTEDSARLSWTAPDAAFDSFHIVYHEPRPSGEAIVLTVPGSERSYDLTGLKPGTEYEVGIVGVKGGDLSVPLSAIFTT

SEQ ID NO: 257 - protein, 90 aa, synthetic construct
MLPAPKNLVVSRVTEDSARLSWTAPDAAFDSFHIVYHEPRPSGEAIVLTVPGSERSYDLTGLKPGTEYEVGIVSVKGGDLSVPLSAIFTT

SEQ ID NO: 258 - protein, 90 aa, synthetic construct
MLPAPKNLVVSRVTEDSARLSWTAPDAAFDSFFIPYAEPSPTGEAIVLTVPGSERSYDLTGLKPGTEYDVWIEGVKGGVLSWPLSAIFTT

SEQ ID NO: 259 - protein, 90 aa, synthetic construct
MLPAPKNLVVSRVTEDSARLSWTAPDAAFDSFTIEYFEFVDAGEAIVLTVPGSERSYDLTGLKPGTEYWVEIWGVKGGSWSKPLSAIFTT

SEQ ID NO: 260 - protein, 89 aa, synthetic construct
MLPAPKNLVVSRVTEDSARLSWTAPDAAFDSFNISYYEYFVHGEAIVLTVPGSERSYDLTGLKPGTEYYVIDGVKGGDPSEPLSAIFTT

SEQ ID NO: 261 - protein, 90 aa, synthetic construct
MLPAPKNLVVSRVTEDSARLSWTAPDAAFDSFTIVYGEWGVPGEAIVLTVPGSERSYDLTGLKPGTEYDVWIEGVKGGDLSWPLSAIVTT

SEQ ID NO: 262 - protein, 90 aa, synthetic construct
MLPAPKNLVVSRVTEDSARLSWTAPDAAFDSFAIEYFEYTGEGEAIVLTVPGSERSYDLTGLKPGTEYYVGIYGVKGGYLSRPLSAIFTT

SEQ ID NO: 263 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLSAISTT

SEQ ID NO: 264 - protein, 90 aa, synthetic construct
MLPAPKNLVVSRVTEDSARLSWTAPDAAFDSFRIKYQEWWVEGEAIVLTVPGSERSYDLTGLKPGTEYVVQIAGVKGGLSSYPLSAIFTT

SEQ ID NO: 265 - protein, 90 aa, synthetic construct
MLPAPKNLVVSRVTEDSARLSWTAPDAAFDSFWIYYIETSHQGEAIVLTVPGSERSYDLTGLKPGTEYFVLIKGVKGGYDSVPLSAIFTT

SEQ ID NO: 266 - protein, 90 aa, synthetic construct
MLPAPKNLVVSRVTEDSARLSWTAPDAAFDSFMIRYQEGTRWGEAIVLTVPGSERSYDLTGLKPGTEYIVMIAGVKGGQISLPLSAIFTT

SEQ ID NO: 267 - protein, 90 aa, synthetic construct
MLPAPKNLVVSRVTEDSARLSWTAPDAAFDSFPIVYSEIHVIGEAIVLTVPGSERSYDLTGLKPGTEYDVWIEGVKGGHLSEPLSAIFTT

SEQ ID NO: 268 - protein, 90 aa, synthetic construct
MLPAPKNLVVSRVTEDSARLSWTAPDAAFDSFTIVYGEAGAFGEAIVLTVPGSERSYDLTGLKPGTEYDVLIEGVKGGNLSWPLSAIFTT

SEQ ID NO: 269 - protein, 90 aa, synthetic construct
MLPAPKNLVVSRVTEDSARLSWTAPDAAFDSFHINYAEVYTKGEAILLTVPGSERSYDLTGLKPGTEYEVYIPGVKGGPFSRPLNAQFTT

SEQ ID NO: 270 - protein, 90 aa, synthetic construct
MLPAPKNLVVSRVTEDSARLSWTAPDAAFDSFLIRYQEWQRWGEAIVLTVPGSERSYDLTGLKPGTEYTVHIAGVKGGMLSLPLSAIFTT

SEQ ID NO: 271 - protein, 90 aa, synthetic construct
MLPAPKNLVVSRVTEDSARLSWTAPDAAFDSFYIPYAETRDDGEAIVLTVPGSERSYDLTGLKPGTEYSVLIHGVKGGDLSSPLSAIFTT

SEQ ID NO: 272 - protein, 90 aa, synthetic construct
MLPAPKNLVVSRVTEDSARLSWTAPDAAFDSFGIPYAESTPTGEAIVLTVPGSERSYDLTGLKPGTEYSVLIHGVKGGHLSDPLSAIFTT

SEQ ID NO: 273 - protein, 85 aa, synthetic construct
MLPAPKNLVVSRVTEDSARLSWTAPDAAFDSFTIFKDGEAIVLTVPGSERSYDLTGLKPGTEYYVYIYGVKGGYPSKPLSAIFTT

SEQ ID NO: 274 - protein, 90 aa, synthetic construct
MLPAPKNLVVSRVTEDSVRLSWTAPDAAFDSFAISYEEWWVHGEAIVLTVPGSERSYDLTGLKPGTEYSVVIPGVKGGLYSWTLSAISTT

SEQ ID NO: 275 - protein, 90 aa, synthetic construct
MLPAPKNLVVSRVTEDSARLSWTAPDAAFDSFLIAYAEVTLHGEAIVLTVPGSERSYDLTGLKPGTEYSVLIHGVKGGRNSDPLSAIFTT

SEQ ID NO: 276 - protein, 90 aa, synthetic construct
MLPAPKNLVVSRVTEDSARLSWTAPDAAFDSFRIDYLELTSLGEAIVLTVPGSERSYDLTGLKPGTEYPVPILGVKGGLSSWPLSAIFTT

SEQ ID NO: 277 - protein, 90 aa, synthetic construct
MLPAPKNLVVSRVTEDSARLSWTAPDAAFDSFWINYYEGIGEGEAIVLTVPGSERSYDLTGLKPGTEYYVDISGVKGGSYSLPLSAIFTT

SEQ ID NO: 278 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGHLSDPLSAIFTT

SEQ ID NO: 279 - protein, 90 aa, synthetic construct
MLPAPKNLVVSRVTEDSARLSWTAPDAAFDSFSIEYYESVGLGEAIVLTVPGSERSYDLTGLKPGTEYDVSIYGVKGGYLSRPLSAIFTT

SEQ ID NO: 280 - protein, 90 aa, synthetic construct
  VARIANT 12: note = Xaa can be any naturally occurring amino acid
MLPAPKNLVVRXVTEDSARLSWTAPDAAFDSFEIEYDEPYRGGEAIVLTVPGSERSYDLTSLKPGTEYPVSIGGVKGGITSDPLSAIFTT

SEQ ID NO: 281 - protein, 90 aa, synthetic construct
MLPAPKNLVVSRVTEDSARLSWTAPDAAFDSFYIDYDEIHDWGEAIVLTVPGSERSYDLTGLKPGTEYAVQIGGVKGGSFSWTLSAIFTT

SEQ ID NO: 282 - protein, 90 aa, synthetic construct
MLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 283 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAIFTT

SEQ ID NO: 284 - protein, 90 aa, synthetic construct; identical to SEQ ID NO: 96

SEQ ID NO: 285 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSSPLSAIFTT

SEQ ID NO: 286 - protein, 90 aa, synthetic construct
MLPAPKNLVVSRVTEDSARLSWTAPDAAFDSFNIYYPEFPVRGEAIVLTVPGSERSYDLTGLKPGTEYVVSIWGVKGGTQSWPLSAIFTT

SEQ ID NO: 287 - protein, 90 aa, synthetic construct
MLPAPKNLVVSRVTEDSARLSWTAPDAAFDSFTIEYHESGPVGEAIVLTVPGSERSYDLTGLKPGTEYMVWIFGVKGGFVSRPLSAIFTT

SEQ ID NO: 288 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSSPLSAISTT

SEQ ID NO: 289 - protein, 90 aa, synthetic construct
MLPAPKNLVVSRVTEDSARLSWTAPDAAFDSFAIPYYEDTNDGEAIVLTVPGSERSYDLTGLKPGTEYWVSIQGVKGGTVSGPLSAIFTT

SEQ ID NO: 290 - protein, 88 aa, synthetic construct
MLPAPKNLVVSRVTEDSARLSWTAPDAAFDSFFYLEQAWGGEAIVLTVPGSERSYDLTGLKPGTEYWVEITGVKGGYASSPLSAIFTT

SEQ ID NO: 291 - protein, 90 aa, synthetic construct
MLPAPKNLVVSRVTEDSARLSWTAPDAAFDSFHIEYEEPETEGEAIYLHVPGSERSYDLTGLKPGTEYKVLIRGVKGGSYSIPLQAPFTT

SEQ ID NO: 292 - protein, 90 aa, synthetic construct
MLPAPKNLVVSRVTEDSARLSWTAPDAAFDSFDIAYWELTPSGEAIELLVPGSERSYDLTGLKPGTEYRVDIIGVKGGFISEPLGATFTT

SEQ ID NO: 293 - protein, 90 aa, synthetic construct
MLPAPKNLVVSRVTEDSARLSWTAPDAAFDSFTIEYWEFTGSGEAIVLTVPGSERSYDLTGLKPGTEYDVSIYGVKGGWLSYPLSAIFTT

SEQ ID NO: 294 - protein, 90 aa, synthetic construct
MLPAPKNLVVSRVTEDSARLSWTAPDAAFDSFSIIYSEWNVTGEAIVLTVPGSERSYDLTGLKPGTEYDVWIEGVKGGGMSKPLSAISTT

SEQ ID NO: 295 - protein, 90 aa, synthetic construct
MLPAPKNLVVSRVTEDSARLSWTAPDAAFDSFFIGYLEPIPSGEAIVLTVPGSERSYDLTGLKPGTEYPVVIQGVKGGHPSQPLSAIFTT

SEQ ID NO: 296 - protein, 90 aa, synthetic construct
MLPAPKNLVVSRVTEDSARLSWTAPDAAFDSFFIGYLEPQPPGEAIILTVPGSERSYDLTGLKPGTEYNVTIQGVKGGFPSMPLSAIFTT

SEQ ID NO: 297 - protein, 90 aa, synthetic construct
MLPAPKNLVVSRVTEDSARLSWTAPDAAFDSFLIPYAETSPSGEAITLFVPGSERSYDLTGLKPGTEYNVVIQGVKGGRPSNPLVAASTT

SEQ ID NO: 298 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAISTT

SEQ ID NO: 299 - protein, 90 aa, synthetic construct
MLPAPKNLVVSRVTEDSARLSWTAPDAAFDSFLIEYWESVGYGEAIVLTVPGSERSYDLTGLKPGTEYWVGIYGVKGGYYSRPLSAIFTT

SEQ ID NO: 300 - protein, 90 aa, synthetic construct
MLPAPKNLVVSRVTEDSARLSWTAPDAAFDSFFIGYLEPQPPGEAIVLTVPGSERSYDLTGLKPGTEYNVTIHGVKGGTPSMPLSAIFTT

SEQ ID NO: 301 - protein, 90 aa, synthetic construct
MLPAPKNLVVSRVTEDSARLSWTAPDAAFDSFEIEYDEPYRGGEAIVLTVPGSERSYDLTSLKPGTEYPVSIGGVKGGITSDPLSAIFTT

SEQ ID NO: 302 - protein, 90 aa, synthetic construct
MLPAPKNLVVSRVTEDSARLSWTAPDAAFDSFDIYYPEYYDRGEAIVLTVPGSERSYDLTGLKPGTEYTVYIDGVKGGGGSGPLSAIFTT

SEQ ID NO: 303 - protein, 90 aa, synthetic construct
MLPAPKNLVVSRVTEDSARLSWTAPDAAFDSFFIAYFEFANPGEAIVLTVPGSERSYDLTGLKPGTEYKVVIQGVKGGTPSEPLSAIFTT

SEQ ID NO: 304 - protein, 90 aa, synthetic construct
MLPAPKNLVVSRVTEDSARLSWTAPDAAFDSFIITYWEHVGDGEAIVLTVPGSERSYDLTGLKPGTEYFVEIYGVKGGYLSKPLSAIFTT

SEQ ID NO: 305 - protein, 90 aa, synthetic construct
MLPAPKNLVVSRVTEDSARLSWTAPDAAFDSFEIDYDEPFVYGEAIVLTVPGSERSYDLTGLKPGTEYRVFIFGVKGGNGSWPLSAIFTT

SEQ ID NO: 306 - protein, 90 aa, synthetic construct
MLPAPKNLVVSRVTEDSARLSWTAPDAAFDSFYIEYFETQGYGEAIVLTVPGSERSYDLTGLKPGTEYYVAIYGVKGGYLSRPLSAIFTT

SEQ ID NO: 307 - protein, 90 aa, synthetic construct
MLPAPKNLVVSRVTEDSARLSWTAPDAAFDSFPITYSEPAHYGEAIVLTVPGSERSYDLTGLKPGTEYHVGIMGVKGGVFSSPLSAIFTT

SEQ ID NO: 308 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAISTT

SEQ ID NO: 309 - protein, 90 aa, synthetic construct
MLPAPKNLVVSRVTEDSARLSWTAPDAAFDSFIIDYLELDQEGEAIVLTVPGSERSYDLTGLKPGTEYAVYIFGVKGGYPSTPLSAIFTT